SEQUENCE LISTING (85 sequences)

SEQ ID NO: 1 - protein, 1250 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGTSESATPESGPGTSTEPSEGSAPGTSTEPSEGSAPGTSESATPESGPGTSTEPSEGSAPGTSTEPSEGSAPGTSESATPESGPGTSTEPSEGSAPGTSTEPSEGSAPGTSTEPSEGSAPGSPAGSPTSTEEGTSTEPSEGSAPG

SEQ ID NO: 2 - protein, 191 aa, Homo sapiens [sequence omitted]

SEQ ID NO: 3 - protein, 1071 aa, Artificial Sequence
  Description of Artificial Sequence Synthetic polypeptide
GGSPGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 4 - DNA, 3213 bp, Artificial Sequence
  Description of Artificial Sequence Synthetic polynucleotide
ggtgggtctccaggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtgcaagcgcaagcggcgcgccaagcacgggaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggtacccctggcagcggtaccgcttcttcctctccaggtagctctaccccgtctggtgctactggctctccaggttctagcccgtctgcatctaccggtaccggcccaggtagcgaaccggcaacctccggctctgaaactccaggtacttctgaaagcgctactccggaatccggcccaggtagcgaaccggctacttccggctctgaaaccccaggttccaccagctctactgcagaatctccgggcccaggttctactagctctactgcagaatctccgggtccaggtacttctcctagcggcgaatcttctaccgctccaggtagcgaaccggcaacctctggctctgaaactccaggtagcgaacctgcaacctccggctctgaaaccccaggtacttctactgaaccttctgagggcagcgcaccaggttctaccagctctaccgcagaatctcctggtccaggtacctctactccggaaagcggctctgcatctccaggttctactagcgaatctccttctggcactgcaccaggtacttctaccgaaccgtccgaaggcagcgctccaggtacctctactgaaccttccgagggcagcgctccaggtacctctaccgaaccttctgaaggtagcgcaccaggtagctctactccgtctggtgcaaccggctccccaggttctagcccgtctgcttccactggtactggcccaggtgcttccccgggcaccagctctactggttctccaggtagcgaacctgctacctccggttctgaaaccccaggtacctctgaaagcgcaactccggagtctggtccaggtagccctgcaggttctcctacctccactgaggaaggtagctctactccgtctggtgcaaccggctccccaggttctagcccgtctgcttccactggtactggcccaggtgcttccccgggcaccagctctactggttctccaggtacctctgaaagcgctactccggagtctggcccaggtacctctactgaaccgtctgagggtagcgctccaggtacttctactgaaccgtccgaaggtagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttc

SEQ ID NO: 5 - protein, 768 aa, Artificial Sequence
  Description of Artificial Sequence Synthetic polypeptide
GEGSGEGSEGEGSEGSGEGEGSEGSGEGEGGSEGSEGEGSEGSGEGEGGEGSGEGEGSGEGSEGEGGGEGSEGEGSGEGGEGEGSEGGSEGEGGSEGGEGEGSEGSGEGEGSEGGSEGEGSEGGSEGEGSEGSGEGEGSEGSGEGEGSEGSGEGEGSEGSGEGEGSEGGSEGEGGSEGSEGEGSGEGSEGEGGSEGSEGEGGGEGSEGEGSGEGSEGEGGSEGSEGEGGSEGSEGEGGEGSGEGEGSEGSGEGEGSGEGSEGEGSEGSGEGEGSEGSGEGEGGSEGSEGEGSGEGSEGEGSEGSGEGEGSEGSGEGEGGSEGSEGEGGSEGSEGEGGSEGSEGEGGEGSGEGEGSEGSGEGEGSGEGSEGEGSEGSGEGEGSEGSGEGEGGSEGSEGEGSEGSGEGEGGEGSGEGEGSGEGSEGEGGGEGSEGEGSEGSGEGEGSEGSGEGEGSEGGSEGEGGSEGSEGEGSEGGSEGEGSEGGSEGEGSEGSGEGEGSEGSGEGEGSGEGSEGEGGSEGGEGEGSEGGSEGEGSEGGSEGEGGEGSGEGEGGGEGSEGEGSEGSGEGEGSGEGSEGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 6 - DNA, 2304 bp, Artificial Sequence
  Description of Artificial Sequence Synthetic polynucleotide
ggtgagggttctggcgaaggttccgaaggtgagggctccgaaggatctggcgaaggtgagggttccgaaggttctggcgaaggtgaaggcggttctgagggatccgaaggtgaaggctccgaaggatctggcgaaggtgaaggtggtgaaggttctggcgaaggtgagggatctggcgaaggctctgaaggtgaaggtggtggtgaaggctctgaaggtgaaggatctggtgaaggtggcgaaggtgagggatctgaaggcggctccgaaggtgaaggcggatctgaaggcggcgaaggtgaaggttccgaaggttctggtgaaggtgaaggatctgaaggtggctccgaaggtgaaggatctgaaggcggttccgaaggtgagggctctgaaggttctggcgaaggtgaaggctctgaaggatctggtgaaggtgaaggttccgaaggttctggtgaaggtgaaggttccgaaggttctggcgaaggtgaaggttctgaaggtggctctgaaggtgaaggcggctctgaaggatccgaaggtgaaggttctggtgaaggctctgaaggtgaaggcggctctgagggttccgaaggtgaaggcggaggcgaaggttctgaaggtgagggatctggtgaaggttctgaaggtgaaggcggttctgaaggttccgaaggtgaaggtggctctgagggatccgaaggtgaaggtggcgaaggatctggtgaaggtgaaggttctgaaggttctggcgaaggtgagggttctggcgaaggttccgaaggtgagggctccgaaggatctggcgaaggtgagggttccgaaggttctggcgaaggtgaaggcggttctgagggatccgaaggtgagggttctggcgaaggttccgaaggtgagggctccgaaggatctggcgaaggtgagggttccgaaggttctggcgaaggtgaaggcggttctgagggatccgaaggtgaaggcggttctgaaggttccgaaggtgaaggtggctctgagggatccgaaggtgaaggtggcgaaggatctggtgaaggtgaaggttctgaaggttctggcgaaggtgagggttctggcgaaggttccgaaggtgagggctccgaaggatctggcgaaggtgagggttccgaaggttctggcgaaggtgaaggcggttctgagggatccgaaggtgaaggctccgaaggatctggcgaaggtgaaggtggtgaaggttctggcgaaggtgagggatctggcgaaggctctgaaggtgaaggtggtggtgaaggctctgaaggtgaaggttccgaaggttctggtgaaggtgaaggttccgaaggttctggcgaaggtgaaggttctgaaggtggctctgaaggtgaaggcggctctgaaggatccgaaggtgaaggatctgaaggtggctccgaaggtgaaggatctgaaggcggttccgaaggtgagggctctgaaggttctggcgaaggtgaaggctctgaaggatctggtgaaggtgaaggatctggcgaaggctccgaaggtgaaggcggttctgaaggtggcgaaggtgaaggatctgaaggtggttccgaaggtgagggatctgaaggtggctctgaaggtgaaggtggcgaaggttctggcgaaggtgaaggtggaggcgaaggttctgaaggtgaaggttccgaaggttctggtgaaggtgagggatctggcgaaggttctgaaggttttccgactattccgctgtctcgtctgtttgataacgctatgctgcgtgcgcaccgtctgcaccagctggcgttcgacacttaccaggaatttgaagaagcgtacattccgaaggaacagaagtactctttcctgcaaaacccgcagacctccctgtgcttcagcgaatctattccgactccgtccaatcgtgaagaaactcagcaaaagtccaatctggagctgctgcgcatctctctgctgctgattcagagctggctggagcctgttcagtttctgcgttccgtcttcgccaacagcctggtttatggtgcttccgacagcaacgtatacgatctgctgaaagatctggaagaaggcattcagaccctgatgggtcgtctggaagatggttctccgcgtactggtcagatcttcaaacaaacttactccaaatttgatactaacagccataacgacgatgctctgctgaaaaactatggtctgctgtattgcttccgcaaggatatggacaaagttgaaaccttcctgcgtattgtgcagtgtcgttccgttgagggcagctgtggtttc

SEQ ID NO: 7 - protein, 1104 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 8 - DNA, 3318 bp, Artificial Sequence
  Description of Artificial Sequence Synthetic polynucleotide
atggctgaacctgctggctctccaacctccactgaggaaggtaccccgggtagcggtactgcttcttcctctccaggtagctctaccccttctggtgcaaccggctctccaggtgcttctccgggcaccagctctaccggttctccaggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttctaa

SEQ ID NO: 9 - protein, 1250 aa, Artificial Sequence; identical to SEQ ID NO: 1
  Description of Artificial Sequence Synthetic polypeptide

SEQ ID NO: 10 - DNA, 3753 bp, Artificial Sequence
  Description of Artificial Sequence Synthetic polynucleotide
atggctgaacctgctggctctccaacctccactgaggaaggtaccccgggtagcggtactgcttcttcctctccaggtagctctaccccttctggtgcaaccggctctccaggtgcttctccgggcaccagctctaccggttctccaggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttctaaggtggtagcgaaccggcaacttccggctctgaaaccccaggtacttctgaaagcgctactcctgagtctggcccaggtagcgaacctgctacctctggctctgaaaccccaggtagcccggcaggctctccgacttccaccgaggaaggtacctctactgaaccttctgagggtagcgctccaggtagcgaaccggcaacctctggctctgaaaccccaggtagcgaacctgctacctccggctctgaaactccaggtagcgaaccggctacttccggttctgaaactccaggtacctctaccgaaccttccgaaggcagcgcaccaggtacttctgaaagcgcaacccctgaatccggtccaggtagcgaaccggctacttctggctctgagactccaggtacttctaccgaaccgtccgaaggtagcgcacca

SEQ ID NO: 11 - protein, 1394 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 12 - DNA, 4185 bp, Artificial Sequence
  Description of Artificial Sequence Synthetic polynucleotide
atggctgaacctgctggctctccaacctccactgaggaaggtaccccgggtagcggtactgcttcttcctctccaggtagctctaccccttctggtgcaaccggctctccaggtgcttctccgggcaccagctctaccggttctccaggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttctaaggtggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcacca

SEQ ID NO: 13 - protein, 1067 aa, Artificial Sequence
  Description of Artificial Sequence Synthetic polypeptide
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 14 - DNA, 3204 bp, Artificial Sequence
  Description of Artificial Sequence Synthetic polynucleotide
ggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtgcaagcgcaagcggcgcgccaagcacgggaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggtacccctggcagcggtaccgcttcttcctctccaggtagctctaccccgtctggtgctactggctctccaggttctagcccgtctgcatctaccggtaccggcccaggtagcgaaccggcaacctccggctctgaaactccaggtacttctgaaagcgctactccggaatccggcccaggtagcgaaccggctacttccggctctgaaaccccaggttccaccagctctactgcagaatctccgggcccaggttctactagctctactgcagaatctccgggtccaggtacttctcctagcggcgaatcttctaccgctccaggtagcgaaccggcaacctctggctctgaaactccaggtagcgaacctgcaacctccggctctgaaaccccaggtacttctactgaaccttctgagggcagcgcaccaggttctaccagctctaccgcagaatctcctggtccaggtacctctactccggaaagcggctctgcatctccaggttctactagcgaatctccttctggcactgcaccaggtacttctaccgaaccgtccgaaggcagcgctccaggtacctctactgaaccttccgagggcagcgctccaggtacctctaccgaaccttctgaaggtagcgcaccaggtagctctactccgtctggtgcaaccggctccccaggttctagcccgtctgcttccactggtactggcccaggtgcttccccgggcaccagctctactggttctccaggtagcgaacctgctacctccggttctgaaaccccaggtacctctgaaagcgcaactccggagtctggtccaggtagccctgcaggttctcctacctccactgaggaaggtagctctactccgtctggtgcaaccggctccccaggttctagcccgtctgcttccactggtactggcccaggtgcttccccgggcaccagctctactggttctccaggtacctctgaaagcgctactccggagtctggcccaggtacctctactgaaccgtctgagggtagcgctccaggtacttctactgaaccgtccgaaggtagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttctaa

SEQ ID NO: 15 - protein, 12 aa, Artificial Sequence
  Description of Artificial Sequence Synthetic peptide
GESPGGSSGSES

SEQ ID NO: 16 - protein, 12 aa, Artificial Sequence
  Description of Artificial Sequence Synthetic peptide
GSEGSSGPGESS

SEQ ID NO: 17 - protein, 12 aa, Artificial Sequence
  Description of Artificial Sequence Synthetic peptide
GSSESGSSEGGP

SEQ ID NO: 18 - protein, 12 aa, Artificial Sequence
  Description of Artificial Sequence Synthetic peptide
GSGGEPSESGSS

SEQ ID NO: 19 - protein, 12 aa, Artificial Sequence
  Description of Artificial Sequence Synthetic peptide
GSPAGSPTSTEE

SEQ ID NO: 20 - protein, 12 aa, Artificial Sequence
  Description of Artificial Sequence Synthetic peptide
GSEPATSGSETP

SEQ ID NO: 21 - protein, 12 aa, Artificial Sequence
  Description of Artificial Sequence Synthetic peptide
GTSESATPESGP

SEQ ID NO: 22 - protein, 12 aa, Artificial Sequence
  Description of Artificial Sequence Synthetic peptide
GTSTEPSEGSAP

SEQ ID NO: 23 - protein, 12 aa, Artificial Sequence
  Description of Artificial Sequence Synthetic peptide
GSTSESPSGTAP

SEQ ID NO: 24 - protein, 12 aa, Artificial Sequence
  Description of Artificial Sequence Synthetic peptide
GTSTPESGSASP

SEQ ID NO: 25 - protein, 12 aa, Artificial Sequence
  Description of Artificial Sequence Synthetic peptide
GTSPSGESSTAP

SEQ ID NO: 26 - protein, 12 aa, Artificial Sequence
  Description of Artificial Sequence Synthetic peptide
GSTSSTAESPGP

SEQ ID NO: 27 - protein, 12 aa, Artificial Sequence
  Description of Artificial Sequence Synthetic peptide
GTPGSGTASSSP

SEQ ID NO: 28 - protein, 12 aa, Artificial Sequence
  Description of Artificial Sequence Synthetic peptide
GSSTPSGATGSP

SEQ ID NO: 29 - protein, 12 aa, Artificial Sequence
  Description of Artificial Sequence Synthetic peptide
GSSPSASTGTGP

SEQ ID NO: 30 - protein, 12 aa, Artificial Sequence
  Description of Artificial Sequence Synthetic peptide
GASPGTSSTGSP

SEQ ID NO: 31 - protein, 48 aa, Artificial Sequence
  Description of Artificial Sequence Synthetic polypeptide
MAEPAGSPTSTEEGTPGSGTASSSPGSSTPSGATGSPGASPGTSSTGS

SEQ ID NO: 32 - protein, 48 aa, Artificial Sequence
  Description of Artificial Sequence Synthetic polypeptide
MAEPAGSPTSTEEGASPGTSSTGSPGSSTPSGATGSPGSSTPSGATGS

SEQ ID NO: 33 - protein, 144 aa, Artificial Sequence
  Description of Artificial Sequence Synthetic polypeptide
GSEPATSGSETPGTSESATPESGPGSEPATSGSETPGSPAGSPTSTEEGTSTEPSEGSAPGSEPATSGSETPGSEPATSGSETPGSEPATSGSETPGTSTEPSEGSAPGTSESATPESGPGSEPATSGSETPGTSTEPSEGSAP

SEQ ID NO: 34 - protein, 144 aa, Artificial Sequence
  Description of Artificial Sequence Synthetic polypeptide
GTSTPESGSASPGTSPSGESSTAPGTSPSGESSTAPGSTSSTAESPGPGSTSESPSGTAPGSTSSTAESPGPGTSPSGESSTAPGTSTPESGSASPGSTSSTAESPGPGTSPSGESSTAPGTSPSGESSTAPGTSPSGESSTAP

SEQ ID NO: 35 - protein, 288 aa, Artificial Sequence
  Description of Artificial Sequence Synthetic polypeptide
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 36 - protein, 504 aa, Artificial Sequence
  Description of Artificial Sequence Synthetic polypeptide
GASPGTSSTGSPGSSPSASTGTGPGSSPSASTGTGPGTPGSGTASSSPGSSTPSGATGSPGSXPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSXPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSP

SEQ ID NO: 37 - protein, 540 aa, Artificial Sequence
  Description of Artificial Sequence Synthetic polypeptide
GSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAP

SEQ ID NO: 38 - protein, 576 aa, Artificial Sequence
  Description of Artificial Sequence Synthetic polypeptide
GSSESGSSEGGPGSGGEPSESGSSGSSESGSSEGGPGSSESGSSEGGPGSSESGSSEGGPGSSESGSSEGGPGSSESGSSEGGPGESPGGSSGSESGSEGSSGPGESSGSSESGSSEGGPGSSESGSSEGGPGSSESGSSEGGPGSGGEPSESGSSGESPGGSSGSESGESPGGSSGSESGSGGEPSESGSSGSSESGSSEGGPGSGGEPSESGSSGSGGEPSESGSSGSEGSSGPGESSGESPGGSSGSESGSGGEPSESGSSGSGGEPSESGSSGSGGEPSESGSSGSSESGSSEGGPGESPGGSSGSESGESPGGSSGSESGESPGGSSGSESGESPGGSSGSESGESPGGSSGSESGSSESGSSEGGPGSGGEPSESGSSGSEGSSGPGESSGSSESGSSEGGPGSGGEPSESGSSGSSESGSSEGGPGSGGEPSESGSSGESPGGSSGSESGESPGGSSGSESGSSESGSSEGGPGSGGEPSESGSSGSSESGSSEGGPGSGGEPSESGSSGSGGEPSESGSSGESPGGSSGSESGSEGSSGPGESSGSSESGSSEGGPGSEGSSGPGESS

SEQ ID NO: 39 - protein, 576 aa, Artificial Sequence
  Description of Artificial Sequence Synthetic polypeptide
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 40 - protein, 576 aa, Artificial Sequence
  Description of Artificial Sequence Synthetic polypeptide
GSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAPGSTSSTAESPGPGTSTPESGSASPGTSTPESGSASP

SEQ ID NO: 41 - protein, 625 aa, Artificial Sequence
  Description of Artificial Sequence Synthetic polypeptide
MAEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 42 - protein, 836 aa, Artificial Sequence
  Description of Artificial Sequence Synthetic polypeptide
GSSESGSSEGGPGSSESGSSEGGPGESPGGSSGSESGSGGEPSESGSSGESPGGSSGSESGESPGGSSGSESGSSESGSSEGGPGSSESGSSEGGPGSSESGSSEGGPGESPGGSSGSESGESPGGSSGSESGESPGGSSGSESGSSESGSSEGGPGSSESGSSEGGPGSSESGSSEGGPGSSESGSSEGGPGSSESGSSEGGPGSSESGSSEGGPGSGGEPSESGSSGESPGGSSGSESGESPGGSSGSESGSGGEPSESGSSGSEGSSGPGESSGSSESGSSEGGPGSGGEPSESGSSGSEGSSGPGESSGSSESGSSEGGPGSGGEPSESGSSGESPGGSSGSESGSGGEPSESGSSGSGGEPSESGSSGSSESGSSEGGPGSGGEPSESGSSGSGGEPSESGSSGSEGSSGPGESSGESPGGSSGSESGSEGSSGPGESSGSEGSSGPGESSGSGGEPSESGSSGSSESGSSEGGPGSSESGSSEGGPGESPGGSSGSESGSGGEPSESGSSGSEGSSGPGESSGESPGGSSGSESGSEGSSGPGSSESGSSEGGPGSGGEPSESGSSGSEGSSGPGESSGSEGSSGPGESSGSEGSSGPGESSGSGGEPSESGSSGSGGEPSESGSSGESPGGSSGSESGESPGGSSGSESGSGGEPSESGSSGSEGSSGPGESSGESPGGSSGSESGSSESGSSEGGPGSSESGSSEGGPGSSESGSSEGGPGSGGEPSESGSSGSSESGSSEGGPGESPGGSSGSESGSGGEPSESGSSGSSESGSSEGGPGESPGGSSGSESGSGGEPSESGSSGESPGGSSGSESGSGGEPSESGSS

SEQ ID NO: 43 - protein, 864 aa, Artificial Sequence
  Description of Artificial Sequence Synthetic polypeptide
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 44 - protein, 875 aa, Artificial Sequence
  Description of Artificial Sequence Synthetic polypeptide
GSTSESPSGTAPGTSPSGESSTAPGSTSESPSGTAPGSTSESPSGTAPGTSTPESGSASPGTSTPESGSASPGSTSESPSGTAPGSTSESPSGTAPGTSPSGESSTAPGSTSESPSGTAPGTSPSGESSTAPGTSPSGESSTAPGSTSSTAESPGPGTSPSGESSTAPGTSPSGESSTAPGSTSSTAESPGPGTSTPESGSASPGTSTPESGSASPGSTSESPSGTAPGSTSESPSGTAPGTSTPESGSASPGSTSSTAESPGPGTSTPESGSASPGSTSESPSGTAPGTSPSGESSTAPGSTSSTAESPGPGTSPSGESSTAPGTSTPESGSASPGSTSSTAESPGPGSTSSTAESPGPGSTSSTAESPGPGSTSSTAESPGPGTSPSGESSTAPGSTSESPSGTAPGSTSESPSGTAPGTSTPESGPXXXGASASGAPSTXXXXSESPSGTAPGSTSESPSGTAPGSTSESPSGTAPGSTSESPSGTAPGSTSESPSGTAPGSTSESPSGTAPGTSTPESGSASPGTSPSGESSTAPGTSPSGESSTAPGSTSSTAESPGPGTSPSGESSTAPGTSTPESGSASPGSTSESPSGTAPGSTSESPSGTAPGTSPSGESSTAPGSTSESPSGTAPGTSTPESGSASPGTSTPESGSASPGSTSESPSGTAPGTSTPESGSASPGSTSSTAESPGPGSTSESPSGTAPGSTSESPSGTAPGTSPSGESSTAPGSTSSTAESPGPGTSPSGESSTAPGTSTPESGSASPGTSPSGESSTAPGTSPSGESSTAPGTSPSGESSTAPGSTSSTAESPGPGSTSSTAESPGPGTSPSGESSTAPGSSPSASTGTGPGSSTPSGATGSPGSSTPSGATGSP

SEQ ID NO: 45 - protein, 864 aa, Artificial Sequence
  Description of Artificial Sequence Synthetic polypeptide
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 46 - protein, 875 aa, Artificial Sequence
  Description of Artificial Sequence Synthetic polypeptide
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 47 - protein, 913 aa, Artificial Sequence
  Description of Artificial Sequence Synthetic polypeptide
MAEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 48 - protein, 924 aa, Artificial Sequence
  Description of Artificial Sequence Synthetic polypeptide
MAEPAGSPTSTEEGASPGTSSTGSPGSSTPSGATGSPGSSTPSGATGSPGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 49 - protein, 1318 aa, Artificial Sequence
  Description of Artificial Sequence Synthetic polypeptide
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGPEPTGPAPSGGSEPATSGSETPGTSESATPESGPGSPAGSPTSTEEGTSESATPESGPGSPAGSPTSTEEGSPAGSPTSTEEGTSESATPESGPGSPAGSPTSTEEGSPAGSPTSTEEGSTSSTAESPGPGSTSESPSGTAPGTSPSGESSTAPGSTSESPSGTAPGSTSESPSGTAPGTSPSGESSTAPGTSTEPSEGSAPGTSESATPESGPGTSESATPESGPGSEPATSGSETPGTSESATPESGPGTSESATPESGPGTSTEPSEGSAPGTSESATPESGPGTSTEPSEGSAPGTSPSGESSTAPGTSPSGESSTAPGTSPSGESSTAPGTSTEPSEGSAPGSPAGSPTSTEEGTSTEPSEGSAPGSSPSASTGTGPGSSTPSGATGSPGSSTPSGATGSPGSSTPSGATGSPGSSTPSGATGSPGASPGTSSTGSPGASASGAPSTGGTSPSGESSTAPGSTSSTAESPGPGTSPSGESSTAPGTSESATPESGPGTSTEPSEGSAPGTSTEPSEGSAPGSSPSASTGTGPGSSTPSGATGSPGASPGTSSTGSPGTSTPESGSASPGTSPSGESSTAPGTSPSGESSTAPGTSESATPESGPGSEPATSGSETPGTSTEPSEGSAPGSTSESPSGTAPGSTSESPSGTAPGTSTPESGSASPGSPAGSPTSTEEGTSESATPESGPGTSTEPSEGSAPGSPAGSPTSTEEGTSESATPESGPGSEPATSGSETPGSSTPSGATGSPGASPGTSSTGSPGSSTPSGATGSPGSTSESPSGTAPGTSPSGESSTAPGSTSSTAESPGPGSSTPSGATGSPGASPGTSSTGSPGTPGSGTASSSPGSPAGSPTSTEEGSPAGSPTSTEEGTSTEPSEGSAP

SEQ ID NO: 50 - protein, 864 aa, Artificial Sequence
  Description of Artificial Sequence Synthetic polypeptide
GTSTEPSEPGSAGTSTEPSEPGSAGSEPATSGTEPSGSGASEPTSTEPGSEPATSGTEPSGSEPATSGTEPSGSEPATSGTEPSGSGASEPTSTEPGTSTEPSEPGSAGSEPATSGTEPSGTSTEPSEPGSAGSEPATSGTEPSGSEPATSGTEPSGTSTEPSEPGSAGTSTEPSEPGSAGSEPATSGTEPSGSEPATSGTEPSGTSEPSTSEPGAGSGASEPTSTEPGTSEPSTSEPGAGSEPATSGTEPSGSEPATSGTEPSGTSTEPSEPGSAGTSTEPSEPGSAGSGASEPTSTEPGSEPATSGTEPSGSEPATSGTEPSGSEPATSGTEPSGSEPATSGTEPSGTSTEPSEPGSAGSEPATSGTEPSGSGASEPTSTEPGTSTEPSEPGSAGSEPATSGTEPSGSGASEPTSTEPGTSTEPSEPGSAGSGASEPTSTEPGSEPATSGTEPSGSGASEPTSTEPGSEPATSGTEPSGSGASEPTSTEPGTSTEPSEPGSAGSEPATSGTEPSGSGASEPTSTEPGTSTEPSEPGSAGSEPATSGTEPSGTSTEPSEPGSAGSEPATSGTEPSGTSTEPSEPGSAGTSTEPSEPGSAGTSTEPSEPGSAGTSTEPSEPGSAGTSTEPSEPGSAGTSTEPSEPGSAGTSEPSTSEPGAGSGASEPTSTEPGTSTEPSEPGSAGTSTEPSEPGSAGTSTEPSEPGSAGSEPATSGTEPSGSGASEPTSTEPGSEPATSGTEPSGSEPATSGTEPSGSEPATSGTEPSGSEPATSGTEPSGTSEPSTSEPGAGSEPATSGTEPSGSGASEPTSTEPGTSTEPSEPGSAGSEPATSGTEPSGSGASEPTSTEPGTSTEPSEPGSA

SEQ ID NO: 51 - protein, 864 aa, Artificial Sequence
  Description of Artificial Sequence Synthetic polypeptide
GSETATSGSETAGTSESATSESGAGSTAGSETSTEAGTSESATSESGAGSETATSGSETAGSETATSGSETAGTSTEASEGSASGTSTEASEGSASGTSESATSESGAGSETATSGSETAGTSTEASEGSASGSTAGSETSTEAGTSESATSESGAGTSESATSESGAGSETATSGSETAGTSESATSESGAGTSTEASEGSASGSETATSGSETAGSETATSGSETAGTSTEASEGSASGSTAGSETSTEAGTSESATSESGAGTSTEASEGSASGSETATSGSETAGSTAGSETSTEAGSTAGSETSTEAGSETATSGSETAGTSESATSESGAGTSESATSESGAGSETATSGSETAGTSESATSESGAGTSESATSESGAGSETATSGSETAGSETATSGSETAGTSTEASEGSASGSTAGSETSTEAGSETATSGSETAGTSESATSESGAGSTAGSETSTEAGSTAGSETSTEAGSTAGSETSTEAGTSTEASEGSASGSTAGSETSTEAGSTAGSETSTEAGTSTEASEGSASGSTAGSETSTEAGSETATSGSETAGTSTEASEGSASGTSESATSESGAGSETATSGSETAGTSESATSESGAGTSESATSESGAGSETATSGSETAGTSESATSESGAGSETATSGSETAGTSTEASEGSASGTSTEASEGSASGSTAGSETSTEAGSTAGSETSTEAGSETATSGSETAGTSESATSESGAGTSESATSESGAGSETATSGSETAGSETATSGSETAGSETATSGSETAGTSTEASEGSASGTSESATSESGAGSETATSGSETAGSETATSGSETAGTSESATSESGAGTSESATSESGAGSETATSGSETA

SEQ ID NO: 52 - protein, 912 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 53 - protein, 146 aa, Artificial Sequence
  Description of Artificial Sequence Synthetic polypeptide
GGTSESATPESGPGTSTEPSEGSAPGTSTEPSEGSAPGTSESATPESGPGTSTEPSEGSAPGTSTEPSEGSAPGTSESATPESGPGTSTEPSEGSAPGTSTEPSEGSAPGTSTEPSEGSAPGSPAGSPTSTEEGTSTEPSEGSAPG

SEQ ID NO: 54 - protein, 48 aa, Artificial Sequence; identical to SEQ ID NO: 31
  Description of Artificial Sequence Synthetic polypeptide

SEQ ID NO: 55 - protein, 48 aa, Artificial Sequence; identical to SEQ ID NO: 32
  Description of Artificial Sequence Synthetic polypeptide

SEQ ID NO: 56 - protein, 8 aa, Artificial Sequence
  Description of Artificial Sequence Synthetic peptide
LTPRSLLV

SEQ ID NO: 57 - protein, 8 aa, Artificial Sequence
  Description of Artificial Sequence Synthetic peptide
LTPRSLLV

SEQ ID NO: 58 - protein, 8 aa, Artificial Sequence
  Description of Artificial Sequence Synthetic peptide
KLTRVVGG

SEQ ID NO: 59 - protein, 8 aa, Artificial Sequence
  Description of Artificial Sequence Synthetic peptide
TMTRIVGG

SEQ ID NO: 60 - protein, 8 aa, Artificial Sequence
  Description of Artificial Sequence Synthetic peptide
SPFRSTGG

SEQ ID NO: 61 - protein, 8 aa, Artificial Sequence
  Description of Artificial Sequence Synthetic peptide
LQVRIVGG

SEQ ID NO: 62 - protein, 8 aa, Artificial Sequence
  Description of Artificial Sequence Synthetic peptide
PLGRIVGG

SEQ ID NO: 63 - protein, 8 aa, Artificial Sequence
  Description of Artificial Sequence Synthetic peptide
IEGRTVGG

SEQ ID NO: 64 - protein, 8 aa, Artificial Sequence
  Description of Artificial Sequence Synthetic peptide
LTPRSLLV

SEQ ID NO: 65 - protein, 8 aa, Artificial Sequence
  Description of Artificial Sequence Synthetic peptide
LGPVSGVP

SEQ ID NO: 66 - protein, 8 aa, Artificial Sequence
  Description of Artificial Sequence Synthetic peptide
VAGDSLEE

SEQ ID NO: 67 - protein, 8 aa, Artificial Sequence
  Description of Artificial Sequence Synthetic peptide
GPAGLGGA

SEQ ID NO: 68 - protein, 8 aa, Artificial Sequence
  Description of Artificial Sequence Synthetic peptide
GXXGLXGX

SEQ ID NO: 69 - protein, 8 aa, Artificial Sequence
  Description of Artificial Sequence Synthetic peptide
GPAGLRGA

SEQ ID NO: 70 - protein, 8 aa, Artificial Sequence
  Description of Artificial Sequence Synthetic peptide
GPXGLXXX

SEQ ID NO: 71 - protein, 8 aa, Artificial Sequence
  Description of Artificial Sequence Synthetic peptide
APLGLRLR

SEQ ID NO: 72 - protein, 8 aa, Artificial Sequence
  Description of Artificial Sequence Synthetic peptide
PALPLVAQ

SEQ ID NO: 73 - protein, 7 aa, Artificial Sequence
  Description of Artificial Sequence Synthetic peptide
ENLYFQG

SEQ ID NO: 74 - protein, 8 aa, Artificial Sequence
  Description of Artificial Sequence Synthetic peptide
ENLYFQGS

SEQ ID NO: 75 - protein, 8 aa, Artificial Sequence
  Description of Artificial Sequence Synthetic peptide
DDDKIVGG

SEQ ID NO: 76 - protein, 8 aa, Artificial Sequence
  Description of Artificial Sequence Synthetic peptide
DDDKIVGG

SEQ ID NO: 77 - protein, 8 aa, Artificial Sequence
  Description of Artificial Sequence Synthetic peptide
LEVLFQGP

SEQ ID NO: 78 - protein, 8 aa, Artificial Sequence
  Description of Artificial Sequence Synthetic peptide
LEVLFQGP

SEQ ID NO: 79 - protein, 8 aa, Artificial Sequence
  Description of Artificial Sequence Synthetic peptide
LPKTGSES

SEQ ID NO: 80 - protein, 8 aa, Artificial Sequence
  Description of Artificial Sequence Synthetic peptide
LPXTGXXS

SEQ ID NO: 81 - protein, 47 aa, Artificial Sequence
  Description of Artificial Sequence Synthetic polypeptide
AEPAGSPTSTEEGTPGSGTASSSPGSSTPSGATGSPGASPGTSSTGS

SEQ ID NO: 82 - protein, 47 aa, Artificial Sequence
  Description of Artificial Sequence Synthetic polypeptide
AEPAGSPTSTEEGASPGTSSTGSPGSSTPSGATGSPGSSTPSGATGS

SEQ ID NO: 83 - protein, 912 aa, Artificial Sequence; identical to SEQ ID NO: 52
  Description of Artificial Sequence Synthetic polypeptide

SEQ ID NO: 84 - protein, 913 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 85 - protein, 144 aa, Artificial Sequence
  Description of Artificial Sequence Synthetic polypeptide
TSESATPESGPGTSTEPSEGSAPGTSTEPSEGSAPGTSESATPESGPGTSTEPSEGSAPGTSTEPSEGSAPGTSESATPESGPGTSTEPSEGSAPGTSTEPSEGSAPGTSTEPSEGSAPGSPAGSPTSTEEGTSTEPSEGSAPG

(19) United States
(12) Patent Application Publication
(10) Pub. No.: US 2014/0162949 A1
(43) Pub. Date: Jun. 12, 2014

(54) TREATMENT WITH HUMAN GROWTH HORMONE ANALOGUES

(71) Applicants: Michael Harazin, San Diego, CA (US); Amunix Operating Inc., (US)

(72) Inventors: Jeffrey L. Cleland, San Carlos, CA (US); George M. Bright, San Mateo, CA (US); Eric Humphriss, Menlo Park, CA (US); Volker Schellenberger, Palo Alto, CA (US); Joshua Silverman, Sunnyvale, CA (US); Willem P. Stemmer, Los Gatos, CA (US); Chia-Wei Wang, Milpitas, CA (US); Nathan Geething, San Juan, PR (US); Benjamin Spink, San Carlos, CA (US)

(73) Assignee: Amunix Operating Inc., MountainView, CA (US)

(21) Appl. No.: 13/829,369

(22) Filed: Mar. 14, 2013

Related U.S. Application Data
(60) Provisional application No. 61/689,390, filed on Jun. 5, 2012.
(60) Provisional application No. 61/663,475, filed on Jun. 22, 2012.
(60) Provisional application No. 61/763,753, filed on Feb. 12, 2013.

(51) Int. Cl. C07K 14/61; G01N 33/566; C07K 14/00

(52) U.S. Cl. CPC C07K 14/61; C07K 14/00; G01N 33/566
USPC 514/7.4; 514/11.4; 514/8.6; 530/399; 436/501

(57) ABSTRACT

The present invention concerns an improved therapeutic regimen for GHD therapy. In particular, the invention concerns methods for bolus dose administration of a human growth hormone-XTEN (hGH-XTEN) fusion protein.

### RELATED APPLICATIONS

This application claims the benefit of U.S. Provisional Application Nos. 61/689,390 filed Jun. 5, 2012, 61/663,475 filed Jun. 22, 2012, and 61/763,753 filed Feb. 12, 2013, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Mar. 13, 2013, is named 32808-738.201_SL.txt and is 255,823 bytes in size.

### BACKGROUND OF THE INVENTION

Human growth hormone (hGH) is naturally secreted from the human anterior pituitary as intermittent pulses lasting from minutes to hours typically occurring during sleep. The rate and extent of hGH secretion decreases with aging and is maximal in puberty in normal healthy well nourished children. hGH binds to the hGH receptor initiating signaling processes involving the STAT (signal transducer and activator of transcription), the MAPK (mitogen-activated protein kinase) and the PI3K (phosphoinositide-3 kinase) pathways. Insulin-like growth factor-I (IGF-I) gene expression is activated from hGH receptor signaling resulting in secretion of IGF-I into the circulation. IGF-I forms a complex with insulin-like growth factor binding protein-3 (IGFBP-3) and the acid labile subunit (ALS). Both IGFBP-3 and ALS expression are also regulated by hGH receptor activation.

In children with growth hormone deficiency (GHD) resulting from lack of expression or secretion of hGH and not caused by a defect in the hGH receptor, replacement therapy with daily injections of rhGH is often prescribed to facilitate near normal growth and development. New bone is formed at the epiphyses in response to hGH and IGF-I resulting in linear growth until the growth plates fuse after puberty. Daily rhGH administration does not mimic the normal endogenous pulses of hGH in non-GHD children, but does result in significant increases in growth with a typical first year growth rate on treatment of 11 cm/yr. Clinical studies of continuous infusion of rhGH with a pump demonstrated comparable growth velocity and IGF-I levels to those achieved with daily rhGH injections (Jørgensen et al. J. Clin Endocrinol Metab. 70(6), 1616-23 (1990); Laursen, T. et al. J Clin Endocrinol Metab. 80(8), 2410-8 (1995); Tauber, M. et al. J Clin Endocrinol Metab. 76(5), 1135-9 (1993)). Therefore, continuous, as well as pulsatile, administration of rhGH is efficacious.

In adulthood, hGH secretion is reduced but remains important to maintaining proper hormone balance and has been shown to facilitate decreases in fat mass and cardiovascular risk factors, and increases in lean body mass, bone mineral density, and quality of life outcomes. Adult GHD may occur as the result of traumatic injury to the brain or surgical removal of a tumor at or near the pituitary. Patients presenting with GHD in childhood may also require continued hGH replacement therapy in adulthood. In some adult GHD patients, there can be abnormally low IGF-I levels. Because IGF-I levels vary by age and sex, each adult patient must be characterized by their individual age and sex-adjusted IGF-I standard deviation score (IGF-I SDS).

The objective of hGH daily therapies is usually to titrate the adult GHD patient with rhGH dose until the patient achieves an IGF-I SDS near the middle of the range (e.g. IGF-I SDS of 0 (Cook et al., 2009 Update. Endocrine Pract. 15 (Suppl 2), 1-29 (2009)). A continuous infusion of rhGH was compared to daily rhGH therapy in adult GHD patients (7 per group) for 6 months (Laursen et al., J Clin Endocrinol Metab. 86(3), 1222-8 (2001)). This study indicated that the safety profile and effects on the IGF-I responses were not significantly different between patients treated with continuous infusion of rhGH or daily rhGH therapy.

The safety of daily rhGH therapy has been studied in both GHD children and adults. In some overweight or obese patients, a trend toward increasing fasting and post-prandial insulin levels has been observed. Although generally well tolerated, daily rhGH therapy may cause mild to moderate headache, arthralgia, nausea, vomiting and injection reactions.

Others have reported on various sustained release GH preparations (Cook D M, et al. 2002. J Clin Endocrinol Metab 87(10):4508-4514; Biller B M, et al. 2011. J Clin Endocrinol Metab 96(6):1718-1726; Peter F. et al., 2012. J Clin Endocrinol Metab 97(2):400-407; Fares F. et al, 2010. Endocrinology 151(9):4410-4417; Sondergaard E, et al. 2011. J Clin Endocrinol Metab 96(3):681-688; de Schepper J et al. 2011. European Journal of Endocrinology 165(3):401-409; Bidlingmaier M, et al. 2006. J Clin Endocrinol Metab 91(8):2926-2930). However, there remains a need for alternative GH therapeutics, dosages, and treatment regimens.

VRS-317 is an investigational long-acting rhGH in development for long-term replacement therapy for adults (including adults who experienced a growth hormone-related disorder as children) with GHD. VRS-317 was designed to achieve once-monthly dosing with the anticipation that a reduced frequency of administration (12 versus up to 365 injections per year) would increase treatment adherence and thereby improve overall treatment outcomes. VRS-317 is a novel rhGH fusion protein that was designed to minimize receptor mediated clearance through a reduction in receptor binding achieved without mutations to rhGH by genetically fusing extended recombinant polypeptide (XTEN) amino acid sequences to the N- and C-termini of the native hGH sequence (Cleland et al. 2012, Journal of Pharmaceutical Sciences. 101(8):2744-2754, Epub 2012 Jun. 7). Functionally, the XTEN domains increase the hydrodynamic radius and reduce binding affinity to the GH receptor (GHR), in vitro. Despite reduced binding affinity, durable pharmacodynamics response are seen, in vivo, possibly relating to reduced rates of receptor mediated clearance of VRS-317 (Cleland et al. 2012 supra). VRS-317 was evaluated for safety, tolerability and efficacy in 50 adults with GHD in a 60-day, double-blind, randomized, placebo (PBO)-controlled, single ascending dose escalation studying VRS-317/kg (ClinicalTrials.gov NCT01359488).

### SUMMARY OF THE INVENTION

The present invention concerns an improved therapeutic regimen for growth hormone deficiency (“GHD”) therapy. In particular, the invention concerns methods for bolus dose administration of compositions of fusion proteins comprising human growth hormone fused to one or more extended recombinant polypeptides (XTEN) (the fusion protein hereinafter referred to as “hGH-XTEN”). Accordingly, in one aspect, the present invention concerns a method of treating human GHD with an hGH-XTEN fusion protein.

In one aspect, the present invention provides a method of a method of treating human growth hormone deficiency (GHD) in a human patient with an hGH-XTEN fusion protein as a bolus dose. In one embodiment, the method comprises administering to a human patient with GHD an hGH-XTEN fusion protein comprising (i) an amino acid sequence having at least about 90% sequence identity to SEQ ID NO:1; or (ii) the amino acid sequence of SEQ ID NO:1. In another embodiment, the hGH-XTEN fusion protein is administered as a therapeutically effective bodyweight adjusted bolus dose. In one other embodiment, the bolus dose is (i) between about 0.05 mg/kg and about 3.0 mg/kg; (ii) between about 0.05 mg/kg and about 0.8 mg/kg; or (iii) between about 0.8 mg/kg and about 1.2 mg/kg. In other embodiments, the bolus dose is administered once, every week, every two weeks, every three weeks, or monthly. In one embodiment, the administration of the bolus dose is monthly. In another embodiment, the bolus dose is administered subcutaneously. In another embodiment, the human patient has a serum IGF-I standard deviation score (SDS) between about −2.0 and about 2.0 following administration. In one other embodiment, the IGF-I SDS is selected from the group consisting of greater than about −2.0, greater than about −1.5, greater than about −1.0, greater than about −0.5, greater than about 0, greater than about 0.5, greater than about 1.0, and greater than about 1.5. In an additional embodiment, the human patient exhibits said serum IGF-I SDS following administration of the bolus dose, wherein the administration is once, weekly, every two weeks, every three weeks, or monthly. In one embodiment, the administration of the bolus dose results in a normalization of IGF-I SDS in the human patient for at least about 7 days, at least about 10 days, at least about 14 days, at least about 16 days, or at least about 21 days. In one other embodiment, the bolus dose is selected from the group consisting of about 0.05 mg/kg, about 0.1 mg/kg, about 0.2 mg/kg, about 0.4 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, and about 3.0 mg/kg.

In another aspect, the present invention provides a method of treating human growth hormone deficiency (GHD) in a human patient with an hGH-XTEN fusion protein as a bolus dose that is equivalent to less than an hGH/kg/day dosage. In one embodiment, the method comprises administering to a human patient with GHD an hGH-XTEN fusion protein comprising (i) an amino acid sequence having at least about 90% sequence identity to SEQ ID NO:1; or (ii) the amino acid sequence of SEQ ID NO:1. In another embodiment, the bolus dose is a therapeutically effective bodyweight adjusted bolus dose of the hGH-XTEN fusion protein. In one other embodiment, the bolus dose is equivalent to less than an hGH/kg/day dosage between about 2 μg hGH/kg/day and about 20 μg hGH/kg/day. In an additional embodiment, the bolus dose is administered once, every week, every two weeks, every three weeks, or monthly. In one embodiment, the administration of the bolus dose is monthly. In another embodiment, the bolus dose is administered subcutaneously. In other embodiments, the bolus dose is equivalent to less than an hGH/kg/day dosage selected from the group consisting of about 2 μg hGH/kg/day, about 4 μg hGH/kg/day, about 6 μg hGH/kg/day, about 8 μg hGH/kg/day, about 10 μg hGH/kg/day, about 12 μg hGH/kg/day, about 14 μg hGH/kg/day, about 16 μg hGH/kg/day, about 18 μg hGH/kg/day, about 18.6 μg hGH/kg/day, and about 20 μg hGH/kg/day. In one other embodiment, the hGH/kg/day dosage is over about 30 days. In another embodiment, the human patient has a serum IGF-I standard deviation score (SDS) between about −2.0 and about 2.0 following administration. In one other embodiment, the IGF-I SDS is selected from the group consisting of greater than about −2.0, greater than about −1.5, greater than about −1.0, greater than about −0.5, greater than about 0, greater than about 0.5, greater than about 1.0, and greater than about 1.5. In an additional embodiment, the human patient exhibits said serum IGF-I SDS following administration of the bolus dose, wherein the administration is once, weekly, every two weeks, every three weeks, or monthly.

In one other aspect, the present invention provides a method of a method of treating human growth hormone deficiency (GHD) in a human patient with an hGH-XTEN fusion protein as a bolus dose that is effective to maintain a IGF-I standard deviation score (SDS) in the patient. In one embodiment, the method comprises administering to a human patient with GHD an hGH-XTEN fusion protein comprising (i) an amino acid sequence having at least about 90% sequence identity to SEQ ID NO:1; or (ii) the amino acid sequence of SEQ ID NO:1. In another embodiment, the hGH-XTEN fusion protein is administered as a therapeutically effective bodyweight adjusted bolus dose. In one additional embodiment, the bolus dose is effective to maintain the patient's serum IGF-I standard deviation score (SDS) between about −2.0 and about 2.0 for at least 7 days after administration of the bolus dose. In other embodiments, the bolus dose is (i) between about 0.05 mg/kg and about 0.8 mg/kg; (ii) between about 0.8 mg/kg and about 1.2 mg/kg; or (iii) between about 0.05 mg/kg and about 3.0 mg/kg. In another embodiment, the bolus dose is effective to maintain the patient's serum IGF-I SDS between about −2.0 and about 2.0 for at least 20 days after administration of the bolus dose. In one embodiment, the bolus dose is administered subcutaneously. In another embodiment, the human patient has a clinically significant reduction in at least one parameter selected from serum cholesterol, serum triglycerides, and serum low-density lipoprotein (LDL) after administration of the bolus dose, wherein the administration is selected from the group consisting of once, weekly, every two weeks, every three weeks, and monthly.

In another aspect, the present invention provides a method of a method of treating human growth hormone deficiency (GHD) in a human patient with an hGH-XTEN fusion protein as a bolus dose that is effective to maintain a plasma concentration of said fusion protein in the patient. In one embodiment, the method comprises administering to a human patient with GHD an hGH-XTEN fusion protein comprising (i) an amino acid sequence having at least about 90% sequence identity to SEQ ID NO:1; or (ii) the amino acid sequence of SEQ ID NO:1. In another embodiment, the hGH-XTEN fusion protein is administered as a therapeutically effective bodyweight adjusted bolus dose. In one other embodiment, the bolus dose is effective to maintain a plasma concentration of said fusion protein in the patient at more than about 10 ng/mL for a period of at least 10 days after administration of the bolus dose. In another embodiment, the bolus dose is (i) between about 0.05 mg/kg and about 0.8 mg/kg; (ii) between about 0.8 mg/kg and about 1.2 mg/kg; or (iii) between about 0.05 mg/kg and about 3.0 mg/kg. In one other embodiment, the bolus dose is effective to maintain a plasma concentration of said fusion protein in the patient at more than about 10 ng/mL for a period of at least about 14 days, at least 20 days, at least about 28 days, or at least about 30 days after administration of the bolus dose. In other embodiments, the bolus dose is effective to maintain a plasma concentration of said fusion protein in the patient at more than about 10 ng/mL for a period of at least 20 days or at least about 30 days after administration of the bolus dose. In another embodiment, the bolus dose is effective to maintain a plasma concentration of said fusion protein in the patient at more than about 100 ng/mL for a period of at least 10 days after administration of the bolus dose. In one embodiment, the bolus dose is administered subcutaneously. In another embodiment, the human patient has a clinically significant reduction in at least one parameter selected from serum cholesterol, serum triglycerides, and serum low-density lipoprotein (LDL) after administration of the bolus dose, wherein the administration is selected from the group consisting of once, weekly, every two weeks, every three weeks, and monthly.

In one additional aspect, the present invention provides a method of treating human growth hormone deficiency (GHD) in a human patient with an hGH-XTEN fusion protein as a bolus dose that is effective in increasing the patient's IGF-I SDS in the absence of clinically significant level of side-effects. In one embodiment, the method comprises administering to a human patient with GHD an hGH-XTEN fusion protein comprising (i) an amino acid sequence having at least about 90% sequence identity to SEQ ID NO:1; or (ii) the amino acid sequence of SEQ ID NO:1. In another embodiment, the hGH-XTEN fusion protein is administered as a therapeutically effective bodyweight adjusted bolus dose. In one other embodiment, the bolus dose is effective in increasing the patient's IGF-SDS by at least 0.5 or at least 1.0 above the subject's baseline IGF-I SDS after administration. In one additional embodiment, the increase in IGF-SDS is in the absence of a clinically significant level of side-effects. In one embodiment, the side effect is selected from the group consisting of headache, arthralgia, myalgia, edema, nausea, and muscle fatigue. In one embodiment, the bolus dose is administered subcutaneously.

In another embodiment, the human patient has a clinically significant reduction in at least one parameter selected from serum cholesterol, serum triglycerides, and serum low-density lipoprotein (LDL) after administration of the bolus dose, wherein the administration is selected from the group consisting of once, weekly, every two weeks, every three weeks, and monthly.

In one other aspect, the present invention provides a bolus dose of an hGH-XTEN fusion protein. In one embodiment, the hGH-XTEN fusion protein comprising (i) an amino acid sequence having at least about 90% sequence identity to SEQ ID NO:1; or (ii) the amino acid sequence of SEQ ID NO:1. In another embodiment, the bolus dose is a therapeutically effective bodyweight adjusted bolus dose. In another embodiment, the bolus dose comprises (i) between about 0.05 mg/kg and about 0.8 mg/kg; (ii) between about 0.8 mg/kg and about 1.2 mg/kg; or (iii) between about 0.05 mg/kg and about 3.0 mg/kg, of the hGH-XTEN fusion protein. In one additional embodiment, the bolus dose is for use in treating human GHD in a subject (e.g., a human patient) in need. In one other embodiment, the bolus dose is formulated for subcutaneous administration.

In another aspect, the present invention provides an hGH-XTEN fusion protein for use in a method for the treatment of human GHD in a human patient, wherein the method comprises administering to the patient a bolus dose of the hGH-XTEN fusion protein. In one other aspect, the present invention provides the use of an hGH-XTEN fusion protein in the manufacture of a medicament for the treatment of GHD in a human patient, wherein the hGH-XTEN fusion protein is administered to the patient as a bolus dose. In one embodiment, the hGH-XTEN fusion protein comprising (i) an amino acid sequence having at least about 90% sequence identity to SEQ ID NO:1; or (ii) the amino acid sequence of SEQ ID NO:1. In another embodiment, the bolus dose is a therapeutically effective bodyweight adjusted bolus dose. In one additional embodiment, the bolus dose comprises between about 0.05 mg/kg and about 3.0 mg/kg. In one other embodiment, the bolus dose is administered every week, every two weeks, every three weeks, or monthly. In one embodiment, the bolus dose is administered subcutaneously. In other embodiments, the human patient has a serum IGF-I standard deviation score (SDS) between about −2.0 and about 2.0 following administration of the bolus dose. In another embodiment, the IGF-I SDS is selected from the group consisting of greater than about −2.0, greater than about −1.5, greater than about −1.0, greater than about −0.5, greater than about 0, greater than about 0.5, greater than about 1.0, and greater than about 1.5. In a further embodiment, the administration of the bolus dose is once, weekly, every two weeks, every three weeks, or monthly. In one embodiment, the human patient has a clinically significant reduction in at least one parameter selected from serum cholesterol, serum triglycerides, and serum LDL after administration of the bolus dose, wherein the administration is once, weekly, every two weeks, every three weeks, or monthly.

In one other aspect, the present invention provides a method of increasing the efficacy of human growth hormone (hGH) therapy in a human patient, wherein the hGH therapy comprises the administration of an hGH-XTEN fusion protein. In one embodiment, hGH-XTEN fusion protein comprises (i) an amino acid sequence having at least about 90% sequence identity to SEQ ID NO:1; or (ii) the amino acid sequence of SEQ ID NO:1. In one other embodiment, the method comprises the step of measuring or monitoring the IGF-I standard deviation score (SDS) in a plasma or serum sample obtained from the patient during an initial dosage period of administration of an initial dose of human growth hormone-XTEN (hGH-XTEN) fusion protein. In another embodiment, the method further comprises the step of determining a subsequent dose of hGH-XTEN fusion protein administered over a subsequent dosage period based on the IGF-I SDS observed during the initial dosage period. In other embodiments, the determining step comprises determining a subsequent dosage period based upon the IGF-I SDS observed during the initial dosage period. In one additional embodiment, the subsequent dose and/or the subsequent dosing period improves the efficacy of the treatment during the subsequent dosage period.

In another aspect, the present invention provides a kit comprising a pharmaceutical composition, which comprises an hGH-XTEN fusion protein for the treatment of human GHD. In one embodiment, the hGH-XTEN fusion protein comprises (i) an amino acid sequence having at least about 90% sequence identity to SEQ ID NO:1; or (ii) the amino acid sequence of SEQ ID NO:1. In another embodiment, the kit comprises a container which holds a pharmaceutical composition comprising the hGH-XTEN fusion protein. In one additional embodiment, the kit further comprises a package insert associated with said container. In other embodiments, the package insert indicates that said composition is for the treatment of growth hormone deficiency by administration of an initial dose of the hGH-XTEN fusion protein and a plurality of subsequent doses of the hGH-XTEN fusion protein. In another embodiment, the initial dose and plurality of subsequent bolus doses each comprise a bolus dose. In one other embodiment, the bolus dose is a therapeutically effective bodyweight adjusted bolus dose. In one embodiment, the initial dose of the hGH-XTEN fusion protein is between about 0.05 mg/kg and about 3.0 mg/kg. In another embodiment, the plurality of subsequent doses of the hGH-XTEN fusion protein in between about 0.05 mg/kg and about 3.0 mg/kg. In one embodiment, the doses are administered once, every week, every two weeks, every three weeks, or monthly.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 provides the amino acid sequence for an hGH-XTEN fusion protein (hGH sequence is underlined and bold) (SEQ ID NO:1).

FIG. 2 summarizes the study phases of the VRS-317 Phase I study.

FIG. 3 summarizes the patient disposition.

FIG. 4 shows the human pharmacokinetic (PK) profile for various single doses of VRS-317.

FIG. 5 illustrates a dose response: change in mean IGF-I SDS for mg VRS-317/kg doses.

FIG. 6 illustrates a sustained IGF-I response to a single dose of VRS-317.

FIG. 7 summarizes adverse events reported after administration of various single doses of VRS-317.

### DESCRIPTION OF THE INVENTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### DEFINITIONS

As used herein, the following terms have the meanings ascribed to them unless specified otherwise.

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non amino acids. The terms also encompass an amino acid polymer that has been modified, for example, by disulfide bond formation, glycosylation, lipidation, acetylation, phosphorylation, or any other manipulation, such as conjugation with a labeling component.

As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The term “non-naturally occurring,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. A number of scales have been developed. An example is a scale developed by Levitt, M, et al., J Mol Biol (1976) 104:59, which is listed in Hopp, T P, et al., Proc Natl Acad Sci USA (1981) 78:3824. Examples of “hydrophilic amino acids” are arginine, lysine, threonine, alanine, asparagine, and glutamine. Of particular interest are the hydrophilic amino acids aspartate, glutamate, and serine, and glycine. Examples of “hydrophobic amino acids” are tryptophan, tyrosine, phenylalanine, methionine, leucine, isoleucine, and valine.

A “fragment” is a truncated form of a native biologically active protein that retains at least a portion of the therapeutic and/or biological activity. A “variant” is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity with the reference biologically active protein. As used herein, the term “biologically active protein moiety” includes proteins modified deliberately, as for example, by site directed mutagenesis, insertions, or accidentally through mutations.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

A “chimeric” protein contains at least one fusion polypeptide comprising regions in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Conjugated”, “linked,” “fused,” and “fusion” are used interchangeably herein. These terms refer to the joining together of two or more chemical elements or components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two or more segments that correspond to polypeptides encoded by the original ORFs (which segments are not normally so joined in nature).

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” or “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

“Ligation” refers to the process of forming phosphodiester bonds between two nucleic acid fragments or genes, linking them together. To ligate the DNA fragments or genes together, the ends of the DNA must be compatible with each other. In some cases, the ends will be directly compatible after endonuclease digestion. However, it may be necessary to first convert the staggered ends commonly produced after endonuclease digestion to blunt ends to make them compatible for ligation.

The terms “stringent conditions” or “stringent hybridization conditions” includes reference to conditions under which a polynucleotide will hybridize to its target sequence, to a detectably greater degree than other sequences (e.g., at least 2-fold over background). Generally, stringency of hybridization is expressed, in part, with reference to the temperature and salt concentration under which the wash step is carried out. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60° C. to 65° C. Alternatively, temperatures of about 65° C., 60° C., 55° C., or 42° C. may be used. SSC concentration may be varied from about 0.1 to 2×SSC, with SDS being present at about 0.1%. Such wash temperatures are typically selected to be about 5° C. to 20° C. lower than the thermal melting point for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al. (1989) Molecular Cloning: A Laboratory Manual, 2nd ed., vol. 1-3, Cold Spring Harbor Press, Plainview N.Y.; specifically see volume 2 and chapter 9. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) amino acid sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The term “non-repetitiveness” as used herein in the context of a polypeptide refers to a lack or limited degree of internal homology in a peptide or polypeptide sequence. The term “substantially non-repetitive” can mean, for example, that there are few or no instances of four contiguous amino acids in the sequence that are identical amino acid types or that the polypeptide has a subsequence score (defined infra) of 10 or less or that there isn't a pattern in the order, from N— to C-terminus, of the sequence motifs that constitute the polypeptide sequence. The term “repetitiveness” as used herein in the context of a polypeptide refers to the degree of internal homology in a peptide or polypeptide sequence. In contrast, a “repetitive” sequence may contain multiple identical copies of short amino acid sequences. For instance, a polypeptide sequence of interest may be divided into n-mer sequences and the number of identical sequences can be counted. Highly repetitive sequences contain a large fraction of identical sequences while non-repetitive sequences contain few identical sequences. In the context of a polypeptide, a sequence can contain multiple copies of shorter sequences of defined or variable length, or motifs, in which the motifs themselves have non-repetitive sequences, rendering the full-length polypeptide substantially non-repetitive. The length of polypeptide within which the non-repetitiveness is measured can vary from 3 amino acids to about 200 amino acids, about from 6 to about 50 amino acids, or from about 9 to about 14 amino acids.

“Repetitiveness” used in the context of polynucleotide sequences refers to the degree of internal homology in the sequence such as, for example, the frequency of identical nucleotide sequences of a given length. Repetitiveness can, for example, be measured by analyzing the frequency of identical sequences.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

“Serum degradation resistance,” as applied to a polypeptide, refers to the ability of the polypeptides to withstand degradation in blood or components thereof, which typically involves proteases in the serum or plasma. The serum degradation resistance can be measured by combining the protein with human (or mouse, rat, monkey, as appropriate) serum or plasma, typically for a range of days (e.g. 0.25, 0.5, 1, 2, 4, 8, 16 days), typically at about 37° C. The samples for these time points can be run on a Western blot assay and the protein is detected with an antibody. The antibody can be to a tag in the protein. If the protein shows a single band on the western, where the protein's size is identical to that of the injected protein, then no degradation has occurred. In this exemplary method, the time point where 50% of the protein is degraded, as judged by Western blots or equivalent techniques, is the serum degradation half-life or “serum half-life” of the protein.

The term “t½” as used herein means the terminal half-life calculated as ln(2)/Kₑₗ. Kₑₗ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. The terms “t½”, “terminal half-life”, “elimination half-life” and “circulating half-life” are used interchangeably herein.

“Apparent Molecular Weight Factor” or “Apparent Molecular Weight” are related terms referring to a measure of the relative increase or decrease in apparent molecular weight exhibited by a particular amino acid sequence. The Apparent Molecular Weight is determined using size exclusion chromatography (SEC) and similar methods compared to globular protein standards and is measured in “apparent kD” units. The Apparent Molecular Weight Factor is the ratio between the Apparent Molecular Weight and the actual molecular weight; the latter predicted by adding, based on amino acid composition, the calculated molecular weight of each type of amino acid in the composition.

The “hydrodynamic radius” or “Stokes radius” is the effective radius (Rh in nm) of a molecule in a solution measured by assuming that it is a body moving through the solution and resisted by the solution's viscosity. In the embodiments of the invention, the hydrodynamic radius measurements of the XTEN fusion proteins correlate with the ‘Apparent Molecular Weight Factor’, which is a more intuitive measure. The “hydrodynamic radius” of a protein affects its rate of diffusion in aqueous solution as well as its ability to migrate in gels of macromolecules. The hydrodynamic radius of a protein is determined by its molecular weight as well as by its structure, including shape and compactness. Methods for determining the hydrodynamic radius are well known in the art, such as by the use of size exclusion chromatography (SEC), as described in U.S. Pat. Nos. 6,406,632 and 7,294,513. Most proteins have globular structure, which is the most compact three-dimensional structure a protein can have with the smallest hydrodynamic radius. Some proteins adopt a random and open, unstructured, or ‘linear’ conformation and as a result have a much larger hydrodynamic radius compared to typical globular proteins of similar molecular weight.

“Physiological conditions” refer to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers is listed in Sambrook et al. (1989). Physiologically relevant temperature ranges from about 250 C to about 380 C, and preferably from about 350 C to about 370 C.

A “reactive group” is a chemical structure that can be coupled to a second reactive group. Examples for reactive groups are amino groups, carboxyl groups, sulfhydryl groups, hydroxyl groups, aldehyde groups, azide groups. Some reactive groups can be activated to facilitate coupling with a second reactive group. Non-limiting examples for activation are the reaction of a carboxyl group with carbodiimide, the conversion of a carboxyl group into an activated ester, or the conversion of a carboxyl group into an azide function.

“Controlled release agent”, “slow release agent”, “depot formulation” or “sustained release agent” are used interchangeably to refer to an agent capable of extending the duration of release of a polypeptide of the invention relative to the duration of release when the polypeptide is administered in the absence of agent. Different embodiments of the present invention may have different release rates, resulting in different therapeutic amounts.

The terms “antigen”, “target antigen” or “immunogen” are used interchangeably herein to refer to the structure or binding determinant that an antibody fragment or an antibody fragment-based therapeutic binds to or has specificity against.

The term “payload” as used herein refers to a protein or peptide sequence that has biological or therapeutic activity; the counterpart to the pharmacophore of small molecules. Examples of payloads include, but are not limited to, cytokines, enzymes, hormones and blood and growth factors. Payloads can further comprise genetically fused or chemically conjugated moieties such as chemotherapeutic agents, antiviral compounds, toxins, or contrast agents.

These conjugated moieties can be joined to the rest of the polypeptide via a linker that may be cleavable or non-cleavable.

The term “antagonist”, as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present invention, antagonists may include proteins, nucleic acids, carbohydrates, antibodies or any other molecules that decrease the effect of a biologically active protein.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

“Activity” for the purposes herein refers to an action or effect of a component of a fusion protein consistent with that of the corresponding native biologically active protein, wherein “biological activity” refers to an in vitro or in vivo biological function or effect, including but not limited to receptor binding, antagonist activity, agonist activity, or a cellular or physiologic response.

As used herein, “treatment” or “treating,” or “palliating” or “ameliorating” is used interchangeably herein. These terms refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect”, as used herein, refers to a physiologic effect, including but not limited to the cure, mitigation, amelioration, or prevention of disease in humans or other animals, or to otherwise enhance physical or mental wellbeing of humans or animals, caused by a fusion polypeptide of the invention other than the ability to induce the production of an antibody against an antigenic epitope possessed by the biologically active protein. Determination of a therapeutically effective amount is well within the capability of those skilled in the art, especially in light of the detailed disclosure provided herein.

The terms “therapeutically effective amount” and “therapeutically effective dose”, as used herein, refers to an amount of a biologically active protein, either alone or as a part of a fusion protein composition, that is capable of having any detectable, beneficial effect on any symptom, aspect, measured parameter or characteristics of a disease state or condition when administered in one or repeated doses to a subject. Such effect need not be absolute to be beneficial.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

The term “therapeutically effective dose regimen”, as used herein, refers to a schedule for consecutively administered doses of a biologically active protein, either alone or as a part of a fusion protein composition, wherein the doses are given in therapeutically effective amounts to result in sustained beneficial effect on any symptom, aspect, measured parameter or characteristics of a disease state or condition.

### I). General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3rd edition, Cold Spring Harbor Laboratory Press, 2001; “Current protocols in molecular biology”, F. M. Ausubel, et al. eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11 th Edition, McGraw-Hill, 2005; and Freshney, R. I., “Culture of Animal Cells: A Manual of Basic Technique,” 4th edition, John Wiley & Sons, Somerset, N.J., 2000, the contents of which are incorporated in their entirety herein by reference.

### II). Growth Hormone

The present invention concerns an improved therapeutic regimen for GHD therapy. In particular, the invention concerns methods for bolus dose administration of a hGH-XTEN fusion protein to a patient with GHD. Accordingly, in one aspect, the present invention concerns a method of treating human growth hormone deficiency (GHD) with a hGH-XTEN fusion protein.

(a) Growth Hormone Proteins

“Growth Hormone” or “GH” means a growth hormone protein and species and sequence variants thereof, and includes, but is not limited to, the 191 single-chain amino acid sequence of human GH. The GH can be the native, full-length protein or can be a truncated fragment or a sequence variant that retains at least a portion of the biological activity of the native protein. There are two known types of human GH (hereinafter “hGH”) derived from the pituitary gland: one having a molecular weight of about 22,129 daltons (22 kD hGH) and the other having a molecular weight of about 20,000 daltons (20 kD hGH). The 20 kD HGH has an amino acid sequence that corresponds to that of 22 kD hGH consisting of 191 amino acids except that 15 amino acid residues from the 32nd to the 46th of 22 kD hGH are missing. Some reports have shown that the 20 kD hGH has been found to exhibit lower risks and higher activity than 22 kD hGH. The invention contemplates use of the 22 kD, the 20 kD hGH, as well as species and sequence variants and truncated fragments thereof as being appropriate for use as a fusion partner with XTEN disclosed herein for hGH-XTEN compositions. The cloned gene for hGH has been expressed in a secreted form in Escherichia coli (U.S. Pat. No. 4,898,830; Chang, C. N., et al., Gene 55:189 [1987]) and its DNA and amino acid sequence has been reported (Goeddel, et al. Nature, 281:544 [1979]); Gray, et al., Gene 39: 247[1985]).

The invention contemplates inclusion in the hGH-XTEN compositions sequences with homology to GH sequences, sequence fragments that are natural, such as from humans and non-natural sequence variants which retain at least a portion of the biologic activity or biological function of GH and/or that are useful for preventing, treating, mediating, or ameliorating a GH-related disease, deficiency, disorder or condition. In addition, native sequences homologous to human GH may be found by standard homology searching techniques, such as NCBI BLAST.

Effects of GH on the tissues of the body can generally be described as anabolic. Like most other protein hormones, native GH acts by interacting with a specific plasma membrane receptor, referred to as growth hormone receptor. GH acts on the liver and other tissues to stimulate production of IGF-I, which is responsible for the growth promoting effects of GH and also reflects the amount produced. IGF-I, in turn, has stimulatory effects on osteoblast and chondrocyte activity to promote bone growth. In one embodiment, the invention provides a hGH-XTEN that exhibits at least one of the properties of native GH hereinabove described herein.

In one embodiment, the GH incorporated into the subject compositions is a recombinant polypeptide with a sequence corresponding to a protein found in nature. In another embodiment, the GH is a sequence variant, fragment, homolog, or a mimetics of a natural sequence that retains at least a portion of the biological activity of the corresponding native GH. In one other embodiment, the GH is human GH comprising the following amino acid sequence: FPTIPLSRLFDNAMLRAHRLHQLAFDTYQEFEEAYIPKEQKYSFLQNPQTSLCFSESIPTP SNREETQQKSNLELLRISLLLIQSWLEPVQFLRSVFANSLVYGASDSNVYDLLKDLEEGI QTLMGRLEDGSPRTGQIFKQTYSKFDTNSHNDDALLKNYGLLYCFRKDMDKVETFLRI VQCRSVEGSCGF (SEQ ID NO:2). Any human GH sequences or homologous derivatives constructed by shuffling individual mutations between families that retain at least a portion of the biological activity of the native GH may be useful for the fusion proteins of this invention. GH that can be incorporated into a hGH-XTEN fusion protein can include a protein that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:2.

### III). Human Growth Hormone-XTEN Fusion Protein Compositions For Treating GHD

The present invention concerns an improved therapeutic regimen for growth hormone deficiency (GHD) therapy. In particular, the invention concerns methods for bolus dose administration of hGH-XTEN fusion proteins to a patient with GHD. In one aspect, the hGH fusion proteins suitable for use in the present invention comprise a human growth hormone polypeptide and one or more XTEN sequences as described herein, and as disclosed in Schellenberger et al. WO10/144,502A2 and WO10/091,122, which are incorporated herein by reference in their entirety.

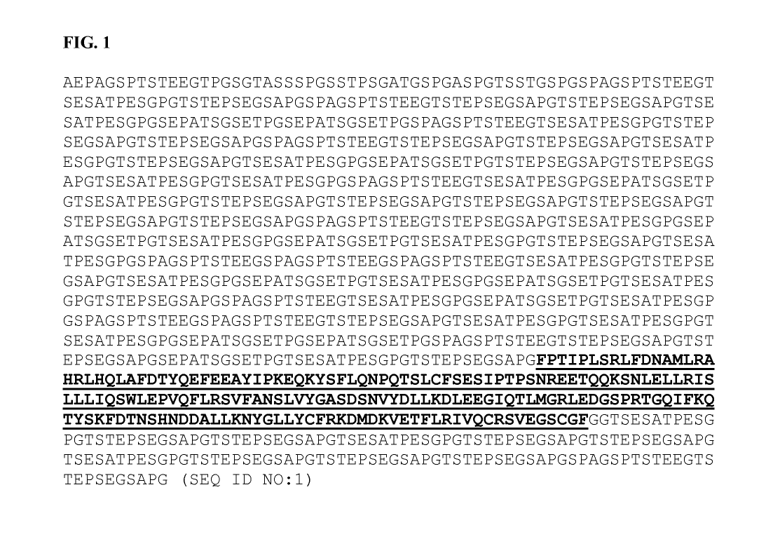

In one other aspect, the hGH-XTEN fusion proteins are isolated monomeric fusion proteins of GH comprising the full-length sequence or sequence variants of GH covalently linked to one or more extended recombinant polypeptides (“XTEN” or “XTENs”). In one embodiment, the hGH-XTEN fusion protein comprises an amino acid sequence shown in FIG. 1 (SEQ ID NO: 1), or pharmacologically active variants thereof. In another embodiment, the hGH-XTEN fusion protein comprises an amino acid sequence selected from Table 1.

The fusion protein VRS-317, is composed of recombinant human growth hormone (rhGH) and two recombinant polypeptides, referred to as XTEN as described in Schellenberger et al. (2009). Nat Biotechnol 27, 1186-90, Schellenberger et al. WO10/144,502A2, and WO10/091122, each of which are incorporated herein by reference in their entirety. The XTEN domain, two unstructured hydrophilic chains of amino acids, provides half-life extension for rhGH. The molecular weight of VRS-317 is 118.9 kDa, with rhGH contributing 22.1 kDa and the remaining mass contributed by the XTEN construct. The mass ratio of rhGH to VRS-317 is therefore 1:5.37. The amino acid sequence of the VRS-317 fusion protein is provided in FIG. 1.

TABLE 1
Exemplary hGH-XTEN fusion proteins

| hGH- | | SEQ | | SEQ |
|---|---|---|---|---|
| XTEN | Amino Acid | ID | | ID |
| Name* | Sequence | NO: | DNA NucleotideSequence | NO: |
| AM864- | GGSPGTSTEPSEGSAPG | 3 | ggtGGGTCTCCAGGTACTTCTACTGAACCGTCTG | 4 |
| hGH | SEPATSGSETPGSPAGSP | | AAGGCAGCGCACCAGGTAGCGAACCGGCTACT | |
| | TSTEEGSTSSTAESPGPG | | TCCGGTTCTGAAACCCCAGGTAGCCCAGCAGGT | |
| | TSTPESGSASPGSTSESP | | TCTCCAACTTCTACTGAAGAAGGTTCTACCAGC | |
| | SGTAPGSTSESPSGTAP | | TCTACCGCAGAATCTCCTGGTCCAGGTACCTCT | |
| | GTSTPESGSASPGTSTPE | | ACTCCGGAAAGCGGCTCTGCATCTCCAGGTTCT | |
| | SGSASPGSEPATSGSETP | | ACTAGCGAATCTCCTTCTGGCACTGCACCAGGT | |
| | GTSESATPESGPGSPAG | | TCTACTAGCGAATCCCCGTCTGGTACTGCTCCA | |
| | SPTSTEEGTSTEPSEGSA | | GGTACTTCTACTCCTGAAAGCGGTTCCGCTTCTC | |
| | PGTSESATPESGPGTSTE | | CAGGTACCTCTACTCCGGAAAGCGGTTCTGCAT | |
| | PSEGSAPGTSTEPSEGS | | CTCCAGGTAGCGAACCGGCAACCTCCGGCTCTG | |
| | APGSPAGSPTSTEEGTS | | AAACCCCAGGTACCTCTGAAAGCGCTACTCCTG | |
| | TEPSEGSAPGTSTEPSEG | | AATCCGGCCCAGGTAGCCCGGCAGGTTCTCCGA | |
| | SAPGTSESATPESGPGT | | CTTCCACTGAGGAAGGTACCTCTACTGAACCTT | |
| | SESATPESGPGTSTEPSE | | CTGAGGGCAGCGCTCCAGGTACTTCTGAAAGCG | |
| | GSAPGTSTEPSEGSAPG | | CTACCCCGGAGTCCGGTCCAGGTACTTCTACTG | |
| | TSESATPESGPGTSTEPS | | AACCGTCCGAAGGTAGCGCACCAGGTACTTCTA | |
| | EGSAPGSEPATSGSETP | | CCGAACCGTCCGAGGGTAGCGCACCAGGTAGC | |
| | GSPAGSPTSTEEGSSTPS | | CCAGCAGGTTCTCCTACCTCCACCGAGGAAGGT | |
| | GATGSPGTPGSGTASSS | | ACTTCTACCGAACCGTCCGAGGGTAGCGCACCA | |
| | PGSSTPSGATGSPGTST | | GGTACTTCTACCGAACCTTCCGAGGGCAGCGCA | |
| | EPSEGSAPGTSTEPSEGS | | CCAGGTACTTCTGAAAGCGCTACCCCTGAGTCC | |
| | APGSEPATSGSETPGSP | | GGCCCAGGTACTTCTGAAAGCGCTACTCCTGAA | |
| | AGSPTSTEEGSPAGSPT | | TCCGGTCCAGGTACCTCTACTGAACCTTCCGAA | |
| | STEEGTSTEPSEGSAPG | | GGCAGCGCTCCAGGTACCTCTACCGAACCGTCC | |
| | ASASGAPSTGGTSESAT | | GAGGGCAGCGCACCAGGTACTTCTGAAAGCGC | |
| | PESGPGSPAGSPTSTEE | | AACCCCTGAATCCGGTCCAGGTACTTCTACTGA | |
| | GSPAGSPTSTEEGSTSST | | ACCTTCCGAAGGTAGCGCTCCAGGTAGCGAACC | |
| | AESPGPGSTSESPSGTAP | | TGCTACTTCTGGTTCTGAAACCCCAGGTAGCCC | |
| | GTSPSGESSTAPGTPGS | | GGCTGGCTCTCCGACCTCCACCGAGGAAGGTAG | |
| | GTASSSPGSSTPSGATG | | CTCTACCCCGTCTGGTGCTACTGGTTCTCCAGGT | |
| | SPGSSPSASTGTGPGSEP | | ACTCCGGGCAGCGGTACTGCTTCTTCCTCTCCA | |
| | ATSGSETPGTSESATPES | | GGTAGCTCTACCCCTTCTGGTGCTACTGGCTCTC | |
| | GPGSEPATSGSETPGST | | CAGGTACCTCTACCGAACCGTCCGAGGGTAGCG | |
| | SSTAESPGPGSTSSTAES | | CACCAGGTACCTCTACTGAACCGTCTGAGGGTA | |
| | PGPGTSPSGESSTAPGSE | | GCGCTCCAGGTAGCGAACCGGCAACCTCCGGTT | |
| | PATSGSETPGSEPATSG | | CTGAAACTCCAGGTAGCCCTGCTGGCTCTCCGA | |
| | SETPGTSTEPSEGSAPGS | | CTTCTACTGAGGAAGGTAGCCCGGCTGGTTCTC | |
| | TSSTAESPGPGTSTPESG | | CGACTTCTACTGAGGAAGGTACTTCTACCGAAC | |
| | SASPGSTSESPSGTAPGT | | CTTCCGAAGGTAGCGCTCCAGGTGCAAGCGCAA | |
| | STEPSEGSAPGTSTEPSE | | GCGGCGCGCCAAGCACGGGAGGTACTTCTGAA | |
| | GSAPGTSTEPSEGSAPG | | AGCGCTACTCCTGAGTCCGGCCCAGGTAGCCCG | |
| | SSTPSGATGSPGSSPSAS | | GCTGGCTCTCCGACTTCCACCGAGGAAGGTAGC | |
| | TGTGPGASPGTSSTGSP | | CCGGCTGGCTCTCCAACTTCTACTGAAGAAGGT | |
| | GSEPATSGSETPGTSES | | TCTACCAGCTCTACCGCTGAATCTCCTGGCCCA | |
| | ATPESGPGSPAGSPTST | | GGTTCTACTAGCGAATCTCCGTCTGGCACCGCA | |
| | EEGSSTPSGATGSPGSSP | | CCAGGTACTTCCCCTAGCGGTGAATCTTCTACT | |
| | SASTGTGPGASPGTSST | | GCACCAGGTACCCCTGGCAGCGGTACCGCTTCT | |
| | GSPGTSESATPESGPGT | | TCCTCTCCAGGTAGCTCTACCCCGTCTGGTGCTA | |
| | STEPSEGSAPGTSTEPSE | | CTGGCTCTCCAGGTTCTAGCCCGTCTGCATCTAC | |
| | GSAPGFPTIPLSRLFDNA | | CGGTACCGGCCCAGGTAGCGAACCGGCAACCT | |
| | MLRAHRLHQLAFDTYQ | | CCGGCTCTGAAACTCCAGGTACTTCTGAAAGCG | |
| | EFEEAYIPKEQKYSFLQ | | CTACTCCGGAATCCGGCCCAGGTAGCGAACCGG | |
| | NPQTSLCFSESIPTPSNR | | CTACTTCCGGCTCTGAAACCCCAGGTTCCACCA | |
| | EETQQKSNLELLRISLL | | GCTCTACTGCAGAATCTCCGGGCCCAGGTTCTA | |
| | LIQSWLEPVQFLRSVFA | | CTAGCTCTACTGCAGAATCTCCGGGTCCAGGTA | |
| | NSLVYGASDSNVYDLL | | CTTCTCCTAGCGGCGAATCTTCTACCGCTCCAG | |
| | KDLEEGIQTLMGRLED | | GTAGCGAACCGGCAACCTCTGGCTCTGAAACTC | |
| | GSPRTGQIFKQTYSKFD | | CAGGTAGCGAACCTGCAACCTCCGGCTCTGAAA | |
| | TNSHNDDALLKNYGLL | | CCCCAGGTACTTCTACTGAACCTTCTGAGGGCA | |
| | YCFRKDMDKVETFLRI | | GCGCACCAGGTTCTACCAGCTCTACCGCAGAAT | |
| | VQCRSVEGSCGF | | CTCCTGGTCCAGGTACCTCTACTCCGGAAAGCG | |
| | | | GCTCTGCATCTCCAGGTTCTACTAGCGAATCTC | |
| | | | CTTCTGGCACTGCACCAGGTACTTCTACCGAAC | |
| | | | CGTCCGAAGGCAGCGCTCCAGGTACCTCTACTG | |
| | | | AACCTTCCGAGGGCAGCGCTCCAGGTACCTCTA | |
| | | | CCGAACCTTCTGAAGGTAGCGCACCAGGTAGCT | |
| | | | CTACTCCGTCTGGTGCAACCGGCTCCCCAGGTT | |
| | | | CTAGCCCGTCTGCTTCCACTGGTACTGGCCCAG | |
| | | | GTGCTTCCCCGGGCACCAGCTCTACTGGTTCTC | |
| | | | CAGGTAGCGAACCTGCTACCTCCGGTTCTGAAA | |
| | | | CCCCAGGTACCTCTGAAAGCGCAACTCCGGAGT | |
| | | | CTGGTCCAGGTAGCCCTGCAGGTTCTCCTACCT | |
| | | | CCACTGAGGAAGGTAGCTCTACTCCGTCTGGTG | |
| | | | CAACCGGCTCCCCAGGTTCTAGCCCGTCTGCTT | |
| | | | CCACTGGTACTGGCCCAGGTGCTTCCCCGGGCA | |
| | | | CCAGCTCTACTGGTTCTCCAGGTACCTCTGAAA | |
| | | | GCGCTACTCCGGAGTCTGGCCCAGGTACCTCTA | |
| | | | CTGAACCGTCTGAGGGTAGCGCTCCAGGTACTT | |
| | | | CTACTGAACCGTCCGAAGGTAGCGCACCAGGTT | |
| | | | TTCCGACTATTCCGCTGTCTCGTCTGTTTGATAA | |
| | | | TGCTATGCTGCGTGCGCACCGTCTGCACCAGCT | |
| | | | GGCCTTTGATACTTACCAGGAATTTGAAGAAGC | |
| | | | cTACATTCCTAAAGAGCAGAAGTACTCTTTCCTG | |
| | | | CAAAACCCACAGACTTCTCTCTGCTTCAGCGAA | |
| | | | TCTATTCCGACGCCTTCCAATCGCGAGGAAACT | |
| | | | CAGCAAAAGTCCAATCTGGAACTACTCCGCATT | |
| | | | TCTCTGCTTCTGATTCAGAGCTGGCTAGAACCA | |
| | | | GTGCAATTTCTGCGTTCCGTCTTCGCCAATAGCC | |
| | | | TAGTTTATGGCGCATCCGACAGCAACGTATACG | |
| | | | ATCTCCTGAAAGATCTCGAGGAAGGCATTCAGA | |
| | | | CCCTGATGGGTCGTCTCGAGGATGGCTCTCCGC | |
| | | | GTACTGGTCAGATCTTCAAGCAGACTTACTCTA | |
| | | | AATTTGATACTAACAGCCACAATGACGATGCGC | |
| | | | TTCTAAAAAACTATGGTCTGCTGTATTGTTTTCG | |
| | | | TAAAGATATGGACAAAGTTGAAACCTTCCTGCG | |
| | | | TATTGTTCAGTGTCGTTCCGTTGAGGGCAGCTG | |
| | | | TGGTTTC | |
| Y576- | GEGSGEGSEGEGSEGSG | 5 | GGTGAGGGTTCTGGCGAAGGTTCCGAAGGTGA | 6 |
| hGH | EGEGSEGSGEGEGGSE | | GGGCTCCGAAGGATCTGGCGAAGGTGAGGGTT | |
| | GSEGEGSEGSGEGEGG | | CCGAAGGTTCTGGCGAAGGTGAAGGCGGTTCTG | |
| | EGSGEGEGSGEGSEGE | | AGGGATCCGAAGGTGAAGGCTCCGAAGGATCT | |
| | GGGEGSEGEGSGEGGE | | GGCGAAGGTGAAGGTGGTGAAGGTTCTGGCGA | |
| | GEGSEGGSEGEGGSEG | | AGGTGAGGGATCTGGCGAAGGCTCTGAAGGTG | |
| | GEGEGSEGSGEGEGSE | | AAGGTGGTGGTGAAGGCTCTGAAGGTGAAGGA | |
| | GGSEGEGSEGGSEGEGS | | TCTGGTGAAGGTGGCGAAGGTGAGGGATCTGA | |
| | EGSGEGEGSEGSGEGE | | AGGCGGCTCCGAAGGTGAAGGCGGATCTGAAG | |
| | GSEGSGEGEGSEGSGEG | | GCGGCGAAGGTGAAGGTTCCGAAGGTTCTGGT | |
| | EGSEGGSEGEGGSEGSE | | GAAGGTGAAGGATCTGAAGGTGGCTCCGAAGG | |
| | GEGSGEGSEGEGGSEGS | | TGAAGGATCTGAAGGCGGTTCCGAAGGTGAGG | |
| | EGEGGGEGSEGEGSGE | | GCTCTGAAGGTTCTGGCGAAGGTGAAGGCTCTG | |
| | GSEGEGGSEGSEGEGGS | | AAGGATCTGGTGAAGGTGAAGGTTCCGAAGGT | |
| | EGSEGEGGEGSGEGEG | | TCTGGTGAAGGTGAAGGTTCCGAAGGTTCTGGC | |
| | SEGSGEGEGSGEGSEGE | | GAAGGTGAAGGTTCTGAAGGTGGCTCTGAAGG | |
| | GSEGSGEGEGSEGSGEG | | TGAAGGCGGCTCTGAAGGATCCGAAGGTGAAG | |
| | EGGSEGSEGEGSGEGSE | | GTTCTGGTGAAGGCTCTGAAGGTGAAGGCGGCT | |
| | GEGSEGSGEGEGSEGSG | | CTGAGGGTTCCGAAGGTGAAGGCGGAGGCGAA | |
| | EGEGGSEGSEGEGGSE | | GGTTCTGAAGGTGAGGGATCTGGTGAAGGTTCT | |
| | GSEGEGGSEGSEGEGG | | GAAGGTGAAGGCGGTTCTGAAGGTTCCGAAGG | |
| | EGSGEGEGSEGSGEGE | | TGAAGGTGGCTCTGAGGGATCCGAAGGTGAAG | |
| | GSGEGSEGEGSEGSGEG | | GTGGCGAAGGATCTGGTGAAGGTGAAGGTTCT | |
| | EGSEGSGEGEGGSEGSE | | GAAGGTTCTGGCGAAGGTGAGGGTTCTGGCGA | |
| | GEGSEGSGEGEGGEGS | | AGGTTCCGAAGGTGAGGGCTCCGAAGGATCTG | |
| | GEGEGSGEGSEGEGGG | | GCGAAGGTGAGGGTTCCGAAGGTTCTGGCGAA | |
| | EGSEGEGSEGSGEGEGS | | GGTGAAGGCGGTTCTGAGGGATCCGAAGGTGA | |
| | EGSGEGEGSEGGSEGE | | GGGTTCTGGCGAAGGTTCCGAAGGTGAGGGCTC | |
| | GGSEGSEGEGSEGGSEG | | CGAAGGATCTGGCGAAGGTGAGGGTTCCGAAG | |
| | EGSEGGSEGEGSEGSGE | | GTTCTGGCGAAGGTGAAGGCGGTTCTGAGGGAT | |
| | GEGSEGSGEGEGSGEGS | | CCGAAGGTGAAGGCGGTTCTGAAGGTTCCGAA | |
| | EGEGGSEGGEGEGSEG | | GGTGAAGGTGGCTCTGAGGGATCCGAAGGTGA | |
| | GSEGEGSEGGSEGEGG | | AGGTGGCGAAGGATCTGGTGAAGGTGAAGGTT | |
| | EGSGEGEGGGEGSEGE | | CTGAAGGTTCTGGCGAAGGTGAGGGTTCTGGCG | |
| | GSEGSGEGEGSGEGSEG | | AAGGTTCCGAAGGTGAGGGCTCCGAAGGATCT | |
| | FPTIPLSRLFDNAMLRA | | GGCGAAGGTGAGGGTTCCGAAGGTTCTGGCGA | |
| | HRLHQLAFDTYQEFEE | | AGGTGAAGGCGGTTCTGAGGGATCCGAAGGTG | |
| | AYIPKEQKYSFLQNPQT | | AAGGCTCCGAAGGATCTGGCGAAGGTGAAGGT | |
| | SLCFSESIPTPSNREETQ | | GGTGAAGGTTCTGGCGAAGGTGAGGGATCTGG | |
| | QKSNLELLRISLLLIQS | | CGAAGGCTCTGAAGGTGAAGGTGGTGGTGAAG | |
| | WLEPVQFLRSVFANSL | | GCTCTGAAGGTGAAGGTTCCGAAGGTTCTGGTG | |
| | VYGASDSNVYDLLKDL | | AAGGTGAAGGTTCCGAAGGTTCTGGCGAAGGT | |
| | EEGIQTLMGRLEDGSPR | | GAAGGTTCTGAAGGTGGCTCTGAAGGTGAAGG | |
| | TGQIFKQTYSKFDTNSH | | CGGCTCTGAAGGATCCGAAGGTGAAGGATCTG | |
| | NDDALLKNYGLLYCFR | | AAGGTGGCTCCGAAGGTGAAGGATCTGAAGGC | |
| | KDMDKVETFLRIVQCR | | GGTTCCGAAGGTGAGGGCTCTGAAGGTTCTGGC | |
| | SVEGSCGF | | GAAGGTGAAGGCTCTGAAGGATCTGGTGAAGG | |
| | | | TGAAGGATCTGGCGAAGGCTCCGAAGGTGAAG | |
| | | | GCGGTTCTGAAGGTGGCGAAGGTGAAGGATCT | |
| | | | GAAGGTGGTTCCGAAGGTGAGGGATCTGAAGG | |
| | | | TGGCTCTGAAGGTGAAGGTGGCGAAGGTTCTGG | |
| | | | CGAAGGTGAAGGTGGAGGCGAAGGTTCTGAAG | |
| | | | GTGAAGGTTCCGAAGGTTCTGGTGAAGGTGAG | |
| | | | GGATCTGGCGAAGGTTCTGAAGGTTTTCCGACT | |
| | | | ATTCCGCTGTCTCGTCTGTTTGATAACGCTATGC | |
| | | | TGCGTGCGCACCGTCTGCACCAGCTGGCGTTCG | |
| | | | ACACTTACCAGGAATTTGAAGAAGCGTACATTC | |
| | | | CGAAGGAACAGAAGTACTCTTTCCTGCAAAACC | |
| | | | CGCAGACCTCCCTGTGCTTCAGCGAATCTATTC | |
| | | | CGACTCCGTCCAATCGTGAAGAAACTCAGCAAA | |
| | | | AGTCCAATCTGGAGCTGCTGCGCATCTCTCTGC | |
| | | | TGCTGATTCAGAGCTGGCTGGAGCCTGTTCAGT | |
| | | | TTCTGCGTTCCGTCTTCGCCAACAGCCTGGTTTA | |
| | | | TGGTGCTTCCGACAGCAACGTATACGATCTGCT | |
| | | | GAAAGATCTGGAAGAAGGCATTCAGACCCTGA | |
| | | | TGGGTCGTCTGGAAGATGGTTCTCCGCGTACTG | |
| | | | GTCAGATCTTCAAACAAACTTACTCCAAATTTG | |
| | | | ATACTAACAGCCATAACGACGATGCTCTGCTGA | |
| | | | AAAACTATGGTCTGCTGTATTGCTTCCGCAAGG | |
| | | | ATATGGACAAAGTTGAAACCTTCCTGCGTATTG | |
| | | | TGCAGTGTCGTTCCGTTGAGGGCAGCTGTGGTT | |
| | | | TC | |
| AE912- | AEPAGSPTSTEEGTPGS | 7 | ATGGCTGAACCTGCTGGCTCTCCAACCTCCACT | 8 |
| hGH | GTASSSPGSSTPSGATG | | GAGGAAGGTACCCCGGGTAGCGGTACTGCTTCT | |
| | SPGASPGTSSTGSPGSP | | TCCTCTCCAGGTAGCTCTACCCCTTCTGGTGCAA | |
| | AGSPTSTEEGTSESATP | | CCGGCTCTCCAGGTGCTTCTCCGGGCACCAGCT | |
| | ESGPGTSTEPSEGSAPG | | CTACCGGTTCTCCAGGTAGCCCGGCTGGCTCTC | |
| | SPAGSPTSTEEGTSTEPS | | CTACCTCTACTGAGGAAGGTACTTCTGAAAGCG | |
| | EGSAPGTSTEPSEGSAP | | CTACTCCTGAGTCTGGTCCAGGTACCTCTACTG | |
| | GTSESATPESGPGSEPA | | AACCGTCCGAAGGTAGCGCTCCAGGTAGCCCA | |
| | TSGSETPGSEPATSGSET | | GCAGGCTCTCCGACTTCCACTGAGGAAGGTACT | |
| | PGSPAGSPTSTEEGTSES | | TCTACTGAACCTTCCGAAGGCAGCGCACCAGGT | |
| | ATPESGPGTSTEPSEGS | | ACCTCTACTGAACCTTCTGAGGGCAGCGCTCCA | |
| | APGTSTEPSEGSAPGSP | | GGTACTTCTGAAAGCGCTACCCCGGAATCTGGC | |
| | AGSPTSTEEGTSTEPSE | | CCAGGTAGCGAACCGGCTACTTCTGGTTCTGAA | |
| | GSAPGTSTEPSEGSAPG | | ACCCCAGGTAGCGAACCGGCTACCTCCGGTTCT | |
| | TSESATPESGPGTSTEPS | | GAAACTCCAGGTAGCCCGGCAGGCTCTCCGACC | |
| | EGSAPGTSESATPESGP | | TCTACTGAGGAAGGTACTTCTGAAAGCGCAACC | |
| | GSEPATSGSETPGTSTEP | | CCGGAGTCCGGCCCAGGTACCTCTACCGAACCG | |
| | SEGSAPGTSTEPSEGSA | | TCTGAGGGCAGCGCACCAGGTACTTCTACCGAA | |
| | PGTSESATPESGPGTSES | | CCGTCCGAGGGTAGCGCACCAGGTAGCCCAGC | |
| | ATPESGPGSPAGSPTST | | AGGTTCTCCTACCTCCACCGAGGAAGGTACTTC | |
| | EEGTSESATPESGPGSEP | | TACCGAACCGTCCGAGGGTAGCGCACCAGGTA | |
| | ATSGSETPGTSESATPES | | CCTCTACTGAACCTTCTGAGGGCAGCGCTCCAG | |
| | GPGTSTEPSEGSAPGTS | | GTACTTCTGAAAGCGCTACCCCGGAGTCCGGTC | |
| | TEPSEGSAPGTSTEPSEG | | CAGGTACTTCTACTGAACCGTCCGAAGGTAGCG | |
| | SAPGTSTEPSEGSAPGT | | CACCAGGTACTTCTGAAAGCGCAACCCCTGAAT | |
| | STEPSEGSAPGTSTEPSE | | CCGGTCCAGGTAGCGAACCGGCTACTTCTGGCT | |
| | GSAPGSPAGSPTSTEEG | | CTGAGACTCCAGGTACTTCTACCGAACCGTCCG | |
| | TSTEPSEGSAPGTSESAT | | AAGGTAGCGCACCAGGTACTTCTACTGAACCGT | |
| | PESGPGSEPATSGSETP | | CTGAAGGTAGCGCACCAGGTACTTCTGAAAGCG | |
| | GTSESATPESGPGSEPA | | CAACCCCGGAATCCGGCCCAGGTACCTCTGAAA | |
| | TSGSETPGTSESATPESG | | GCGCAACCCCGGAGTCCGGCCCAGGTAGCCCTG | |
| | PGTSTEPSEGSAPGTSES | | CTGGCTCTCCAACCTCCACCGAAGAAGGTACCT | |
| | ATPESGPGSPAGSPTST | | CTGAAAGCGCAACCCCTGAATCCGGCCCAGGTA | |
| | EEGSPAGSPTSTEEGSP | | GCGAACCGGCAACCTCCGGTTCTGAAACCCCAG | |
| | AGSPTSTEEGTSESATP | | GTACCTCTGAAAGCGCTACTCCGGAGTCTGGCC | |
| | ESGPGTSTEPSEGSAPG | | CAGGTACCTCTACTGAACCGTCTGAGGGTAGCG | |
| | TSESATPESGPGSEPATS | | CTCCAGGTACTTCTACTGAACCGTCCGAAGGTA | |
| | GSETPGTSESATPESGP | | GCGCACCAGGTACTTCTACCGAACCGTCCGAAG | |
| | GSEPATSGSETPGTSES | | GCAGCGCTCCAGGTACCTCTACTGAACCTTCCG | |
| | ATPESGPGTSTEPSEGS | | AGGGCAGCGCTCCAGGTACCTCTACCGAACCTT | |
| | APGSPAGSPTSTEEGTS | | CTGAAGGTAGCGCACCAGGTACTTCTACCGAAC | |
| | ESATPESGPGSEPATSG | | CGTCCGAGGGTAGCGCACCAGGTAGCCCAGCA | |
| | SETPGTSESATPESGPGS | | GGTTCTCCTACCTCCACCGAGGAAGGTACTTCT | |
| | PAGSPTSTEEGSPAGSP | | ACCGAACCGTCCGAGGGTAGCGCACCAGGTAC | |
| | TSTEEGTSTEPSEGSAP | | CTCTGAAAGCGCAACTCCTGAGTCTGGCCCAGG | |
| | GTSESATPESGPGTSES | | TAGCGAACCTGCTACCTCCGGCTCTGAGACTCC | |
| | ATPESGPGTSESATPES | | AGGTACCTCTGAAAGCGCAACCCCGGAATCTGG | |
| | GPGSEPATSGSETPGSE | | TCCAGGTAGCGAACCTGCAACCTCTGGCTCTGA | |
| | PATSGSETPGSPAGSPTS | | AACCCCAGGTACCTCTGAAAGCGCTACTCCTGA | |
| | TEEGTSTEPSEGSAPGT | | ATCTGGCCCAGGTACTTCTACTGAACCGTCCGA | |
| | STEPSEGSAPGSEPATS | | GGGCAGCGCACCAGGTACTTCTGAAAGCGCTAC | |
| | GSETPGTSESATPESGP | | TCCTGAGTCCGGCCCAGGTAGCCCGGCTGGCTC | |
| | GTSTEPSEGSAPGFPTIP | | TCCGACTTCCACCGAGGAAGGTAGCCCGGCTGG | |
| | LSRLFDNAMLRAHRLH | | CTCTCCAACTTCTACTGAAGAAGGTAGCCCGGC | |
| | QLAFDTYQEFEEAYIPK | | AGGCTCTCCGACCTCTACTGAGGAAGGTACTTC | |
| | EQKYSFLQNPQTSLCFS | | TGAAAGCGCAACCCCGGAGTCCGGCCCAGGTA | |
| | ESIPTPSNREETQQKSNL | | CCTCTACCGAACCGTCTGAGGGCAGCGCACCAG | |
| | ELLRISLLLIQSWLEPVQ | | GTACCTCTGAAAGCGCAACTCCTGAGTCTGGCC | |
| | FLRSVFANSLVYGASDS | | CAGGTAGCGAACCTGCTACCTCCGGCTCTGAGA | |
| | NVYDLLKDLEEGIQTL | | CTCCAGGTACCTCTGAAAGCGCAACCCCGGAAT | |
| | MGRLEDGSPRTGQIFK | | CTGGTCCAGGTAGCGAACCTGCAACCTCTGGCT | |
| | QTYSKFDTNSHNDDAL | | CTGAAACCCCAGGTACCTCTGAAAGCGCTACTC | |
| | LKNYGLLYCFRKDMD | | CTGAATCTGGCCCAGGTACTTCTACTGAACCGT | |
| | KVETFLRIVQCRSVEGS | | CCGAGGGCAGCGCACCAGGTAGCCCTGCTGGCT | |
| | CGF | | CTCCAACCTCCACCGAAGAAGGTACCTCTGAAA | |
| | | | GCGCAACCCCTGAATCCGGCCCAGGTAGCGAA | |
| | | | CCGGCAACCTCCGGTTCTGAAACCCCAGGTACT | |
| | | | TCTGAAAGCGCTACTCCTGAGTCCGGCCCAGGT | |
| | | | AGCCCGGCTGGCTCTCCGACTTCCACCGAGGAA | |
| | | | GGTAGCCCGGCTGGCTCTCCAACTTCTACTGAA | |
| | | | GAAGGTACTTCTACCGAACCTTCCGAGGGCAGC | |
| | | | GCACCAGGTACTTCTGAAAGCGCTACCCCTGAG | |
| | | | TCCGGCCCAGGTACTTCTGAAAGCGCTACTCCT | |
| | | | GAATCCGGTCCAGGTACTTCTGAAAGCGCTACC | |
| | | | CCGGAATCTGGCCCAGGTAGCGAACCGGCTACT | |
| | | | TCTGGTTCTGAAACCCCAGGTAGCGAACCGGCT | |
| | | | ACCTCCGGTTCTGAAACTCCAGGTAGCCCAGCA | |
| | | | GGCTCTCCGACTTCCACTGAGGAAGGTACTTCT | |
| | | | ACTGAACCTTCCGAAGGCAGCGCACCAGGTACC | |
| | | | TCTACTGAACCTTCTGAGGGCAGCGCTCCAGGT | |
| | | | AGCGAACCTGCAACCTCTGGCTCTGAAACCCCA | |
| | | | GGTACCTCTGAAAGCGCTACTCCTGAATCTGGC | |
| | | | CCAGGTACTTCTACTGAACCGTCCGAGGGCAGC | |
| | | | GCACCAGGTTTTCCGACTATTCCGCTGTCTCGTC | |
| | | | TGTTTGATAATGCTATGCTGCGTGCGCACCGTC | |
| | | | TGCACCAGCTGGCCTTTGATACTTACCAGGAAT | |
| | | | TTGAAGAAGCcTACATTCCTAAAGAGCAGAAGT | |
| | | | ACTCTTTCCTGCAAAACCCACAGACTTCTCTCTG | |
| | | | CTTCAGCGAATCTATTCCGACGCCTTCCAATCG | |
| | | | CGAGGAAACTCAGCAAAAGTCCAATCTGGAAC | |
| | | | TACTCCGCATTTCTCTGCTTCTGATTCAGAGCTG | |
| | | | GCTAGAACCAGTGCAATTTCTGCGTTCCGTCTT | |
| | | | CGCCAATAGCCTAGTTTATGGCGCATCCGACAG | |
| | | | CAACGTATACGATCTCCTGAAAGATCTCGAGGA | |
| | | | AGGCATTCAGACCCTGATGGGTCGTCTCGAGGA | |
| | | | TGGCTCTCCGCGTACTGGTCAGATCTTCAAGCA | |
| | | | GACTTACTCTAAATTTGATACTAACAGCCACAA | |
| | | | TGACGATGCGCTTCTAAAAAACTATGGTCTGCT | |
| | | | GTATTGTTTTCGTAAAGATATGGACAAAGTTGA | |
| | | | AACCTTCCTGCGTATTGTTCAGTGTCGTTCCGTT | |
| | | | GAGGGCAGCTGTGGTTTCTAA | |
| AE912- | AEPAGSPTSTEEGTPGS | 9 | ATGGCTGAACCTGCTGGCTCTCCAACCTCCACT | 10 |
| hGH- | GTASSSPGSSTPSGATG | | GAGGAAGGTACCCCGGGTAGCGGTACTGCTTCT | |
| AE144 | SPGASPGTSSTGSPGSP | | TCCTCTCCAGGTAGCTCTACCCCTTCTGGTGCAA | |
| | AGSPTSTEEGTSESATP | | CCGGCTCTCCAGGTGCTTCTCCGGGCACCAGCT | |
| | ESGPGTSTEPSEGSAPG | | CTACCGGTTCTCCAGGTAGCCCGGCTGGCTCTC | |
| | SPAGSPTSTEEGTSTEPS | | CTACCTCTACTGAGGAAGGTACTTCTGAAAGCG | |
| | EGSAPGTSTEPSEGSAP | | CTACTCCTGAGTCTGGTCCAGGTACCTCTACTG | |
| | GTSESATPESGPGSEPA | | AACCGTCCGAAGGTAGCGCTCCAGGTAGCCCA | |
| | TSGSETPGSEPATSGSET | | GCAGGCTCTCCGACTTCCACTGAGGAAGGTACT | |
| | PGSPAGSPTSTEEGTSES | | TCTACTGAACCTTCCGAAGGCAGCGCACCAGGT | |
| | ATPESGPGTSTEPSEGS | | ACCTCTACTGAACCTTCTGAGGGCAGCGCTCCA | |
| | APGTSTEPSEGSAPGSP | | GGTACTTCTGAAAGCGCTACCCCGGAATCTGGC | |
| | AGSPTSTEEGTSTEPSE | | CCAGGTAGCGAACCGGCTACTTCTGGTTCTGAA | |
| | GSAPGTSTEPSEGSAPG | | ACCCCAGGTAGCGAACCGGCTACCTCCGGTTCT | |
| | TSESATPESGPGTSTEPS | | GAAACTCCAGGTAGCCCGGCAGGCTCTCCGACC | |
| | EGSAPGTSESATPESGP | | TCTACTGAGGAAGGTACTTCTGAAAGCGCAACC | |
| | GSEPATSGSETPGTSTEP | | CCGGAGTCCGGCCCAGGTACCTCTACCGAACCG | |
| | SEGSAPGTSTEPSEGSA | | TCTGAGGGCAGCGCACCAGGTACTTCTACCGAA | |
| | PGTSESATPESGPGTSES | | CCGTCCGAGGGTAGCGCACCAGGTAGCCCAGC | |
| | ATPESGPGSPAGSPTST | | AGGTTCTCCTACCTCCACCGAGGAAGGTACTTC | |
| | EEGTSESATPESGPGSEP | | TACCGAACCGTCCGAGGGTAGCGCACCAGGTA | |
| | ATSGSETPGTSESATPES | | CCTCTACTGAACCTTCTGAGGGCAGCGCTCCAG | |
| | GPGTSTEPSEGSAPGTS | | GTACTTCTGAAAGCGCTACCCCGGAGTCCGGTC | |
| | TEPSEGSAPGTSTEPSEG | | CAGGTACTTCTACTGAACCGTCCGAAGGTAGCG | |
| | SAPGTSTEPSEGSAPGT | | CACCAGGTACTTCTGAAAGCGCAACCCCTGAAT | |
| | STEPSEGSAPGTSTEPSE | | CCGGTCCAGGTAGCGAACCGGCTACTTCTGGCT | |
| | GSAPGSPAGSPTSTEEG | | CTGAGACTCCAGGTACTTCTACCGAACCGTCCG | |
| | TSTEPSEGSAPGTSESAT | | AAGGTAGCGCACCAGGTACTTCTACTGAACCGT | |
| | PESGPGSEPATSGSETP | | CTGAAGGTAGCGCACCAGGTACTTCTGAAAGCG | |
| | GTSESATPESGPGSEPA | | CAACCCCGGAATCCGGCCCAGGTACCTCTGAAA | |
| | TSGSETPGTSESATPESG | | GCGCAACCCCGGAGTCCGGCCCAGGTAGCCCTG | |
| | PGTSTEPSEGSAPGTSES | | CTGGCTCTCCAACCTCCACCGAAGAAGGTACCT | |
| | ATPESGPGSPAGSPTST | | CTGAAAGCGCAACCCCTGAATCCGGCCCAGGTA | |
| | EEGSPAGSPTSTEEGSP | | GCGAACCGGCAACCTCCGGTTCTGAAACCCCAG | |
| | AGSPTSTEEGTSESATP | | GTACCTCTGAAAGCGCTACTCCGGAGTCTGGCC | |
| | ESGPGTSTEPSEGSAPG | | CAGGTACCTCTACTGAACCGTCTGAGGGTAGCG | |
| | TSESATPESGPGSEPATS | | CTCCAGGTACTTCTACTGAACCGTCCGAAGGTA | |
| | GSETPGTSESATPESGP | | GCGCACCAGGTACTTCTACCGAACCGTCCGAAG | |
| | GSEPATSGSETPGTSES | | GCAGCGCTCCAGGTACCTCTACTGAACCTTCCG | |
| | ATPESGPGTSTEPSEGS | | AGGGCAGCGCTCCAGGTACCTCTACCGAACCTT | |
| | APGSPAGSPTSTEEGTS | | CTGAAGGTAGCGCACCAGGTACTTCTACCGAAC | |
| | ESATPESGPGSEPATSG | | CGTCCGAGGGTAGCGCACCAGGTAGCCCAGCA | |
| | SETPGTSESATPESGPGS | | GGTTCTCCTACCTCCACCGAGGAAGGTACTTCT | |
| | PAGSPTSTEEGSPAGSP | | ACCGAACCGTCCGAGGGTAGCGCACCAGGTAC | |
| | TSTEEGTSTEPSEGSAP | | CTCTGAAAGCGCAACTCCTGAGTCTGGCCCAGG | |
| | GTSESATPESGPGTSES | | TAGCGAACCTGCTACCTCCGGCTCTGAGACTCC | |
| | ATPESGPGTSESATPES | | AGGTACCTCTGAAAGCGCAACCCCGGAATCTGG | |
| | GPGSEPATSGSETPGSE | | TCCAGGTAGCGAACCTGCAACCTCTGGCTCTGA | |
| | PATSGSETPGSPAGSPTS | | AACCCCAGGTACCTCTGAAAGCGCTACTCCTGA | |
| | TEEGTSTEPSEGSAPGT | | ATCTGGCCCAGGTACTTCTACTGAACCGTCCGA | |
| | STEPSEGSAPGSEPATS | | GGGCAGCGCACCAGGTACTTCTGAAAGCGCTAC | |
| | GSETPGTSESATPESGP | | TCCTGAGTCCGGCCCAGGTAGCCCGGCTGGCTC | |
| | GTSTEPSEGSAPGFPTIP | | TCCGACTTCCACCGAGGAAGGTAGCCCGGCTGG | |
| | LSRLFDNAMLRAHRLH | | CTCTCCAACTTCTACTGAAGAAGGTAGCCCGGC | |
| | QLAFDTYQEFEEAYIPK | | AGGCTCTCCGACCTCTACTGAGGAAGGTACTTC | |
| | EQKYSFLQNPQTSLCFS | | TGAAAGCGCAACCCCGGAGTCCGGCCCAGGTA | |
| | ESIPTPSNREETQQKSNL | | CCTCTACCGAACCGTCTGAGGGCAGCGCACCAG | |
| | ELLRISLLLIQSWLEPVQ | | GTACCTCTGAAAGCGCAACTCCTGAGTCTGGCC | |
| | FLRSVFANSLVYGASDS | | CAGGTAGCGAACCTGCTACCTCCGGCTCTGAGA | |
| | NVYDLLKDLEEGIQTL | | CTCCAGGTACCTCTGAAAGCGCAACCCCGGAAT | |
| | MGRLEDGSPRTGQIFK | | CTGGTCCAGGTAGCGAACCTGCAACCTCTGGCT | |
| | QTYSKFDTNSHNDDAL | | CTGAAACCCCAGGTACCTCTGAAAGCGCTACTC | |
| | LKNYGLLYCFRKDMD | | CTGAATCTGGCCCAGGTACTTCTACTGAACCGT | |
| | KVETFLRIVQCRSVEGS | | CCGAGGGCAGCGCACCAGGTAGCCCTGCTGGCT | |
| | CGFGGTSESATPESGPG | | CTCCAACCTCCACCGAAGAAGGTACCTCTGAAA | |
| | TSTEPSEGSAPGTSTEPS | | GCGCAACCCCTGAATCCGGCCCAGGTAGCGAA | |
| | EGSAPGTSESATPESGP | | CCGGCAACCTCCGGTTCTGAAACCCCAGGTACT | |
| | GTSTEPSEGSAPGTSTEP | | TCTGAAAGCGCTACTCCTGAGTCCGGCCCAGGT | |
| | SEGSAPGTSESATPESG | | AGCCCGGCTGGCTCTCCGACTTCCACCGAGGAA | |
| | PGTSTEPSEGSAPGTSTE | | GGTAGCCCGGCTGGCTCTCCAACTTCTACTGAA | |
| | PSEGSAPGTSTEPSEGS | | GAAGGTACTTCTACCGAACCTTCCGAGGGCAGC | |
| | APGSPAGSPTSTEEGTS | | GCACCAGGTACTTCTGAAAGCGCTACCCCTGAG | |
| | TEPSEGSAPG | | TCCGGCCCAGGTACTTCTGAAAGCGCTACTCCT | |
| | | | GAATCCGGTCCAGGTACTTCTGAAAGCGCTACC | |
| | | | CCGGAATCTGGCCCAGGTAGCGAACCGGCTACT | |
| | | | TCTGGTTCTGAAACCCCAGGTAGCGAACCGGCT | |
| | | | ACCTCCGGTTCTGAAACTCCAGGTAGCCCAGCA | |
| | | | GGCTCTCCGACTTCCACTGAGGAAGGTACTTCT | |
| | | | ACTGAACCTTCCGAAGGCAGCGCACCAGGTACC | |
| | | | TCTACTGAACCTTCTGAGGGCAGCGCTCCAGGT | |
| | | | AGCGAACCTGCAACCTCTGGCTCTGAAACCCCA | |
| | | | GGTACCTCTGAAAGCGCTACTCCTGAATCTGGC | |
| | | | CCAGGTACTTCTACTGAACCGTCCGAGGGCAGC | |
| | | | GCACCAGGTTTTCCGACTATTCCGCTGTCTCGTC | |
| | | | TGTTTGATAATGCTATGCTGCGTGCGCACCGTC | |
| | | | TGCACCAGCTGGCCTTTGATACTTACCAGGAAT | |
| | | | TTGAAGAAGCcTACATTCCTAAAGAGCAGAAGT | |
| | | | ACTCTTTCCTGCAAAACCCACAGACTTCTCTCTG | |
| | | | CTTCAGCGAATCTATTCCGACGCCTTCCAATCG | |
| | | | CGAGGAAACTCAGCAAAAGTCCAATCTGGAAC | |
| | | | TACTCCGCATTTCTCTGCTTCTGATTCAGAGCTG | |
| | | | GCTAGAACCAGTGCAATTTCTGCGTTCCGTCTT | |
| | | | CGCCAATAGCCTAGTTTATGGCGCATCCGACAG | |
| | | | CAACGTATACGATCTCCTGAAAGATCTCGAGGA | |
| | | | AGGCATTCAGACCCTGATGGGTCGTCTCGAGGA | |
| | | | TGGCTCTCCGCGTACTGGTCAGATCTTCAAGCA | |
| | | | GACTTACTCTAAATTTGATACTAACAGCCACAA | |
| | | | TGACGATGCGCTTCTAAAAAACTATGGTCTGCT | |
| | | | GTATTGTTTTCGTAAAGATATGGACAAAGTTGA | |
| | | | AACCTTCCTGCGTATTGTTCAGTGTCGTTCCGTT | |
| | | | GAGGGCAGCTGTGGTTTCTAAGGTGGTAGCGAA | |
| | | | CCGGCAACTTCCGGCTCTGAAACCCCAGGTACT | |
| | | | TCTGAAAGCGCTACTCCTGAGTCTGGCCCAGGT | |
| | | | AGCGAACCTGCTACCTCTGGCTCTGAAACCCCA | |
| | | | GGTAGCCCGGCAGGCTCTCCGACTTCCACCGAG | |
| | | | GAAGGTACCTCTACTGAACCTTCTGAGGGTAGC | |
| | | | GCTCCAGGTAGCGAACCGGCAACCTCTGGCTCT | |
| | | | GAAACCCCAGGTAGCGAACCTGCTACCTCCGGC | |
| | | | TCTGAAACTCCAGGTAGCGAACCGGCTACTTCC | |
| | | | GGTTCTGAAACTCCAGGTACCTCTACCGAACCT | |
| | | | TCCGAAGGCAGCGCACCAGGTACTTCTGAAAGC | |
| | | | GCAACCCCTGAATCCGGTCCAGGTAGCGAACCG | |
| | | | GCTACTTCTGGCTCTGAGACTCCAGGTACTTCT | |
| | | | ACCGAACCGTCCGAAGGTAGCGCACCA | |
| AE912- | AEPAGSPTSTEEGTPGS | 11 | ATGGCTGAACCTGCTGGCTCTCCAACCTCCACT | 12 |
| hGH- | GTASSSPGSSTPSGATG | | GAGGAAGGTACCCCGGGTAGCGGTACTGCTTCT | |
| AE288 | SPGASPGTSSTGSPGSP | | TCCTCTCCAGGTAGCTCTACCCCTTCTGGTGCAA | |
| | AGSPTSTEEGTSESATP | | CCGGCTCTCCAGGTGCTTCTCCGGGCACCAGCT | |
| | ESGPGTSTEPSEGSAPG | | CTACCGGTTCTCCAGGTAGCCCGGCTGGCTCTC | |
| | SPAGSPTSTEEGTSTEPS | | CTACCTCTACTGAGGAAGGTACTTCTGAAAGCG | |
| | EGSAPGTSTEPSEGSAP | | CTACTCCTGAGTCTGGTCCAGGTACCTCTACTG | |
| | GTSESATPESGPGSEPA | | AACCGTCCGAAGGTAGCGCTCCAGGTAGCCCA | |
| | TSGSETPGSEPATSGSET | | GCAGGCTCTCCGACTTCCACTGAGGAAGGTACT | |
| | PGSPAGSPTSTEEGTSES | | TCTACTGAACCTTCCGAAGGCAGCGCACCAGGT | |
| | ATPESGPGTSTEPSEGS | | ACCTCTACTGAACCTTCTGAGGGCAGCGCTCCA | |
| | APGTSTEPSEGSAPGSP | | GGTACTTCTGAAAGCGCTACCCCGGAATCTGGC | |
| | AGSPTSTEEGTSTEPSE | | CCAGGTAGCGAACCGGCTACTTCTGGTTCTGAA | |
| | GSAPGTSTEPSEGSAPG | | ACCCCAGGTAGCGAACCGGCTACCTCCGGTTCT | |
| | TSESATPESGPGTSTEPS | | GAAACTCCAGGTAGCCCGGCAGGCTCTCCGACC | |
| | EGSAPGTSESATPESGP | | TCTACTGAGGAAGGTACTTCTGAAAGCGCAACC | |
| | GSEPATSGSETPGTSTEP | | CCGGAGTCCGGCCCAGGTACCTCTACCGAACCG | |
| | SEGSAPGTSTEPSEGSA | | TCTGAGGGCAGCGCACCAGGTACTTCTACCGAA | |
| | PGTSESATPESGPGTSES | | CCGTCCGAGGGTAGCGCACCAGGTAGCCCAGC | |
| | ATPESGPGSPAGSPTST | | AGGTTCTCCTACCTCCACCGAGGAAGGTACTTC | |
| | EEGTSESATPESGPGSEP | | TACCGAACCGTCCGAGGGTAGCGCACCAGGTA | |
| | ATSGSETPGTSESATPES | | CCTCTACTGAACCTTCTGAGGGCAGCGCTCCAG | |
| | GPGTSTEPSEGSAPGTS | | GTACTTCTGAAAGCGCTACCCCGGAGTCCGGTC | |
| | TEPSEGSAPGTSTEPSEG | | CAGGTACTTCTACTGAACCGTCCGAAGGTAGCG | |
| | SAPGTSTEPSEGSAPGT | | CACCAGGTACTTCTGAAAGCGCAACCCCTGAAT | |
| | STEPSEGSAPGTSTEPSE | | CCGGTCCAGGTAGCGAACCGGCTACTTCTGGCT | |
| | GSAPGSPAGSPTSTEEG | | CTGAGACTCCAGGTACTTCTACCGAACCGTCCG | |
| | TSTEPSEGSAPGTSESAT | | AAGGTAGCGCACCAGGTACTTCTACTGAACCGT | |
| | PESGPGSEPATSGSETP | | CTGAAGGTAGCGCACCAGGTACTTCTGAAAGCG | |
| | GTSESATPESGPGSEPA | | CAACCCCGGAATCCGGCCCAGGTACCTCTGAAA | |
| | TSGSETPGTSESATPESG | | GCGCAACCCCGGAGTCCGGCCCAGGTAGCCCTG | |
| | PGTSTEPSEGSAPGTSES | | CTGGCTCTCCAACCTCCACCGAAGAAGGTACCT | |
| | ATPESGPGSPAGSPTST | | CTGAAAGCGCAACCCCTGAATCCGGCCCAGGTA | |
| | EEGSPAGSPTSTEEGSP | | GCGAACCGGCAACCTCCGGTTCTGAAACCCCAG | |
| | AGSPTSTEEGTSESATP | | GTACCTCTGAAAGCGCTACTCCGGAGTCTGGCC | |
| | ESGPGTSTEPSEGSAPG | | CAGGTACCTCTACTGAACCGTCTGAGGGTAGCG | |
| | TSESATPESGPGSEPATS | | CTCCAGGTACTTCTACTGAACCGTCCGAAGGTA | |
| | GSETPGTSESATPESGP | | GCGCACCAGGTACTTCTACCGAACCGTCCGAAG | |
| | GSEPATSGSETPGTSES | | GCAGCGCTCCAGGTACCTCTACTGAACCTTCCG | |
| | ATPESGPGTSTEPSEGS | | AGGGCAGCGCTCCAGGTACCTCTACCGAACCTT | |
| | APGSPAGSPTSTEEGTS | | CTGAAGGTAGCGCACCAGGTACTTCTACCGAAC | |
| | ESATPESGPGSEPATSG | | CGTCCGAGGGTAGCGCACCAGGTAGCCCAGCA | |
| | SETPGTSESATPESGPGS | | GGTTCTCCTACCTCCACCGAGGAAGGTACTTCT | |
| | PAGSPTSTEEGSPAGSP | | ACCGAACCGTCCGAGGGTAGCGCACCAGGTAC | |
| | TSTEEGTSTEPSEGSAP | | CTCTGAAAGCGCAACTCCTGAGTCTGGCCCAGG | |
| | GTSESATPESGPGTSES | | TAGCGAACCTGCTACCTCCGGCTCTGAGACTCC | |
| | ATPESGPGTSESATPES | | AGGTACCTCTGAAAGCGCAACCCCGGAATCTGG | |
| | GPGSEPATSGSETPGSE | | TCCAGGTAGCGAACCTGCAACCTCTGGCTCTGA | |
| | PATSGSETPGSPAGSPTS | | AACCCCAGGTACCTCTGAAAGCGCTACTCCTGA | |
| | TEEGTSTEPSEGSAPGT | | ATCTGGCCCAGGTACTTCTACTGAACCGTCCGA | |
| | STEPSEGSAPGSEPATS | | GGGCAGCGCACCAGGTACTTCTGAAAGCGCTAC | |
| | GSETPGTSESATPESGP | | TCCTGAGTCCGGCCCAGGTAGCCCGGCTGGCTC | |
| | GTSTEPSEGSAPGFPTIP | | TCCGACTTCCACCGAGGAAGGTAGCCCGGCTGG | |
| | LSRLFDNAMLRAHRLH | | CTCTCCAACTTCTACTGAAGAAGGTAGCCCGGC | |
| | QLAFDTYQEFEEAYIPK | | AGGCTCTCCGACCTCTACTGAGGAAGGTACTTC | |
| | EQKYSFLQNPQTSLCFS | | TGAAAGCGCAACCCCGGAGTCCGGCCCAGGTA | |
| | ESIPTPSNREETQQKSNL | | CCTCTACCGAACCGTCTGAGGGCAGCGCACCAG | |
| | ELLRISLLLIQSWLEPVQ | | GTACCTCTGAAAGCGCAACTCCTGAGTCTGGCC | |
| | FLRSVFANSLVYGASDS | | CAGGTAGCGAACCTGCTACCTCCGGCTCTGAGA | |
| | NVYDLLKDLEEGIQTL | | CTCCAGGTACCTCTGAAAGCGCAACCCCGGAAT | |
| | MGRLEDGSPRTGQIFK | | CTGGTCCAGGTAGCGAACCTGCAACCTCTGGCT | |
| | QTYSKFDTNSHNDDAL | | CTGAAACCCCAGGTACCTCTGAAAGCGCTACTC | |
| | LKNYGLLYCFRKDMD | | CTGAATCTGGCCCAGGTACTTCTACTGAACCGT | |
| | KVETFLRIVQCRSVEGS | | CCGAGGGCAGCGCACCAGGTAGCCCTGCTGGCT | |
| | CGFGGTSESATPESGPG | | CTCCAACCTCCACCGAAGAAGGTACCTCTGAAA | |
| | SEPATSGSETPGTSESAT | | GCGCAACCCCTGAATCCGGCCCAGGTAGCGAA | |
| | PESGPGSEPATSGSETP | | CCGGCAACCTCCGGTTCTGAAACCCCAGGTACT | |
| | GTSESATPESGPGTSTEP | | TCTGAAAGCGCTACTCCTGAGTCCGGCCCAGGT | |
| | SEGSAPGSPAGSPTSTE | | AGCCCGGCTGGCTCTCCGACTTCCACCGAGGAA | |
| | EGTSESATPESGPGSEP | | GGTAGCCCGGCTGGCTCTCCAACTTCTACTGAA | |
| | ATSGSETPGTSESATPES | | GAAGGTACTTCTACCGAACCTTCCGAGGGCAGC | |
| | GPGSPAGSPTSTEEGSP | | GCACCAGGTACTTCTGAAAGCGCTACCCCTGAG | |
| | AGSPTSTEEGTSTEPSE | | TCCGGCCCAGGTACTTCTGAAAGCGCTACTCCT | |
| | GSAPGTSESATPESGPG | | GAATCCGGTCCAGGTACTTCTGAAAGCGCTACC | |
| | TSESATPESGPGTSESAT | | CCGGAATCTGGCCCAGGTAGCGAACCGGCTACT | |
| | PESGPGSEPATSGSETP | | TCTGGTTCTGAAACCCCAGGTAGCGAACCGGCT | |
| | GSEPATSGSETPGSPAG | | ACCTCCGGTTCTGAAACTCCAGGTAGCCCAGCA | |
| | SPTSTEEGTSTEPSEGSA | | GGCTCTCCGACTTCCACTGAGGAAGGTACTTCT | |
| | PGTSTEPSEGSAPGSEP | | ACTGAACCTTCCGAAGGCAGCGCACCAGGTACC | |
| | ATSGSETPGTSESATPES | | TCTACTGAACCTTCTGAGGGCAGCGCTCCAGGT | |
| | GPGTSTEPSEGSAPG | | AGCGAACCTGCAACCTCTGGCTCTGAAACCCCA | |
| | | | GGTACCTCTGAAAGCGCTACTCCTGAATCTGGC | |
| | | | CCAGGTACTTCTACTGAACCGTCCGAGGGCAGC | |
| | | | GCACCAGGTTTTCCGACTATTCCGCTGTCTCGTC | |
| | | | TGTTTGATAATGCTATGCTGCGTGCGCACCGTC | |
| | | | TGCACCAGCTGGCCTTTGATACTTACCAGGAAT | |
| | | | TTGAAGAAGCcTACATTCCTAAAGAGCAGAAGT | |
| | | | ACTCTTTCCTGCAAAACCCACAGACTTCTCTCTG | |
| | | | CTTCAGCGAATCTATTCCGACGCCTTCCAATCG | |
| | | | CGAGGAAACTCAGCAAAAGTCCAATCTGGAAC | |
| | | | TACTCCGCATTTCTCTGCTTCTGATTCAGAGCTG | |
| | | | GCTAGAACCAGTGCAATTTCTGCGTTCCGTCTT | |
| | | | CGCCAATAGCCTAGTTTATGGCGCATCCGACAG | |
| | | | CAACGTATACGATCTCCTGAAAGATCTCGAGGA | |
| | | | AGGCATTCAGACCCTGATGGGTCGTCTCGAGGA | |
| | | | TGGCTCTCCGCGTACTGGTCAGATCTTCAAGCA | |
| | | | GACTTACTCTAAATTTGATACTAACAGCCACAA | |
| | | | TGACGATGCGCTTCTAAAAAACTATGGTCTGCT | |
| | | | GTATTGTTTTCGTAAAGATATGGACAAAGTTGA | |
| | | | AACCTTCCTGCGTATTGTTCAGTGTCGTTCCGTT | |
| | | | GAGGGCAGCTGTGGTTTCTAAGGTGGTACCTCT | |
| | | | GAAAGCGCAACTCCTGAGTCTGGCCCAGGTAGC | |
| | | | GAACCTGCTACCTCCGGCTCTGAGACTCCAGGT | |
| | | | ACCTCTGAAAGCGCAACCCCGGAATCTGGTCCA | |
| | | | GGTAGCGAACCTGCAACCTCTGGCTCTGAAACC | |
| | | | CCAGGTACCTCTGAAAGCGCTACTCCTGAATCT | |
| | | | GGCCCAGGTACTTCTACTGAACCGTCCGAGGGC | |
| | | | AGCGCACCAGGTAGCCCTGCTGGCTCTCCAACC | |
| | | | TCCACCGAAGAAGGTACCTCTGAAAGCGCAAC | |
| | | | CCCTGAATCCGGCCCAGGTAGCGAACCGGCAA | |
| | | | CCTCCGGTTCTGAAACCCCAGGTACTTCTGAAA | |
| | | | GCGCTACTCCTGAGTCCGGCCCAGGTAGCCCGG | |
| | | | CTGGCTCTCCGACTTCCACCGAGGAAGGTAGCC | |
| | | | CGGCTGGCTCTCCAACTTCTACTGAAGAAGGTA | |
| | | | CTTCTACCGAACCTTCCGAGGGCAGCGCACCAG | |
| | | | GTACTTCTGAAAGCGCTACCCCTGAGTCCGGCC | |
| | | | CAGGTACTTCTGAAAGCGCTACTCCTGAATCCG | |
| | | | GTCCAGGTACTTCTGAAAGCGCTACCCCGGAAT | |
| | | | CTGGCCCAGGTAGCGAACCGGCTACTTCTGGTT | |
| | | | CTGAAACCCCAGGTAGCGAACCGGCTACCTCCG | |
| | | | GTTCTGAAACTCCAGGTAGCCCAGCAGGCTCTC | |
| | | | CGACTTCCACTGAGGAAGGTACTTCTACTGAAC | |
| | | | CTTCCGAAGGCAGCGCACCAGGTACCTCTACTG | |
| | | | AACCTTCTGAGGGCAGCGCTCCAGGTAGCGAAC | |
| | | | CTGCAACCTCTGGCTCTGAAACCCCAGGTACCT | |
| | | | CTGAAAGCGCTACTCCTGAATCTGGCCCAGGTA | |
| | | | CTTCTACTGAACCGTCCGAGGGCAGCGCACCA | |
| AM875- | GTSTEPSEGSAPGSEPA | 13 | GGTACTTCTACTGAACCGTCTGAAGGCAGCGCA | 14 |
| hGH | TSGSETPGSPAGSPTSTE | | CCAGGTAGCGAACCGGCTACTTCCGGTTCTGAA | |
| | EGSTSSTAESPGPGTSTP | | ACCCCAGGTAGCCCAGCAGGTTCTCCAACTTCT | |
| | ESGSASPGSTSESPSGTA | | ACTGAAGAAGGTTCTACCAGCTCTACCGCAGAA | |
| | PGSTSESPSGTAPGTSTP | | TCTCCTGGTCCAGGTACCTCTACTCCGGAAAGC | |
| | ESGSASPGTSTPESGSAS | | GGCTCTGCATCTCCAGGTTCTACTAGCGAATCT | |
| | PGSEPATSGSETPGTSES | | CCTTCTGGCACTGCACCAGGTTCTACTAGCGAA | |
| | ATPESGPGSPAGSPTST | | TCCCCGTCTGGTACTGCTCCAGGTACTTCTACTC | |
| | EEGTSTEPSEGSAPGTS | | CTGAAAGCGGTTCCGCTTCTCCAGGTACCTCTA | |
| | ESATPESGPGTSTEPSEG | | CTCCGGAAAGCGGTTCTGCATCTCCAGGTAGCG | |
| | SAPGTSTEPSEGSAPGSP | | AACCGGCAACCTCCGGCTCTGAAACCCCAGGTA | |
| | AGSPTSTEEGTSTEPSE | | CCTCTGAAAGCGCTACTCCTGAATCCGGCCCAG | |
| | GSAPGTSTEPSEGSAPG | | GTAGCCCGGCAGGTTCTCCGACTTCCACTGAGG | |
| | TSESATPESGPGTSESAT | | AAGGTACCTCTACTGAACCTTCTGAGGGCAGCG | |
| | PESGPGTSTEPSEGSAP | | CTCCAGGTACTTCTGAAAGCGCTACCCCGGAGT | |
| | GTSTEPSEGSAPGTSES | | CCGGTCCAGGTACTTCTACTGAACCGTCCGAAG | |
| | ATPESGPGTSTEPSEGS | | GTAGCGCACCAGGTACTTCTACCGAACCGTCCG | |
| | APGSEPATSGSETPGSP | | AGGGTAGCGCACCAGGTAGCCCAGCAGGTTCTC | |
| | AGSPTSTEEGSSTPSGA | | CTACCTCCACCGAGGAAGGTACTTCTACCGAAC | |
| | TGSPGTPGSGTASSSPG | | CGTCCGAGGGTAGCGCACCAGGTACTTCTACCG | |
| | SSTPSGATGSPGTSTEPS | | AACCTTCCGAGGGCAGCGCACCAGGTACTTCTG | |
| | EGSAPGTSTEPSEGSAP | | AAAGCGCTACCCCTGAGTCCGGCCCAGGTACTT | |
| | GSEPATSGSETPGSPAG | | CTGAAAGCGCTACTCCTGAATCCGGTCCAGGTA | |
| | SPTSTEEGSPAGSPTSTE | | CCTCTACTGAACCTTCCGAAGGCAGCGCTCCAG | |
| | EGTSTEPSEGSAPGASA | | GTACCTCTACCGAACCGTCCGAGGGCAGCGCAC | |
| | SGAPSTGGTSESATPES | | CAGGTACTTCTGAAAGCGCAACCCCTGAATCCG | |
| | GPGSPAGSPTSTEEGSP | | GTCCAGGTACTTCTACTGAACCTTCCGAAGGTA | |
| | AGSPTSTEEGSTSSTAES | | GCGCTCCAGGTAGCGAACCTGCTACTTCTGGTT | |
| | PGPGSTSESPSGTAPGTS | | CTGAAACCCCAGGTAGCCCGGCTGGCTCTCCGA | |
| | PSGESSTAPGTPGSGTA | | CCTCCACCGAGGAAGGTAGCTCTACCCCGTCTG | |
| | SSSPGSSTPSGATGSPGS | | GTGCTACTGGTTCTCCAGGTACTCCGGGCAGCG | |
| | SPSASTGTGPGSEPATS | | GTACTGCTTCTTCCTCTCCAGGTAGCTCTACCCC | |
| | GSETPGTSESATPESGP | | TTCTGGTGCTACTGGCTCTCCAGGTACCTCTACC | |
| | GSEPATSGSETPGSTSST | | GAACCGTCCGAGGGTAGCGCACCAGGTACCTCT | |
| | AESPGPGSTSSTAESPGP | | ACTGAACCGTCTGAGGGTAGCGCTCCAGGTAGC | |
| | GTSPSGESSTAPGSEPA | | GAACCGGCAACCTCCGGTTCTGAAACTCCAGGT | |
| | TSGSETPGSEPATSGSET | | AGCCCTGCTGGCTCTCCGACTTCTACTGAGGAA | |
| | PGTSTEPSEGSAPGSTSS | | GGTAGCCCGGCTGGTTCTCCGACTTCTACTGAG | |
| | TAESPGPGTSTPESGSA | | GAAGGTACTTCTACCGAACCTTCCGAAGGTAGC | |
| | SPGSTSESPSGTAPGTST | | GCTCCAGGTGCAAGCGCAAGCGGCGCGCCAAG | |
| | EPSEGSAPGTSTEPSEGS | | CACGGGAGGTACTTCTGAAAGCGCTACTCCTGA | |
| | APGTSTEPSEGSAPGSS | | GTCCGGCCCAGGTAGCCCGGCTGGCTCTCCGAC | |
| | TPSGATGSPGSSPSAST | | TTCCACCGAGGAAGGTAGCCCGGCTGGCTCTCC | |
| | GTGPGASPGTSSTGSPG | | AACTTCTACTGAAGAAGGTTCTACCAGCTCTAC | |
| | SEPATSGSETPGTSESAT | | CGCTGAATCTCCTGGCCCAGGTTCTACTAGCGA | |
| | PESGPGSPAGSPTSTEE | | ATCTCCGTCTGGCACCGCACCAGGTACTTCCCC | |
| | GSSTPSGATGSPGSSPS | | TAGCGGTGAATCTTCTACTGCACCAGGTACCCC | |
| | ASTGTGPGASPGTSSTG | | TGGCAGCGGTACCGCTTCTTCCTCTCCAGGTAG | |
| | SPGTSESATPESGPGTST | | CTCTACCCCGTCTGGTGCTACTGGCTCTCCAGGT | |
| | EPSEGSAPGTSTEPSEGS | | TCTAGCCCGTCTGCATCTACCGGTACCGGCCCA | |
| | APGFPTIPLSRLFDNAM | | GGTAGCGAACCGGCAACCTCCGGCTCTGAAACT | |
| | LRAHRLHQLAFDTYQE | | CCAGGTACTTCTGAAAGCGCTACTCCGGAATCC | |
| | FEEAYIPKEQKYSFLQN | | GGCCCAGGTAGCGAACCGGCTACTTCCGGCTCT | |
| | PQTSLCFSESIPTPSNRE | | GAAACCCCAGGTTCCACCAGCTCTACTGCAGAA | |
| | ETQQKSNLELLRISLLLI | | TCTCCGGGCCCAGGTTCTACTAGCTCTACTGCA | |
| | QSWLEPVQFLRSVFAN | | GAATCTCCGGGTCCAGGTACTTCTCCTAGCGGC | |
| | SLVYGASDSNVYDLLK | | GAATCTTCTACCGCTCCAGGTAGCGAACCGGCA | |
| | DLEEGIQTLMGRLEDGS | | ACCTCTGGCTCTGAAACTCCAGGTAGCGAACCT | |
| | PRTGQIFKQTYSKFDTN | | GCAACCTCCGGCTCTGAAACCCCAGGTACTTCT | |
| | SHNDDALLKNYGLLYC | | ACTGAACCTTCTGAGGGCAGCGCACCAGGTTCT | |
| | FRKDMDKVETFLRIVQ | | ACCAGCTCTACCGCAGAATCTCCTGGTCCAGGT | |
| | CRSVEGSCGF | | ACCTCTACTCCGGAAAGCGGCTCTGCATCTCCA | |
| | | | GGTTCTACTAGCGAATCTCCTTCTGGCACTGCA | |
| | | | CCAGGTACTTCTACCGAACCGTCCGAAGGCAGC | |
| | | | GCTCCAGGTACCTCTACTGAACCTTCCGAGGGC | |
| | | | AGCGCTCCAGGTACCTCTACCGAACCTTCTGAA | |
| | | | GGTAGCGCACCAGGTAGCTCTACTCCGTCTGGT | |
| | | | GCAACCGGCTCCCCAGGTTCTAGCCCGTCTGCT | |
| | | | TCCACTGGTACTGGCCCAGGTGCTTCCCCGGGC | |
| | | | ACCAGCTCTACTGGTTCTCCAGGTAGCGAACCT | |
| | | | GCTACCTCCGGTTCTGAAACCCCAGGTACCTCT | |
| | | | GAAAGCGCAACTCCGGAGTCTGGTCCAGGTAG | |
| | | | CCCTGCAGGTTCTCCTACCTCCACTGAGGAAGG | |
| | | | TAGCTCTACTCCGTCTGGTGCAACCGGCTCCCC | |
| | | | AGGTTCTAGCCCGTCTGCTTCCACTGGTACTGG | |
| | | | CCCAGGTGCTTCCCCGGGCACCAGCTCTACTGG | |
| | | | TTCTCCAGGTACCTCTGAAAGCGCTACTCCGGA | |
| | | | GTCTGGCCCAGGTACCTCTACTGAACCGTCTGA | |
| | | | GGGTAGCGCTCCAGGTACTTCTACTGAACCGTC | |
| | | | CGAAGGTAGCGCACCAGGTTTTCCGACTATTCC | |
| | | | GCTGTCTCGTCTGTTTGATAATGCTATGCTGCGT | |
| | | | GCGCACCGTCTGCACCAGCTGGCCTTTGATACT | |
| | | | TACCAGGAATTTGAAGAAGCcTACATTCCTAAA | |
| | | | GAGCAGAAGTACTCTTTCCTGCAAAACCCACAG | |
| | | | ACTTCTCTCTGCTTCAGCGAATCTATTCCGACGC | |
| | | | CTTCCAATCGCGAGGAAACTCAGCAAAAGTCCA | |
| | | | ATCTGGAACTACTCCGCATTTCTCTGCTTCTGAT | |
| | | | TCAGAGCTGGCTAGAACCAGTGCAATTTCTGCG | |
| | | | TTCCGTCTTCGCCAATAGCCTAGTTTATGGCGC | |
| | | | ATCCGACAGCAACGTATACGATCTCCTGAAAGA | |
| | | | TCTCGAGGAAGGCATTCAGACCCTGATGGGTCG | |
| | | | TCTCGAGGATGGCTCTCCGCGTACTGGTCAGAT | |
| | | | CTTCAAGCAGACTTACTCTAAATTTGATACTAA | |
| | | | CAGCCACAATGACGATGCGCTTCTAAAAAACTA | |
| | | | TGGTCTGCTGTATTGTTTTCGTAAAGATATGGA | |
| | | | CAAAGTTGAAACCTTCCTGCGTATTGTTCAGTG | |
| | | | TCGTTCCGTTGAGGGCAGCTGTGGTTTCTAA | |

Further characterization of the exemplary hGH-XTEN fusion proteins provided in Table 1 can be found in the examples (e.g., Examples 27-35) of Schellenberger et al. WO10/144502A2, which is incorporated herein by reference in its entirety.

The present invention contemplates use of hGH-XTEN fusion proteins comprising one of the amino acid sequences shown in FIG. 1, Table 1, or as described in Schellenberger et al. WO10/144502A2 (which is incorporated herein by reference in its entirety). In addition, pharmacologically active variants of any of the hGH-XTEN fusion proteins described and referred to herein are also contemplated.

As described more fully below, the fusion proteins optionally include spacer sequences that further comprise cleavage sequences to release the GH from the fusion protein when acted on by a protease, releasing GH from the XTEN sequence(s).

In one aspect, the invention provides an isolated fusion protein comprising at least a first biologically active growth hormone protein covalently linked to one or more extended recombinant polypeptides (“XTEN”), resulting in a growth hormone-XTEN fusion protein composition (hereinafter “hGH-XTEN”). In one embodiment, the growth hormone is human growth hormone or a sequence variant of hGH. As described more fully below, the fusion proteins optionally include spacer sequences that further comprise cleavage sequences to release the GH from the fusion protein when acted on by a protease.

The term “hGH-XTEN”, as used herein, is meant to encompass fusion polypeptides that comprise a payload region comprising a biologically active GH that mediates one or more biological or therapeutic activities associated with growth hormone and at least one other region comprising at least a first XTEN polypeptide that serves as a carrier. In one embodiment, the invention provides an hGH-XTEN fusion protein comprising the sequence set forth in Table 1.

The GH of the subject compositions, together with their corresponding nucleic acid and amino acid sequences, are well known in the art and descriptions and sequences are available in public databases such as Chemical Abstracts Services Databases (e.g., the CAS Registry), GenBank, The Universal Protein Resource (UniProt) and subscription provided databases such as GenSeq (e.g., Derwent). Polynucleotide sequences may be a wild type polynucleotide sequence encoding a given GH (e.g., either full length or mature), or in some instances the sequence may be a variant of the wild type polynucleotide sequence (e.g., a polynucleotide which encodes the wild type biologically active protein, wherein the DNA sequence of the polynucleotide has been optimized, for example, for expression in a particular species; or a polynucleotide encoding a variant of the wild type protein, such as a site directed mutant or an allelic variant. It is well within the ability of the skilled artisan to use a wild-type or consensus cDNA sequence or a codon-optimized variant of a GH to create fusion protein constructs contemplated by the invention using methods known in the art and/or in conjunction with the guidance and methods provided herein, and described more fully in the Examples of Schellenberger et al. WO10/144502A2 which is incorporated herein by reference in its entirety.

The GH for inclusion in the hGH-XTEN of the invention include any growth hormone or sequence variant of biologic, therapeutic, prophylactic, or diagnostic interest or function, or that is useful for mediating or preventing or ameliorating a disease, disorder or condition associated with growth, growth hormone deficiency or defect when administered to a subject. Of particular interest are hGH-XTEN fusion protein compositions for which an increase in a pharmacokinetic parameter, increased solubility, increased stability, or some other enhanced pharmaceutical or pharmacodynamic property compared to native GH is sought, or for which increasing the terminal half-life would improve efficacy, safety, or result in reduce dosing frequency and/or improve patient compliance. Thus, the hGH-XTEN fusion protein compositions are prepared with various objectives in mind, including improving the therapeutic efficacy of the bioactive GH by, for example, increasing the in vivo exposure or the length that the hGH-XTEN remains within the therapeutic window when administered to a subject, compared to a GH not linked to XTEN.

In one embodiment, the GH incorporated into the subject compositions can be a recombinant polypeptide with a sequence corresponding to a protein found in nature, such as human growth hormone. In one embodiment, the GH is human GH comprising the following amino acid sequence:

(SEQ ID NO:2)
FPTIPLSRLFDNAMLRAHRLHQLAFDTYQEFEEAYIPKEQKYSFLQNPQ
TSLCFSESIPTPSNREETQQKSNLELLRISLLLIQSWLEPVQFLRSVFA
NSLVYGASDSNVYDLLKDLEEGIQTLMGRLEDGSPRTGQIFKQTYSKFD
TNSHNDDALLKNYGLLYCFRKDMDKVETFLRIVQCRSVEGSCGF.

In another embodiment, the GH is a sequence variant, fragment, homolog, or mimetic of a natural sequence that retain at least a portion of the biological activity of the native GH. In non-limiting examples, a GH is a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the protein sequence of SEQ ID NO: 2. In one embodiment, the hGH-XTEN fusion protein comprises a single GH molecule linked to an XTEN (as described more fully below). In another embodiment, the hGH-XTEN fusion protein comprises a single GH molecule linked to a first and a second XTEN, with an N— to C-terminus configuration of XTEN-GH-XTEN, in which the GH is a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the human growth hormone protein sequence (SEQ ID NO: 2), and the first and/or the second XTEN are sequences that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to a sequence selected from Table 3.

In general, the GH fusion partner component of the hGH-XTEN exhibits a binding specificity to a given target or another desired biological characteristic when used in vivo or when utilized in an in vitro assay. For example, the hGH-XTEN is an agonist, having the ability to bind to a transmembrane receptor for growth hormone. In one embodiment, the binding of hGH-XTEN to growth receptor leads to receptor dimerization and lead to at least a portion of the activation of intercellular signal transduction pathway compared to native growth hormone.

In one embodiment, the hGH-XTEN bound to a transmembrane receptor for growth hormone would exhibit at least about 1%, or about 5%, or about 10%, or about 15%, or about 20%, or about 25%, or about 30%, or about 40%, or about 50%, or about 60%, or about 70%, or about 80%, or about 90%, or at least about 95% of the activation of intercellular signal transduction pathway compared to native growth hormone not linked to XTEN.

The subject hGH-XTEN of the present invention exhibits an enhancement of one or more pharmacokinetic or pharmacodynamic parameters, which optionally is enhanced by release of GH from the fusion protein by cleavage of a spacer sequence. The hGH-XTEN with enhanced pharmacokinetic parameters permits less frequent dosing or an enhanced pharmacologic effect, such as but not limited to maintaining the biologically active hGH-XTEN within the therapeutic window between the minimum effective dose or blood concentration (Cmin) and the maximum tolerated dose or blood concentration (Cmax). In addition, the hGH-XTEN with enhanced pharmacodynamic parameters permits lower and/or less frequent dosing or an enhanced pharmacodynamic effect, such as but not limited to a sustained or normalized IGF-I standard deviation score (IGF-I SDS). In such cases, the linking of the GH to a fusion protein comprising a select XTEN sequence(s) can result in an improvement in these properties, making them more useful as therapeutic or preventive agents compared to GH not linked to XTEN.

### IV). XTENDED Recombinant Polypeptides

The present invention concerns an improved therapeutic regimen for GHD therapy. In particular, the invention concerns methods for bolus dose administration of a human growth hormone-XTEN (hGH-XTEN) fusion protein to a patient with GHD. Accordingly, in one aspect, the present invention concerns a method of treating human growth hormone deficiency (GHD) with a hGH-XTEN recombinant polypeptide or fusion protein.

In another aspect, the present invention provides XTEN polypeptide compositions that are useful as a fusion protein partner to which GH is linked, resulting in a hGH-XTEN fusion protein. XTEN are generally extended length polypeptides with non-naturally occurring, substantially non-repetitive sequences that are composed mainly of small hydrophilic amino acids, with the sequence having a low degree or no secondary or tertiary structure under physiologic conditions.

XTENs have utility as a fusion protein partners partner in that they serve as a “carrier”, conferring certain desirable pharmacokinetic, physicochemical and pharmaceutical properties when linked to a GH protein to a create a fusion protein. Such desirable properties include but are not limited to enhanced pharmacokinetic parameters and solubility characteristics the compositions, amongst other properties described herein. Such fusion protein compositions have utility to treat certain growth hormone-related diseases, disorders or conditions, as described herein. As used herein, “XTEN” specifically excludes antibodies or antibody fragments such as single-chain antibodies or Fc fragments of a light chain or a heavy chain.

In some embodiments, XTEN are long polypeptides having greater than about 100 to about 3000 amino acid residues, preferably greater than 400 to about 3000 residues when used as a carrier or cumulatively when more than one XTEN unit is used in a single fusion protein. In other embodiments, when used as a linker between fusion protein components or where an increase in half-life of the fusion protein is not needed but where an increase in solubility or other physico/chemical property for the GH fusion partner component is desired, an XTEN sequence shorter than 100 amino acid residues, such as about 96, or about 84, or about 72, or about 60, or about 48, or about 36 amino acid residues are incorporated into a fusion protein composition with the GH to effect the property.

The selection criteria for the XTEN to be linked to the biologically active proteins used to create the inventive fusion proteins compositions generally relate to attributes of physical/chemical properties and conformational structure of the XTEN that is, in turn, used to confer enhanced pharmaceutical and pharmacokinetic properties to the fusion proteins. The XTEN of the present invention exhibit one or more of the following advantageous properties: conformational flexibility, enhanced aqueous solubility, high degree of protease resistance, low immunogenicity, low binding to mammalian receptors, and increased hydrodynamic (or Stokes) radii; properties that make them particularly useful as fusion protein partners. Non-limiting examples of the properties of the fusion proteins comprising GH that is enhanced by XTEN include increases in the overall solubility and/or metabolic stability, reduced susceptibility to proteolysis, reduced immunogenicity, reduced rate of absorption when administered subcutaneously or intramuscularly, and enhanced pharmacokinetic properties such as longer terminal half-life and increased area under the curve (AUC), slower absorption after subcutaneous or intramuscular injection (compared to GH not linked to XTEN and administered by a similar route) such that the Cmaxis lower, which, in turn, results in reductions in adverse effects of the GH that, collectively, results in an increased period of time that a fusion protein of a hGH-XTEN composition administered to a subject retains therapeutic activity.

A variety of methods and assays are known in the art for determining the physical/chemical properties of proteins such as the compositions comprising the inventive XTEN; properties such as secondary or tertiary structure, solubility, protein aggregation, melting properties, contamination and water content. Such methods include analytical centrifugation, EPR, HPLC-ion exchange, HPLC-size exclusion, HPLC-reverse phase, light scattering, capillary electrophoresis, circular dichroism, differential scanning calorimetry, fluorescence,

HPLC-ion exchange, HPLC-size exclusion, IR, NMR, Raman spectroscopy, refractometry, and UV/Visible spectroscopy. Additional methods are disclosed in Arnau et al, Prot Expr and Purif (2006) 48, 1-13. Application of these methods to the invention would be within the grasp of a person skilled in the art.

Typically, XTEN are designed to behave like denatured peptide sequences under physiological conditions, despite the extended length of the polymer. Denatured describes the state of a peptide in solution that is characterized by a large conformational freedom of the peptide backbone. Most peptides and proteins adopt a denatured conformation in the presence of high concentrations of denaturants or at elevated temperature. Peptides in denatured conformation have, for example, characteristic circular dichroism (CD) spectra and are characterized by a lack of long-range interactions as determined by NMR. “Denatured conformation” and “unstructured conformation” are used synonymously herein. In some embodiments, the invention provides XTEN sequences that, under physiologic conditions, resemble denatured sequences largely devoid in secondary structure. In other cases, the XTEN sequences are substantially devoid of secondary structure under physiologic conditions. “Largely devoid,” as used in this context, means that less than 50% of the XTEN amino acid residues of the XTEN sequence contribute to secondary structure as measured or determined by the means described herein. “Substantially devoid,” as used in this context, means that at least about 60%, or about 70%, or about 80%, or about 90%, or about 95%, or at least about 99% of the XTEN amino acid residues of the XTEN sequence do not contribute to secondary structure, as measured or determined by the methods described herein.

A variety of methods have been established in the art to discern the presence or absence of secondary and tertiary structures in a given polypeptide. In particular, secondary structure can be measured spectrophotometrically, e.g., by circular dichroism spectroscopy in the “far-UV” spectral region (190-250 nm). Secondary structure elements, such as alpha-helix and beta-sheet, each give rise to a characteristic shape and magnitude of CD spectra. Secondary structure can also be predicted for a polypeptide sequence via certain computer programs or algorithms, such as the well-known Chou-Fasman algorithm (Chou, P. Y., et al. (1974) Biochemistry, 13: 222-45) and the Garnier-Osguthorpe-Robson (“GOR”) algorithm (Garnier J, Gibrat J F, Robson B. (1996), GOR method for predicting protein secondary structure from amino acid sequence. Methods Enzymol 266:540-553), as described in US Patent Application Publication No. 20030228309A1. For a given sequence, the algorithms can predict whether there exists some or no secondary structure at all, expressed as the total and/or percentage of residues of the sequence that form, for example, alpha-helices or beta-sheets or the percentage of residues of the sequence predicted to result in random coil formation (which lacks secondary structure).

In some embodiments, the XTEN sequences used in the inventive fusion protein compositions can have an alpha-helix percentage ranging from 0% to less than about 5% as determined by the Chou-Fasman algorithm. In other cases, the XTEN sequences of the fusion protein compositions have a beta-sheet percentage ranging from 0% to less than about 5% as determined by the Chou-Fasman algorithm. In some embodiments, the XTEN sequences of the fusion protein compositions have an alpha-helix percentage ranging from 0% to less than about 5% and a beta-sheet percentage ranging from 0% to less than about 5% as determined by the Chou-Fasman algorithm. In some embodiments, the XTEN sequences of the fusion protein compositions have an alpha-helix percentage less than about 2% and a beta-sheet percentage less than about 2%. In other cases, the XTEN sequences of the fusion protein compositions have a high degree of random coil percentage, as determined by the GOR algorithm. In some embodiments, an XTEN sequence have at least about 80%, more preferably at least about 90%, more preferably at least about 91%, more preferably at least about 92%, more preferably at least about 93%, more preferably at least about 94%, more preferably at least about 95%, more preferably at least about 96%, more preferably at least about 97%, more preferably at least about 98%, and most preferably at least about 99% random coil, as determined by the GOR algorithm.

1. Non-Repetitive Sequences

In some embodiments, XTEN sequences of the compositions are substantially non-repetitive. In general, repetitive amino acid sequences have a tendency to aggregate or form higher order structures, as exemplified by natural repetitive sequences such as collagens and leucine zippers, or form contacts resulting in crystalline or pseudocrystalline structures. In contrast, the low tendency of non-repetitive sequences to aggregate enables the design of long-sequence XTENs with a relatively low frequency of charged amino acids that would be likely to aggregate if the sequences were otherwise repetitive. Typically, the hGH-XTEN fusion proteins comprise XTEN sequences of greater than about 100 to about 3000 amino acid residues, preferably greater than 400 to about 3000 cumulative residues, wherein the sequences are substantially non-repetitive. In one embodiment, the XTEN sequences have greater than about 100 to about 3000 amino acid residues, preferably greater than 400 to about 3000 amino acid residues, in which no three contiguous amino acids in the sequence are identical amino acid types unless the amino acid is serine, in which case no more than three contiguous amino acids are serine residues. In the foregoing embodiment, the XTEN sequence would be substantially non-repetitive.

The degree of repetitiveness of a polypeptide or a gene are measured by computer programs or algorithms or by other means known in the art. Repetitiveness in a polypeptide sequence can, for example, be assessed by determining the number of times shorter sequences of a given length occur within the polypeptide. For example, a polypeptide of 200 amino acid residues has 192 overlapping 9-amino acid sequences (or 9-mer “frames”) and 198 3-mer frames, but the number of unique 9-mer or 3-mer sequences will depend on the amount of repetitiveness within the sequence. A score is generated (hereinafter “subsequence score”) that is reflective of the degree of repetitiveness of the subsequences in the overall polypeptide sequence. In the context of the present invention, “subsequence score” means the sum of occurrences of each unique 3-mer frame across a 200 consecutive amino acid sequence of the polypeptide divided by the absolute number of unique 3-mer subsequences within the 200 amino acid sequence. Examples of such subsequence scores derived from the first 200 amino acids of repetitive and non-repetitive polypeptides are presented in Example 44 of Schellenberger et al. WO10/144502A2, which is incorporated herein by reference in its entirety. In some embodiments, the present invention provides hGH-XTEN each comprising one or more XTEN in which the XTEN have a subsequence score less than 12, more preferably less than 10, more preferably less than 9, more preferably less than 8, more preferably less than 7, more preferably less than 6, and most preferably less than 5. In the embodiments hereinabove described in this paragraph, an XTEN with a subsequence score less than about 10 (i.e., 9, 8, 7, etc.) is “substantially non-repetitive.”

The non-repetitive characteristic of XTEN impart to fusion proteins with GH a greater degree of solubility and less tendency to aggregate compared to polypeptides having repetitive sequences. These properties facilitate the formulation of XTEN-comprising pharmaceutical preparations containing extremely high drug concentrations, in some cases exceeding 100 mg/ml.

Furthermore, the XTEN polypeptide sequences of the embodiments are designed to have a low degree of internal repetitiveness in order to reduce or substantially eliminate immunogenicity when administered to a mammal. Polypeptide sequences composed of short, repeated motifs largely limited to three amino acids, such as glycine, serine and glutamate, may result in relatively high antibody titers when administered to a mammal despite the absence of predicted T-cell epitopes in these sequences. This may be caused by the repetitive nature of polypeptides, as it has been shown that immunogens with repeated epitopes, including protein aggregates, cross-linked immunogens, and repetitive carbohydrates are highly immunogenic and can, for example, result in the cross-linking of B-cell receptors causing B-cell activation. (Johansson, J., et al. (2007) Vaccine, 25:1676-82; Yankai, Z., et al. (2006) Biochem Biophys Res Commun, 345:1365-71; Hsu, C. T., et al. (2000) Cancer Res, 60:3701-5); Bachmann M F, et al. Eur J. Immunol. (1995) 25(12):3445-3451).

2. Exemplary Sequence Motifs

The present invention encompasses XTEN that comprise multiple units of shorter sequences, or motifs, in which the amino acid sequences of the motifs are non-repetitive. In designing XTEN sequences, it was discovered that the non-repetitive criterion may be met despite the use of a “building block” approach using a library of sequence motifs that are multimerized to create the XTEN sequences. Thus, while an XTEN sequence may consist of multiple units of as few as four different types of sequence motifs, because the motifs themselves generally consist of non-repetitive amino acid sequences, the overall XTEN sequence is rendered substantially non-repetitive.

In one embodiment, XTEN have a non-repetitive sequence of greater than about 100 to about 3000 amino acid residues, preferably greater than 400 to about 3000 residues, wherein at least about 80%, or at least about 85%, or at least about 90%, or at least about 95%, or at least about 97%, or about 100% of the XTEN sequence consists of non-overlapping sequence motifs, wherein each of the motifs has about 9 to 36 amino acid residues. In other embodiments, at least about 80%, or at least about 85%, or at least about 90%, or at least about 95%, or at least about 97%, or about 100% of the XTEN sequence consists of non-overlapping sequence motifs wherein each of the motifs has 9 to 14 amino acid residues. In still other embodiments, at least about 80%, or at least about 85%, or at least about 90%, or at least about 95%, or at least about 97%, or about 100% of the XTEN sequence component consists of non-overlapping sequence motifs wherein each of the motifs has 12 amino acid residues. In these embodiments, it is preferred that the sequence motifs be composed mainly of small hydrophilic amino acids, such that the overall sequence has an unstructured, flexible characteristic. Examples of amino acids that are included in XTEN, are, e.g., arginine, lysine, threonine, alanine, asparagine, glutamine, aspartate, glutamate, serine, and glycine. As a result of testing variables such as codon optimization, assembly polynucleotides encoding sequence motifs, expression of protein, charge distribution and solubility of expressed protein, and secondary and tertiary structure, it was discovered that XTEN compositions with enhanced characteristics mainly include glycine (G), alanine (A), serine (S), threonine (T), glutamate (E) and proline (P) residues wherein the sequences are designed to be substantially non-repetitive. In one embodiment, XTEN sequences have predominately four to six types of amino acids selected from glycine (G), alanine (A), serine (S), threonine (T), glutamate (E) or proline (P) that are arranged in a substantially non-repetitive sequence that is greater than about 100 to about 3000 amino acid residues, preferably greater than 400 to about 3000 residues in length. In some embodiments, XTEN have sequences of greater than about 100 to about 3000 amino acid residues, preferably greater than 400 to about 3000 residues, wherein at least about 80% of the sequence consists of non-overlapping sequence motifs wherein each of the motifs has 9 to 36 amino acid residues wherein each of the motifs consists of 4 to 6 types of amino acids selected from glycine (G), alanine (A), serine (S), threonine (T), glutamate (E) and proline (P), and wherein the content of any one amino acid type in the full-length XTEN does not exceed 30%. In other embodiments, at least about 90% of the XTEN sequence consists of non-overlapping sequence motifs wherein each of the motifs has 9 to 36 amino acid residues wherein the motifs consist of 4 to 6 types of amino acids selected from glycine (G), alanine (A), serine (S), threonine (T), glutamate (E) and proline (P), and wherein the content of any one amino acid type in the full-length XTEN does not exceed 30%. In other embodiments, at least about 90% of the XTEN sequence consists of non-overlapping sequence motifs wherein each of the motifs has 12 amino acid residues consisting of 4 to 6 types of amino acids selected from glycine (G), alanine (A), serine (S), threonine (T), glutamate (E) and proline (P), and wherein the content of any one amino acid type in the full-length XTEN does not exceed 30%. In yet other embodiments, at least about 90%, or about 91%, or about 92%, or about 93%, or about 94%, or about 95%, or about 96%, or about 97%, or about 98%, or about 99%, to about 100% of the XTEN sequence consists of non-overlapping sequence motifs wherein each of the motifs has 12 amino acid residues consisting of glycine (G), alanine (A), serine (S), threonine (T), glutamate (E) and proline (P), and wherein the content of any one amino acid type in the full-length XTEN does not exceed 30%.

In still other embodiments, XTENs comprise non-repetitive sequences of greater than about 100 to about 3000 amino acid residues, preferably greater than 400 to about 3000 amino acid residues wherein at least about 80%, or at least about 90%, or about 91%, or about 92%, or about 93%, or about 94%, or about 95%, or about 96%, or about 97%, or about 98%, or about 99% of the sequence consists of non-overlapping sequence motifs of 9 to 14 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in any one motif is not repeated more than twice in the sequence motif. In other embodiments, at least about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in any one sequence motif is not repeated more than twice in the sequence motif. In other embodiments, at least about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of glycine (G), alanine (A), serine (S), threonine (T), glutamate (E) and proline (P), and wherein the sequence of any two contiguous amino acid residues in any one sequence motif is not repeated more than twice in the sequence motif. In yet other embodiments, XTENs consist of 12 amino acid sequence motifs wherein the amino acids are selected from glycine (G), alanine (A), serine (S), threonine (T), glutamate (E) and proline (P), and wherein the sequence of any two contiguous amino acid residues in any one sequence motif is not repeated more than twice in the sequence motif, and wherein the content of any one amino acid type in the full-length XTEN does not exceed 30%. In the foregoing embodiments hereinabove described in this paragraph, the XTEN sequences would be substantially non-repetitive.

In some embodiments, the invention provides compositions comprising non-repetitive XTEN sequence(s) of greater than about 100 to about 3000 amino acid residues, of cumulatively greater than 400 to about 3000 residues, wherein at least about 80%, or at least about 90%, or about 91%, or about 92%, or about 93%, or about 94%, or about 95%, or about 96%, or about 97%, or about 98%, or about 99% to about 100% of the sequence consists of multiple units of two or more non-overlapping sequence motifs selected from the amino acid sequences of Table 2. In some embodiments, the XTEN comprises non-overlapping sequence motifs in which about 80%, or at least about 90%, or about 91%, or about 92%, or about 93%, or about 94%, or about 95%, or about 96%, or about 97%, or about 98%, or about 99% to about 100% of the sequence consists of two or more non-overlapping sequences selected from a single motif family of Table 2, resulting in a “family” sequence in which the overall sequence remains substantially non-repetitive. Accordingly, in these embodiments, an XTEN sequence comprises multiple units of non-overlapping sequence motifs of the AD motif family, or the AE motif family, or the AF motif family, or the AG motif family, or the AM motif family of sequences of Table 2. In other embodiments, the XTEN comprises motif sequences from two or more of the motif families of Table 2. In other embodiments, the XTEN comprises motif sequences from two or more of the motif families of Table 2.

TABLE 2
XTEN Sequence Motifs of 12 Amino Acids and
Motif Families

| Motif | SEQ ID | |
|---|---|---|
| Family* | NO: | MOTIF SEQUENCE |
| AD | 15 | GESPGGSSGSES |
| AD | 16 | GSEGSSGPGESS |
| AD | 17 | GSSESGSSEGGP |
| AD | 18 | GSGGEPSESGSS |
| AE, AM | 19 | GSPAGSPTSTEE |
| AE, AM, AQ | 20 | GSEPATSGSETP |
| AE, AM, AQ | 21 | GTSESATPESGP |
| AE, AM, AQ | 22 | GTSTEPSEGSAP |
| AF, AM | 23 | GSTSESPSGTAP |
| AF, AM | 24 | GTSTPESGSASP |
| AF, AM | 25 | GTSPSGESSTAP |
| AF, AM | 26 | GSTSSTAESPGP |
| AG, AM | 27 | GTPGSGTASSSP |
| AG, AM | 28 | GSSTPSGATGSP |
| AG, AM | 29 | GSSPSASTGTGP |
| AG, AM | 30 | GASPGTSSTGSP |

Denotes individual motif sequences that, when used together in various permutations, results in a “family sequence”

In other embodiments, the hGH-XTEN composition comprises a non-repetitive XTEN sequence of greater than about 100 to about 3000 amino acid residues, preferably greater than 400 to about 3000 residues, wherein at least about 80%, or at least about 90%, or about 91%, or about 92%, or about 93%, or about 94%, or about 95%, or about 96%, or about 97%, or about 98%, or about 99% to about 100% of the sequence consists of non-overlapping 36 amino acid sequence motifs selected from one or more of the polypeptide sequences of Tables 8-11 of Schellenberger et al. WO10/144502A2 (which is incorporated herein by reference in its entirety).

In those embodiments wherein the XTEN component of the hGH-XTEN fusion protein has less than 100% of its amino acids consisting of four to six amino acid selected from glycine (G), alanine (A), serine (S), threonine (T), glutamate (E) and proline (P), or less than 100% of the sequence consisting of the sequence motifs of Table 2, or less than 100% sequence identity with an XTEN from Table 3, the other amino acid residues are selected from any other of the 14 natural L-amino acids, but are preferentially selected from hydrophilic amino acids such that the XTEN sequence contains at least about 90%, or at least about 91%, or at least about 92%, or at least about 93%, or at least about 94%, or at least about 95%, or at least about 96%, or at least about 97%, or at least about 98%, or at least about 99% hydrophilic amino acids. The XTEN amino acids that are not glycine (G), alanine (A), serine (S), threonine (T), glutamate (E) and proline (P) are interspersed throughout the XTEN sequence, are located within or between the sequence motifs, or are concentrated in one or more short stretches of the XTEN sequence. In such cases where the XTEN component of the hGH-XTEN comprises amino acids other than glycine (G), alanine (A), serine (S), threonine (T), glutamate (E) and proline (P), it is preferred that the amino acids not be hydrophobic residues and should not substantially confer secondary structure of the XTEN component. Hydrophobic residues that are less favored in construction of XTEN include tryptophan, phenylalanine, tyrosine, leucine, isoleucine, valine, and methionine. Additionally, one can design the XTEN sequences to contain few (e.g. less than 5%) or none of the following amino acids: cysteine (to avoid disulfide formation and oxidation), methionine (to avoid oxidation), asparagine and glutamine (to avoid desamidation). Thus, in some embodiments, the XTEN component of the hGH-XTEN fusion protein comprising other amino acids in addition to glycine (G), alanine (A), serine (S), threonine (T), glutamate (E) and proline (P) would have a sequence with less than 5% of the residues contributing to alpha-helices and beta-sheets as measured by the Chou-Fasman algorithm and have at least 90%, or at least about 95% or more random coil formation as measured by the GOR algorithm.

3. Length of Sequence

In another aspect of the present invention, the invention encompasses hGH-XTEN compositions comprising carriers of XTEN polypeptides with extended length sequences. The present invention makes use of the discovery that increasing the length of non-repetitive, unstructured polypeptides enhances the unstructured nature of the XTENs and correspondingly enhances the biological and pharmacokinetic properties of fusion proteins comprising the XTEN carrier. As described more fully in the Examples, proportional increases in the length of the XTEN, even if created by a fixed repeat order of single family sequence motifs (e.g., the four AE motifs of Table 2), result in a sequence with a higher percentage of random coil formation, as determined by GOR algorithm, compared to shorter XTEN lengths. In general, increasing the length of the unstructured polypeptide fusion partner, as described in the Examples, results in a fusion protein with a disproportional increase in terminal half-life compared to fusion proteins with unstructured polypeptide partners with shorter sequence lengths.

Non-limiting examples of XTEN contemplated for inclusion in the hGH-XTEN of the invention are presented in Table 3. In one embodiment, the invention provides hGH-XTEN compositions wherein the XTEN sequence length of the fusion protein(s) is greater than about 100 to about 3000 amino acid residues, and in some cases is greater than 400 to about 3000 amino acid residues, wherein the XTEN confers enhanced pharmacokinetic properties on the hGH-XTEN in comparison to GH not linked to XTEN. In some embodiments, the XTEN sequences of the hGH-XTEN compositions of the present invention can be about 100, or about 144, or about 288, or about 401, or about 500, or about 600, or about 700, or about 800, or about 900, or about 1000, or about 1500, or about 2000, or about 2500 or up to about 3000 amino acid residues in length. In other cases, the XTEN sequences can be about 100 to 150, about 150 to 250, about 250 to 400, 401 to about 500, about 500 to 900, about 900 to 1500, about 1500 to 2000, or about 2000 to about 3000 amino acid residues in length. In one embodiment, the hGH-XTEN can comprise an XTEN sequence wherein the sequence exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to a XTEN selected from Table 3. In some embodiments, the XTEN sequence is designed for optimized expression as the N-terminal component of the hGH-XTEN by inclusion of encoding nucleotides for an optimized N-terminal leader sequence (NTS) in the XTEN portion of the gene encoding the fusion protein. In another embodiment, the N-terminal XTEN sequence of the expressed hGH-XTEN has at least 90% sequence identity to any sequence selected from Table 3. In one embodiment, the N-terminal XTEN sequence of the expressed hGH-XTEN has at least 90% sequence identity to the sequence of AE48 or AM48, AE624, AE911, AE912 or AM923.

In other embodiments, the hGH-XTEN fusion protein comprises a first and a second XTEN sequence, wherein the cumulative total of the residues in the XTEN sequences is greater than about 400 to about 3000 amino acid residues. In embodiments of the foregoing, the hGH-XTEN fusion protein comprises a first and a second XTEN sequence wherein the sequences each exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to at least a first or additionally a second XTEN selected from Table 3. Examples where more than one XTEN is used in a hGH-XTEN composition include, but are not limited to constructs with an XTEN linked to both the N- and C-termini of at least one GH.

As described more fully below, the invention provides methods in which the hGH-XTEN is designed by selecting the length of the XTEN to confer a target half-life on a fusion protein administered to a subject. In general, XTEN lengths longer that about cumulative 400 residues incorporated into the hGH-XTEN compositions result in longer half-life compared to shorter cumulative lengths; e.g., shorter than about 280 residues. However, in another embodiment, hGH-XTEN fusion proteins are designed to comprise XTEN with a longer sequence length that is selected to additionally confer slower rates of systemic absorption after subcutaneous or intramuscular administration to a subject. In such embodiments, the Cmaxis reduced in comparison to a comparable dose of a GH not linked to XTEN, thereby contributing to the ability to keep the hGH-XTEN within the therapeutic window for the composition. Thus, the XTEN confers the property of a depot to the administered hGH-XTEN, in addition to the other physical/chemical properties described herein.

TABLE 3
XTEN Polypeptides

| XTEN | SEQ | |
|---|---|---|
| Name | ID NO: | Amino Acid Sequence |
| AE48 | 31 | MAEPAGSPTSTEEGTPGSGTASSSPGSSTPSGATGSPGASPGTSSTGS |
| AM48 | 32 | MAEPAGSPTSTEEGASPGTSSTGSPGSSTPSGATGSPGSSTPSGATGS |
| AE144 | 33 | GSEPATSGSETPGTSESATPESGPGSEPATSGSETPGSPAGSPTSTEEGTSTEPSEGSA |
| | | PGSEPATSGSETPGSEPATSGSETPGSEPATSGSETPGTSTEPSEGSAPGTSESATPES |
| | | GPGSEPATSGSETPGTSTEPSEGSAP |
| AF144 | 34 | GTSTPESGSASPGTSPSGESSTAPGTSPSGESSTAPGSTSSTAESPGPGSTSESPSGTA |
| | | PGSTSSTAESPGPGTSPSGESSTAPGTSTPESGSASPGSTSSTAESPGPGTSPSGESST |
| | | APGTSPSGESSTAPGTSPSGESSTAP |
| AE288 | 35 | GTSESATPESGPGSEPATSGSETPGTSESATPESGPGSEPATSGSETPGTSESATPESG |
| | | PGTSTEPSEGSAPGSPAGSPTSTEEGTSESATPESGPGSEPATSGSETPGTSESATPES |
| | | GPGSPAGSPTSTEEGSPAGSPTSTEEGTSTEPSEGSAPGTSESATPESGPGTSESATPE |
| | | SGPGTSESATPESGPGSEPATSGSETPGSEPATSGSETPGSPAGSPTSTEEGTSTEPSE |
| | | GSAPGTSTEPSEGSAPGSEPATSGSETPGTSESATPESGPGTSTEPSEGSAP |
| AF504 | 36 | GASPGTSSTGSPGSSPSASTGTGPGSSPSASTGTGPGTPGSGTASSSPGSSTPSGATG |
| | | SPGSXPSASTGTGPGASPGTSSTGSPGTPGSGTASSSPGSSTPSGATGSPGTPGSGTA |
| | | SSSPGASPGTSSTGSPGASPGTSSTGSPGTPGSGTASSSPGSSTPSGATGSPGASPGTS |
| | | STGSPGTPGSGTASSSPGSSTPSGATGSPGSXPSASTGTGPGSSPSASTGTGPGSSTPS |
| | | GATGSPGSSTPSGATGSPGASPGTSSTGSPGASPGTSSTGSPGASPGTSSTGSPGTPG |
| | | SGTASSSPGASPGTSSTGSPGASPGTSSTGSPGASPGTSSTGSPGSSPSASTGTGPGTP |
| | | GSGTASSSPGASPGTSSTGSPGASPGTSSTGSPGASPGTSSTGSPGSSTPSGATGSPGS |
| | | STPSGATGSPGASPGTSSTGSPGTPGSGTASSSPGSSTPSGATGSPGSSTPSGATGSP |
| | | GSSTPSGATGSPGSSPSASTGTGPGASPGTSSTGSP |
| AF540 | 37 | GSTSSTAESPGPGSTSSTAESPGPGSTSESPSGTAPGSTSSTAESPGPGSTSSTAESPG |
| | | PGTSTPESGSASPGSTSESPSGTAPGTSPSGESSTAPGSTSESPSGTAPGSTSESPSGT |
| | | APGTSPSGESSTAPGSTSESPSGTAPGSTSESPSGTAPGTSPSGESSTAPGSTSESPSG |
| | | TAPGSTSESPSGTAPGSTSESPSGTAPGTSTPESGSASPGSTSESPSGTAPGTSTPESG |
| | | SASPGSTSSTAESPGPGSTSSTAESPGPGTSTPESGSASPGTSTPESGSASPGSTSESPS |
| | | GTAPGTSTPESGSASPGTSTPESGSASPGSTSESPSGTAPGSTSESPSGTAPGSTSESP |
| | | SGTAPGSTSSTAESPGPGTSTPESGSASPGTSTPESGSASPGSTSESPSGTAPGSTSES |
| | | PSGTAPGTSTPESGSASPGSTSESPSGTAPGSTSESPSGTAPGTSTPESGSASPGTSPS |
| | | GESSTAPGSTSSTAESPGPGTSPSGESSTAPGSTSSTAESPGPGTSTPESGSASPGSTS |
| | | ESPSGTAP |
| AD576 | 38 | GSSESGSSEGGPGSGGEPSESGSSGSSESGSSEGGPGSSESGSSEGGPGSSESGSSEG |
| | | GPGSSESGSSEGGPGSSESGSSEGGPGESPGGSSGSESGSEGSSGPGESSGSSESGSSE |
| | | GGPGSSESGSSEGGPGSSESGSSEGGPGSGGEPSESGSSGESPGGSSGSESGESPGGS |
| | | SGSESGSGGEPSESGSSGSSESGSSEGGPGSGGEPSESGSSGSGGEPSESGSSGSEGSS |
| | | GPGESSGESPGGSSGSESGSGGEPSESGSSGSGGEPSESGSSGSGGEPSESGSSGSSES |
| | | GSSEGGPGESPGGSSGSESGESPGGSSGSESGESPGGSSGSESGESPGGSSGSESGES |
| | | PGGSSGSESGSSESGSSEGGPGSGGEPSESGSSGSEGSSGPGESSGSSESGSSEGGPGS |
| | | GGEPSESGSSGSSESGSSEGGPGSGGEPSESGSSGESPGGSSGSESGESPGGSSGSES |
| | | GSSESGSSEGGPGSGGEPSESGSSGSSESGSSEGGPGSGGEPSESGSSGSGGEPSESG |
| | | SSGESPGGSSGSESGSEGSSGPGESSGSSESGSSEGGPGSEGSSGPGESS |
| AE576 | 39 | GSPAGSPTSTEEGTSESATPESGPGTSTEPSEGSAPGSPAGSPTSTEEGTSTEPSEGSA |
| | | PGTSTEPSEGSAPGTSESATPESGPGSEPATSGSETPGSEPATSGSETPGSPAGSPTST |
| | | EEGTSESATPESGPGTSTEPSEGSAPGTSTEPSEGSAPGSPAGSPTSTEEGTSTEPSEG |
| | | SAPGTSTEPSEGSAPGTSESATPESGPGTSTEPSEGSAPGTSESATPESGPGSEPATSG |
| | | SETPGTSTEPSEGSAPGTSTEPSEGSAPGTSESATPESGPGTSESATPESGPGSPAGSP |
| | | TSTEEGTSESATPESGPGSEPATSGSETPGTSESATPESGPGTSTEPSEGSAPGTSTEP |
| | | SEGSAPGTSTEPSEGSAPGTSTEPSEGSAPGTSTEPSEGSAPGTSTEPSEGSAPGSPAG |
| | | SPTSTEEGTSTEPSEGSAPGTSESATPESGPGSEPATSGSETPGTSESATPESGPGSEP |
| | | ATSGSETPGTSESATPESGPGTSTEPSEGSAPGTSESATPESGPGSPAGSPTSTEEGSP |
| | | AGSPTSTEEGSPAGSPTSTEEGTSESATPESGPGTSTEPSEGSAP |
| AF576 | 40 | GSTSSTAESPGPGSTSSTAESPGPGSTSESPSGTAPGSTSSTAESPGPGSTSSTAESPG |
| | | PGTSTPESGSASPGSTSESPSGTAPGTSPSGESSTAPGSTSESPSGTAPGSTSESPSGT |
| | | APGTSPSGESSTAPGSTSESPSGTAPGSTSESPSGTAPGTSPSGESSTAPGSTSESPSG |
| | | TAPGSTSESPSGTAPGSTSESPSGTAPGTSTPESGSASPGSTSESPSGTAPGTSTPESG |
| | | SASPGSTSSTAESPGPGSTSSTAESPGPGTSTPESGSASPGTSTPESGSASPGSTSESPS |
| | | GTAPGTSTPESGSASPGTSTPESGSASPGSTSESPSGTAPGSTSESPSGTAPGSTSESP |
| | | SGTAPGSTSSTAESPGPGTSTPESGSASPGTSTPESGSASPGSTSESPSGTAPGSTSES |
| | | PSGTAPGTSTPESGSASPGSTSESPSGTAPGSTSESPSGTAPGTSTPESGSASPGTSPS |
| | | GESSTAPGSTSSTAESPGPGTSPSGESSTAPGSTSSTAESPGPGTSTPESGSASPGSTS |
| | | ESPSGTAPGSTSSTAESPGPGTSTPESGSASPGTSTPESGSASP |
| AE624 | 41 | MAEPAGSPTSTEEGTPGSGTASSSPGSSTPSGATGSPGASPGTSSTGSPGSPAGSPTS |
| | | TEEGTSESATPESGPGTSTEPSEGSAPGSPAGSPTSTEEGTSTEPSEGSAPGTSTEPSE |
| | | GSAPGTSESATPESGPGSEPATSGSETPGSEPATSGSETPGSPAGSPTSTEEGTSESAT |
| | | PESGPGTSTEPSEGSAPGTSTEPSEGSAPGSPAGSPTSTEEGTSTEPSEGSAPGTSTEP |
| | | SEGSAPGTSESATPESGPGTSTEPSEGSAPGTSESATPESGPGSEPATSGSETPGTSTE |
| | | PSEGSAPGTSTEPSEGSAPGTSESATPESGPGTSESATPESGPGSPAGSPTSTEEGTSE |
| | | SATPESGPGSEPATSGSETPGTSESATPESGPGTSTEPSEGSAPGTSTEPSEGSAPGTS |
| | | TEPSEGSAPGTSTEPSEGSAPGTSTEPSEGSAPGTSTEPSEGSAPGSPAGSPTSTEEGT |
| | | STEPSEGSAPGTSESATPESGPGSEPATSGSETPGTSESATPESGPGSEPATSGSETPG |
| | | TSESATPESGPGTSTEPSEGSAPGTSESATPESGPGSPAGSPTSTEEGSPAGSPTSTEE |
| | | GSPAGSPTSTEEGTSESATPESGPGTSTEPSEGSAP |
| AD836 | 42 | GSSESGSSEGGPGSSESGSSEGGPGESPGGSSGSESGSGGEPSESGSSGESPGGSSGS |
| | | ESGESPGGSSGSESGSSESGSSEGGPGSSESGSSEGGPGSSESGSSEGGPGESPGGSS |
| | | GSESGESPGGSSGSESGESPGGSSGSESGSSESGSSEGGPGSSESGSSEGGPGSSESGS |
| | | SEGGPGSSESGSSEGGPGSSESGSSEGGPGSSESGSSEGGPGSGGEPSESGSSGESPG |
| | | GSSGSESGESPGGSSGSESGSGGEPSESGSSGSEGSSGPGESSGSSESGSSEGGPGSG |
| | | GEPSESGSSGSEGSSGPGESSGSSESGSSEGGPGSGGEPSESGSSGESPGGSSGSESGS |
| | | GGEPSESGSSGSGGEPSESGSSGSSESGSSEGGPGSGGEPSESGSSGSGGEPSESGSS |
| | | GSEGSSGPGESSGESPGGSSGSESGSEGSSGPGESSGSEGSSGPGESSGSGGEPSESG |
| | | SSGSSESGSSEGGPGSSESGSSEGGPGESPGGSSGSESGSGGEPSESGSSGSEGSSGPG |
| | | ESSGESPGGSSGSESGSEGSSGPGSSESGSSEGGPGSGGEPSESGSSGSEGSSGPGESS |
| | | GSEGSSGPGESSGSEGSSGPGESSGSGGEPSESGSSGSGGEPSESGSSGESPGGSSGS |
| | | ESGESPGGSSGSESGSGGEPSESGSSGSEGSSGPGESSGESPGGSSGSESGSSESGSSE |
| | | GGPGSSESGSSEGGPGSSESGSSEGGPGSGGEPSESGSSGSSESGSSEGGPGESPGGS |
| | | SGSESGSGGEPSESGSSGSSESGSSEGGPGESPGGSSGSESGSGGEPSESGSSGESPG |
| | | GSSGSESGSGGEPSESGSS |
| AE864 | 43 | GSPAGSPTSTEEGTSESATPESGPGTSTEPSEGSAPGSPAGSPTSTEEGTSTEPSEGSA |
| | | PGTSTEPSEGSAPGTSESATPESGPGSEPATSGSETPGSEPATSGSETPGSPAGSPTST |
| | | EEGTSESATPESGPGTSTEPSEGSAPGTSTEPSEGSAPGSPAGSPTSTEEGTSTEPSEG |
| | | SAPGTSTEPSEGSAPGTSESATPESGPGTSTEPSEGSAPGTSESATPESGPGSEPATSG |
| | | SETPGTSTEPSEGSAPGTSTEPSEGSAPGTSESATPESGPGTSESATPESGPGSPAGSP |
| | | TSTEEGTSESATPESGPGSEPATSGSETPGTSESATPESGPGTSTEPSEGSAPGTSTEP |
| | | SEGSAPGTSTEPSEGSAPGTSTEPSEGSAPGTSTEPSEGSAPGTSTEPSEGSAPGSPAG |
| | | SPTSTEEGTSTEPSEGSAPGTSESATPESGPGSEPATSGSETPGTSESATPESGPGSEP |
| | | ATSGSETPGTSESATPESGPGTSTEPSEGSAPGTSESATPESGPGSPAGSPTSTEEGSP |
| | | AGSPTSTEEGSPAGSPTSTEEGTSESATPESGPGTSTEPSEGSAPGTSESATPESGPGS |
| | | EPATSGSETPGTSESATPESGPGSEPATSGSETPGTSESATPESGPGTSTEPSEGSAPG |
| | | SPAGSPTSTEEGTSESATPESGPGSEPATSGSETPGTSESATPESGPGSPAGSPTSTEE |
| | | GSPAGSPTSTEEGTSTEPSEGSAPGTSESATPESGPGTSESATPESGPGTSESATPESG |
| | | PGSEPATSGSETPGSEPATSGSETPGSPAGSPTSTEEGTSTEPSEGSAPGTSTEPSEGS |
| | | APGSEPATSGSETPGTSESATPESGPGTSTEPSEGSAP |
| AF864 | 44 | GSTSESPSGTAPGTSPSGESSTAPGSTSESPSGTAPGSTSESPSGTAPGTSTPESGSAS |
| | | PGTSTPESGSASPGSTSESPSGTAPGSTSESPSGTAPGTSPSGESSTAPGSTSESPSGT |
| | | APGTSPSGESSTAPGTSPSGESSTAPGSTSSTAESPGPGTSPSGESSTAPGTSPSGESS |
| | | TAPGSTSSTAESPGPGTSTPESGSASPGTSTPESGSASPGSTSESPSGTAPGSTSESPS |
| | | GTAPGTSTPESGSASPGSTSSTAESPGPGTSTPESGSASPGSTSESPSGTAPGTSPSGE |
| | | SSTAPGSTSSTAESPGPGTSPSGESSTAPGTSTPESGSASPGSTSSTAESPGPGSTSST |
| | | AESPGPGSTSSTAESPGPGSTSSTAESPGPGTSPSGESSTAPGSTSESPSGTAPGSTSE |
| | | SPSGTAPGTSTPESGPXXXGASASGAPSTXXXXSESPSGTAPGSTSESPSGTAPGSTS |
| | | ESPSGTAPGSTSESPSGTAPGSTSESPSGTAPGSTSESPSGTAPGTSTPESGSASPGTS |
| | | PSGESSTAPGTSPSGESSTAPGSTSSTAESPGPGTSPSGESSTAPGTSTPESGSASPGS |
| | | TSESPSGTAPGSTSESPSGTAPGTSPSGESSTAPGSTSESPSGTAPGTSTPESGSASPG |
| | | TSTPESGSASPGSTSESPSGTAPGTSTPESGSASPGSTSSTAESPGPGSTSESPSGTAP |
| | | GSTSESPSGTAPGTSPSGESSTAPGSTSSTAESPGPGTSPSGESSTAPGTSTPESGSAS |
| | | PGTSPSGESSTAPGTSPSGESSTAPGTSPSGESSTAPGSTSSTAESPGPGSTSSTAESP |
| | | GPGTSPSGESSTAPGSSPSASTGTGPGSSTPSGATGSPGSSTPSGATGSP |
| AG864 | 45 | GASPGTSSTGSPGSSPSASTGTGPGSSPSASTGTGPGTPGSGTASSSPGSSTPSGATG |
| | | SPGSSPSASTGTGPGASPGTSSTGSPGTPGSGTASSSPGSSTPSGATGSPGTPGSGTA |
| | | SSSPGASPGTSSTGSPGASPGTSSTGSPGTPGSGTASSSPGSSTPSGATGSPGASPGTS |
| | | STGSPGTPGSGTASSSPGSSTPSGATGSPGSSPSASTGTGPGSSPSASTGTGPGSSTPS |
| | | GATGSPGSSTPSGATGSPGASPGTSSTGSPGASPGTSSTGSPGASPGTSSTGSPGTPG |
| | | SGTASSSPGASPGTSSTGSPGASPGTSSTGSPGASPGTSSTGSPGSSPSASTGTGPGTP |
| | | GSGTASSSPGASPGTSSTGSPGASPGTSSTGSPGASPGTSSTGSPGSSTPSGATGSPGS |
| | | STPSGATGSPGASPGTSSTGSPGTPGSGTASSSPGSSTPSGATGSPGSSTPSGATGSP |
| | | GSSTPSGATGSPGSSPSASTGTGPGASPGTSSTGSPGASPGTSSTGSPGTPGSGTASS |
| | | SPGASPGTSSTGSPGASPGTSSTGSPGASPGTSSTGSPGASPGTSSTGSPGTPGSGTA |
| | | SSSPGSSTPSGATGSPGTPGSGTASSSPGSSTPSGATGSPGTPGSGTASSSPGSSTPSG |
| | | ATGSPGSSTPSGATGSPGSSPSASTGTGPGSSPSASTGTGPGASPGTSSTGSPGTPGS |
| | | GTASSSPGSSTPSGATGSPGSSPSASTGTGPGSSPSASTGTGPGASPGTSSTGSPGASP |
| | | GTSSTGSPGSSTPSGATGSPGSSPSASTGTGPGASPGTSSTGSPGSSPSASTGTGPGTP |
| | | GSGTASSSPGSSTPSGATGSPGSSTPSGATGSPGASPGTSSTGSP |
| AM875 | 46 | GTSTEPSEGSAPGSEPATSGSETPGSPAGSPTSTEEGSTSSTAESPGPGTSTPESGSAS |
| | | PGSTSESPSGTAPGSTSESPSGTAPGTSTPESGSASPGTSTPESGSASPGSEPATSGSE |
| | | TPGTSESATPESGPGSPAGSPTSTEEGTSTEPSEGSAPGTSESATPESGPGTSTEPSEG |
| | | SAPGTSTEPSEGSAPGSPAGSPTSTEEGTSTEPSEGSAPGTSTEPSEGSAPGTSESATP |
| | | ESGPGTSESATPESGPGTSTEPSEGSAPGTSTEPSEGSAPGTSESATPESGPGTSTEPS |
| | | EGSAPGSEPATSGSETPGSPAGSPTSTEEGSSTPSGATGSPGTPGSGTASSSPGSSTPS |
| | | GATGSPGTSTEPSEGSAPGTSTEPSEGSAPGSEPATSGSETPGSPAGSPTSTEEGSPA |
| | | GSPTSTEEGTSTEPSEGSAPGASASGAPSTGGTSESATPESGPGSPAGSPTSTEEGSP |
| | | AGSPTSTEEGSTSSTAESPGPGSTSESPSGTAPGTSPSGESSTAPGTPGSGTASSSPGS |
| | | STPSGATGSPGSSPSASTGTGPGSEPATSGSETPGTSESATPESGPGSEPATSGSETPG |
| | | STSSTAESPGPGSTSSTAESPGPGTSPSGESSTAPGSEPATSGSETPGSEPATSGSETP |
| | | GTSTEPSEGSAPGSTSSTAESPGPGTSTPESGSASPGSTSESPSGTAPGTSTEPSEGSA |
| | | PGTSTEPSEGSAPGTSTEPSEGSAPGSSTPSGATGSPGSSPSASTGTGPGASPGTSSTG |
| | | SPGSEPATSGSETPGTSESATPESGPGSPAGSPTSTEEGSSTPSGATGSPGSSPSASTG |
| | | TGPGASPGTSSTGSPGTSESATPESGPGTSTEPSEGSAPGTSTEPSEGSAP |
| AE912 | 47 | MAEPAGSPTSTEEGTPGSGTASSSPGSSTPSGATGSPGASPGTSSTGSPGSPAGSPTS |
| | | TEEGTSESATPESGPGTSTEPSEGSAPGSPAGSPTSTEEGTSTEPSEGSAPGTSTEPSE |
| | | GSAPGTSESATPESGPGSEPATSGSETPGSEPATSGSETPGSPAGSPTSTEEGTSESAT |
| | | PESGPGTSTEPSEGSAPGTSTEPSEGSAPGSPAGSPTSTEEGTSTEPSEGSAPGTSTEP |
| | | SEGSAPGTSESATPESGPGTSTEPSEGSAPGTSESATPESGPGSEPATSGSETPGTSTE |
| | | PSEGSAPGTSTEPSEGSAPGTSESATPESGPGTSESATPESGPGSPAGSPTSTEEGTSE |
| | | SATPESGPGSEPATSGSETPGTSESATPESGPGTSTEPSEGSAPGTSTEPSEGSAPGTS |
| | | TEPSEGSAPGTSTEPSEGSAPGTSTEPSEGSAPGTSTEPSEGSAPGSPAGSPTSTEEGT |
| | | STEPSEGSAPGTSESATPESGPGSEPATSGSETPGTSESATPESGPGSEPATSGSETPG |
| | | TSESATPESGPGTSTEPSEGSAPGTSESATPESGPGSPAGSPTSTEEGSPAGSPTSTEE |
| | | GSPAGSPTSTEEGTSESATPESGPGTSTEPSEGSAPGTSESATPESGPGSEPATSGSET |
| | | PGTSESATPESGPGSEPATSGSETPGTSESATPESGPGTSTEPSEGSAPGSPAGSPTST |
| | | EEGTSESATPESGPGSEPATSGSETPGTSESATPESGPGSPAGSPTSTEEGSPAGSPTS |
| | | TEEGTSTEPSEGSAPGTSESATPESGPGTSESATPESGPGTSESATPESGPGSEPATSG |
| | | SETPGSEPATSGSETPGSPAGSPTSTEEGTSTEPSEGSAPGTSTEPSEGSAPGSEPATS |
| | | GSETPGTSESATPESGPGTSTEPSEGSAP |
| AM923 | 48 | MAEPAGSPTSTEEGASPGTSSTGSPGSSTPSGATGSPGSSTPSGATGSPGTSTEPSEG |
| | | SAPGSEPATSGSETPGSPAGSPTSTEEGSTSSTAESPGPGTSTPESGSASPGSTSESPS |
| | | GTAPGSTSESPSGTAPGTSTPESGSASPGTSTPESGSASPGSEPATSGSETPGTSESAT |
| | | PESGPGSPAGSPTSTEEGTSTEPSEGSAPGTSESATPESGPGTSTEPSEGSAPGTSTEP |
| | | SEGSAPGSPAGSPTSTEEGTSTEPSEGSAPGTSTEPSEGSAPGTSESATPESGPGTSES |
| | | ATPESGPGTSTEPSEGSAPGTSTEPSEGSAPGTSESATPESGPGTSTEPSEGSAPGSEP |
| | | ATSGSETPGSPAGSPTSTEEGSSTPSGATGSPGTPGSGTASSSPGSSTPSGATGSPGTS |
| | | TEPSEGSAPGTSTEPSEGSAPGSEPATSGSETPGSPAGSPTSTEEGSPAGSPTSTEEGT |
| | | STEPSEGSAPGASASGAPSTGGTSESATPESGPGSPAGSPTSTEEGSPAGSPTSTEEG |
| | | STSSTAESPGPGSTSESPSGTAPGTSPSGESSTAPGTPGSGTASSSPGSSTPSGATGSP |
| | | GSSPSASTGTGPGSEPATSGSETPGTSESATPESGPGSEPATSGSETPGSTSSTAESPG |
| | | PGSTSSTAESPGPGTSPSGESSTAPGSEPATSGSETPGSEPATSGSETPGTSTEPSEGS |
| | | APGSTSSTAESPGPGTSTPESGSASPGSTSESPSGTAPGTSTEPSEGSAPGTSTEPSEG |
| | | SAPGTSTEPSEGSAPGSSTPSGATGSPGSSPSASTGTGPGASPGTSSTGSPGSEPATSG |
| | | SETPGTSESATPESGPGSPAGSPTSTEEGSSTPSGATGSPGSSPSASTGTGPGASPGTS |
| | | STGSPGTSESATPESGPGTSTEPSEGSAPGTSTEPSEGSAP |
| AM1318 | 49 | GTSTEPSEGSAPGSEPATSGSETPGSPAGSPTSTEEGSTSSTAESPGPGTSTPESGSAS |
| | | PGSTSESPSGTAPGSTSESPSGTAPGTSTPESGSASPGTSTPESGSASPGSEPATSGSE |
| | | TPGTSESATPESGPGSPAGSPTSTEEGTSTEPSEGSAPGTSESATPESGPGTSTEPSEG |
| | | SAPGTSTEPSEGSAPGSPAGSPTSTEEGTSTEPSEGSAPGTSTEPSEGSAPGTSESATP |
| | | ESGPGTSESATPESGPGTSTEPSEGSAPGTSTEPSEGSAPGTSESATPESGPGTSTEPS |
| | | EGSAPGSEPATSGSETPGSPAGSPTSTEEGSSTPSGATGSPGTPGSGTASSSPGSSTPS |
| | | GATGSPGTSTEPSEGSAPGTSTEPSEGSAPGSEPATSGSETPGSPAGSPTSTEEGSPA |
| | | GSPTSTEEGTSTEPSEGSAPGPEPTGPAPSGGSEPATSGSETPGTSESATPESGPGSPA |
| | | GSPTSTEEGTSESATPESGPGSPAGSPTSTEEGSPAGSPTSTEEGTSESATPESGPGSP |
| | | AGSPTSTEEGSPAGSPTSTEEGSTSSTAESPGPGSTSESPSGTAPGTSPSGESSTAPGS |
| | | TSESPSGTAPGSTSESPSGTAPGTSPSGESSTAPGTSTEPSEGSAPGTSESATPESGPG |
| | | TSESATPESGPGSEPATSGSETPGTSESATPESGPGTSESATPESGPGTSTEPSEGSAP |
| | | GTSESATPESGPGTSTEPSEGSAPGTSPSGESSTAPGTSPSGESSTAPGTSPSGESSTA |
| | | PGTSTEPSEGSAPGSPAGSPTSTEEGTSTEPSEGSAPGSSPSASTGTGPGSSTPSGATG |
| | | SPGSSTPSGATGSPGSSTPSGATGSPGSSTPSGATGSPGASPGTSSTGSPGASASGAP |
| | | STGGTSPSGESSTAPGSTSSTAESPGPGTSPSGESSTAPGTSESATPESGPGTSTEPSE |
| | | GSAPGTSTEPSEGSAPGSSPSASTGTGPGSSTPSGATGSPGASPGTSSTGSPGTSTPES |
| | | GSASPGTSPSGESSTAPGTSPSGESSTAPGTSESATPESGPGSEPATSGSETPGTSTEP |
| | | SEGSAPGSTSESPSGTAPGSTSESPSGTAPGTSTPESGSASPGSPAGSPTSTEEGTSES |
| | | ATPESGPGTSTEPSEGSAPGSPAGSPTSTEEGTSESATPESGPGSEPATSGSETPGSST |
| | | PSGATGSPGASPGTSSTGSPGSSTPSGATGSPGSTSESPSGTAPGTSPSGESSTAPGST |
| | | SSTAESPGPGSSTPSGATGSPGASPGTSSTGSPGTPGSGTASSSPGSPAGSPTSTEEGS |
| | | PAGSPTSTEEGTSTEPSEGSAP |
| BC 864 | 50 | GTSTEPSEPGSAGTSTEPSEPGSAGSEPATSGTEPSGSGASEPTSTEPGSEPATSGTEP |
| | | SGSEPATSGTEPSGSEPATSGTEPSGSGASEPTSTEPGTSTEPSEPGSAGSEPATSGTE |
| | | PSGTSTEPSEPGSAGSEPATSGTEPSGSEPATSGTEPSGTSTEPSEPGSAGTSTEPSEP |
| | | GSAGSEPATSGTEPSGSEPATSGTEPSGTSEPSTSEPGAGSGASEPTSTEPGTSEPSTS |
| | | EPGAGSEPATSGTEPSGSEPATSGTEPSGTSTEPSEPGSAGTSTEPSEPGSAGSGASEP |
| | | TSTEPGSEPATSGTEPSGSEPATSGTEPSGSEPATSGTEPSGSEPATSGTEPSGTSTEP |
| | | SEPGSAGSEPATSGTEPSGSGASEPTSTEPGTSTEPSEPGSAGSEPATSGTEPSGSGAS |
| | | EPTSTEPGTSTEPSEPGSAGSGASEPTSTEPGSEPATSGTEPSGSGASEPTSTEPGSEP |
| | | ATSGTEPSGSGASEPTSTEPGTSTEPSEPGSAGSEPATSGTEPSGSGASEPTSTEPGTS |
| | | TEPSEPGSAGSEPATSGTEPSGTSTEPSEPGSAGSEPATSGTEPSGTSTEPSEPGSAGT |
| | | STEPSEPGSAGTSTEPSEPGSAGTSTEPSEPGSAGTSTEPSEPGSAGTSTEPSEPGSAG |
| | | TSEPSTSEPGAGSGASEPTSTEPGTSTEPSEPGSAGTSTEPSEPGSAGTSTEPSEPGSA |
| | | GSEPATSGTEPSGSGASEPTSTEPGSEPATSGTEPSGSEPATSGTEPSGSEPATSGTEP |
| | | SGSEPATSGTEPSGTSEPSTSEPGAGSEPATSGTEPSGSGASEPTSTEPGTSTEPSEPG |
| | | SAGSEPATSGTEPSGSGASEPTSTEPGTSTEPSEPGSA |
| BD864 | 51 | GSETATSGSETAGTSESATSESGAGSTAGSETSTEAGTSESATSESGAGSETATSGSE |
| | | TAGSETATSGSETAGTSTEASEGSASGTSTEASEGSASGTSESATSESGAGSETATS |
| | | GSETAGTSTEASEGSASGSTAGSETSTEAGTSESATSESGAGTSESATSESGAGSET |
| | | ATSGSETAGTSESATSESGAGTSTEASEGSASGSETATSGSETAGSETATSGSETAG |
| | | TSTEASEGSASGSTAGSETSTEAGTSESATSESGAGTSTEASEGSASGSETATSGSET |
| | | AGSTAGSETSTEAGSTAGSETSTEAGSETATSGSETAGTSESATSESGAGTSESATS |
| | | ESGAGSETATSGSETAGTSESATSESGAGTSESATSESGAGSETATSGSETAGSETA |
| | | TSGSETAGTSTEASEGSASGSTAGSETSTEAGSETATSGSETAGTSESATSESGAGST |
| | | AGSETSTEAGSTAGSETSTEAGSTAGSETSTEAGTSTEASEGSASGSTAGSETSTEA |
| | | GSTAGSETSTEAGTSTEASEGSASGSTAGSETSTEAGSETATSGSETAGTSTEASEG |
| | | SASGTSESATSESGAGSETATSGSETAGTSESATSESGAGTSESATSESGAGSETATS |
| | | GSETAGTSESATSESGAGSETATSGSETAGTSTEASEGSASGTSTEASEGSASGSTA |
| | | GSETSTEAGSTAGSETSTEAGSETATSGSETAGTSESATSESGAGTSESATSESGAGS |
| | | ETATSGSETAGSETATSGSETAGSETATSGSETAGTSTEASEGSASGTSESATSESG |
| | | AGSETATSGSETAGSETATSGSETAGTSESATSESGAGTSESATSESGAGSETATSG |
| | | SETA |
| AE911 | 52 | AEPAGSPTSTEEGTPGSGTASSSPGSSTPSGATGSPGASPGTSSTGSPGSPAGSPTSTE |
| | | EGTSESATPESGPGTSTEPSEGSAPGSPAGSPTSTEEGTSTEPSEGSAPGTSTEPSEGS |
| | | APGTSESATPESGPGSEPATSGSETPGSEPATSGSETPGSPAGSPTSTEEGTSESATPE |
| | | SGPGTSTEPSEGSAPGTSTEPSEGSAPGSPAGSPTSTEEGTSTEPSEGSAPGTSTEPSE |
| | | GSAPGTSESATPESGPGTSTEPSEGSAPGTSESATPESGPGSEPATSGSETPGTSTEPS |
| | | EGSAPGTSTEPSEGSAPGTSESATPESGPGTSESATPESGPGSPAGSPTSTEEGTSESA |
| | | TPESGPGSEPATSGSETPGTSESATPESGPGTSTEPSEGSAPGTSTEPSEGSAPGTSTE |
| | | PSEGSAPGTSTEPSEGSAPGTSTEPSEGSAPGTSTEPSEGSAPGSPAGSPTSTEEGTST |
| | | EPSEGSAPGTSESATPESGPGSEPATSGSETPGTSESATPESGPGSEPATSGSETPGTS |
| | | ESATPESGPGTSTEPSEGSAPGTSESATPESGPGSPAGSPTSTEEGSPAGSPTSTEEGS |
| | | PAGSPTSTEEGTSESATPESGPGTSTEPSEGSAPGTSESATPESGPGSEPATSGSETPG |
| | | TSESATPESGPGSEPATSGSETPGTSESATPESGPGTSTEPSEGSAPGSPAGSPTSTEE |
| | | GTSESATPESGPGSEPATSGSETPGTSESATPESGPGSPAGSPTSTEEGSPAGSPTSTE |
| | | EGTSTEPSEGSAPGTSESATPESGPGTSESATPESGPGTSESATPESGPGSEPATSGSE |
| | | TPGSEPATSGSETPGSPAGSPTSTEEGTSTEPSEGSAPGTSTEPSEGSAPGSEPATSGS |
| | | ETPGTSESATPESGPGTSTEPSEGSAP |
| AE146 | 53 | GGTSESATPESGPGTSTEPSEGSAPGTSTEPSEGSAPGTSESATPESGPGTSTEPSEGS |
| | | APGTSTEPSEGSAPGTSESATPESGPGTSTEPSEGSAPGTSTEPSEGSAPGTSTEPSEG |
| | | SAPGSPAGSPTSTEEGTSTEPSEGSAPG |
| AE48.1 | 81 | AEPAGSPTSTEEGTPGSGTASSSPGSSTPSGATGSPGASPGTSSTGS |
| AM48.1 | 82 | AEPAGSPTSTEEGASPGTSSTGSPGSSTPSGATGSPGSSTPSGATGS |
| AE912.1 | 83 | AEPAGSPTSTEEGTPGSGTASSSPGSSTPSGATGSPGASPGTSSTGSPGSPAGSPTSTE |
| | | EGTSESATPESGPGTSTEPSEGSAPGSPAGSPTSTEEGTSTEPSEGSAPGTSTEPSEGS |
| | | APGTSESATPESGPGSEPATSGSETPGSEPATSGSETPGSPAGSPTSTEEGTSESATPE |
| | | SGPGTSTEPSEGSAPGTSTEPSEGSAPGSPAGSPTSTEEGTSTEPSEGSAPGTSTEPSE |
| | | GSAPGTSESATPESGPGTSTEPSEGSAPGTSESATPESGPGSEPATSGSETPGTSTEPS |
| | | EGSAPGTSTEPSEGSAPGTSESATPESGPGTSESATPESGPGSPAGSPTSTEEGTSESA |
| | | TPESGPGSEPATSGSETPGTSESATPESGPGTSTEPSEGSAPGTSTEPSEGSAPGTSTE |
| | | PSEGSAPGTSTEPSEGSAPGTSTEPSEGSAPGTSTEPSEGSAPGSPAGSPTSTEEGTST |
| | | EPSEGSAPGTSESATPESGPGSEPATSGSETPGTSESATPESGPGSEPATSGSETPGTS |
| | | ESATPESGPGTSTEPSEGSAPGTSESATPESGPGSPAGSPTSTEEGSPAGSPTSTEEGS |
| | | PAGSPTSTEEGTSESATPESGPGTSTEPSEGSAPGTSESATPESGPGSEPATSGSETPG |
| | | TSESATPESGPGSEPATSGSETPGTSESATPESGPGTSTEPSEGSAPGSPAGSPTSTEE |
| | | GTSESATPESGPGSEPATSGSETPGTSESATPESGPGSPAGSPTSTEEGSPAGSPTSTE |
| | | EGTSTEPSEGSAPGTSESATPESGPGTSESATPESGPGTSESATPESGPGSEPATSGSE |
| | | TPGSEPATSGSETPGSPAGSPTSTEEGTSTEPSEGSAPGTSTEPSEGSAPGSEPATSGS |
| | | ETPGTSESATPESGPGTSTEPSEGSAP |
| AE912.2 | 84 | AEPAGSPTSTEEGTPGSGTASSSPGSSTPSGATGSPGASPGTSSTGSPGSPAGSPTSTE |
| | | EGTSESATPESGPGTSTEPSEGSAPGSPAGSPTSTEEGTSTEPSEGSAPGTSTEPSEGS |
| | | APGTSESATPESGPGSEPATSGSETPGSEPATSGSETPGSPAGSPTSTEEGTSESATPE |
| | | SGPGTSTEPSEGSAPGTSTEPSEGSAPGSPAGSPTSTEEGTSTEPSEGSAPGTSTEPSE |
| | | GSAPGTSESATPESGPGTSTEPSEGSAPGTSESATPESGPGSEPATSGSETPGTSTEPS |
| | | EGSAPGTSTEPSEGSAPGTSESATPESGPGTSESATPESGPGSPAGSPTSTEEGTSESA |
| | | TPESGPGSEPATSGSETPGTSESATPESGPGTSTEPSEGSAPGTSTEPSEGSAPGTSTE |
| | | PSEGSAPGTSTEPSEGSAPGTSTEPSEGSAPGTSTEPSEGSAPGSPAGSPTSTEEGTST |
| | | EPSEGSAPGTSESATPESGPGSEPATSGSETPGTSESATPESGPGSEPATSGSETPGTS |
| | | ESATPESGPGTSTEPSEGSAPGTSESATPESGPGSPAGSPTSTEEGSPAGSPTSTEEGS |
| | | PAGSPTSTEEGTSESATPESGPGTSTEPSEGSAPGTSESATPESGPGSEPATSGSETPG |
| | | TSESATPESGPGSEPATSGSETPGTSESATPESGPGTSTEPSEGSAPGSPAGSPTSTEE |
| | | GTSESATPESGPGSEPATSGSETPGTSESATPESGPGSPAGSPTSTEEGSPAGSPTSTE |
| | | EGTSTEPSEGSAPGTSESATPESGPGTSESATPESGPGTSESATPESGPGSEPATSGSE |
| | | TPGSEPATSGSETPGSPAGSPTSTEEGTSTEPSEGSAPGTSTEPSEGSAPGSEPATSGS |
| | | ETPGTSESATPESGPGTSTEPSEGSAPG |
| AE146.1 | 85 | TSESATPESGPGTSTEPSEGSAPGTSTEPSEGSAPGTSESATPESGPGTSTEPSEGSAP |
| | | GTSTEPSEGSAPGTSESATPESGPGTSTEPSEGSAPGTSTEPSEGSAPGTSTEPSEGSA |
| | | PGSPAGSPTSTEEGTSTEPSEGSAPG |

4. XTEN segments

In one embodiment, the invention provides an isolated hGH-XTEN fusion protein wherein the cumulative length of the XTEN component is greater than about 100 to about 3000 amino acid residues containing at least one polypeptide sequence segment selected from Table 3 (and Tables 8, 9, 10, 11, and 12 of Schellenberger et al. WO10/144502A2, which is incorporated herein by reference in its entirety) and wherein at least about 90%, or at least about 91%, or at least about 92%, or at least about 93%, or at least about 94%, or at least about 95%, or at least about 96%, or at least about 97%, or at least about 98% or more of the remainder of the XTEN sequence by and large contains hydrophilic amino acids and less than about 2% of the remainder of the XTEN consists of hydrophobic or aromatic amino acids, or cysteine. In some embodiments, the XTEN contains multiple segments wherein the segments are identical or different. In another embodiment, the invention provides an isolated hGH-XTEN fusion protein wherein the cumulative length of the XTEN component is greater than about 100 to about 3000 amino acid residues and comprises at least one sequence segment of at least about 100 to about 923, or at least about 100 to about 875, or at least about 100 to about 576, or at least about 100 to about 288, or at least about 100 to about 144 amino acid residues wherein the sequence segment(s) consists of at least three different types of amino acids and the sum of glycine (G), alanine (A), serine (S), threonine (T), glutamate (E) and proline (P) residues in the sequence segment(s) constitutes at least about 90%, or at least about 91%, or at least about 92%, or at least about 93%, or at least about 94%, or at least about 95%, or at least about 96%, or at least about 97%, or at least about 98%, or at least about 99% of the total amino acid sequence of the sequence segment and at least about 90%, or at least about 91%, or at least about 92%, or at least about 93%, or at least about 94%, or at least about 95%, or at least about 96%, or at least about 97%, or at least about 98% of the remainder of the XTEN sequence(s) consist of hydrophilic amino acids and less than about 2% of the remainder of the XTEN sequence(s) consists of hydrophobic or aromatic amino acids, or cysteine. In another embodiment, the invention provides an isolated hGH-XTEN fusion protein wherein the cumulative length of the XTEN component is greater than about 100 to about 3000 amino acid residues and comprises at least one sequence segment of at least about 200 to about 923, or at least about 200 to about 875, or at least about 200 to about 576, or at least about 200 to about 288 amino acid residues wherein the sequence segment(s) the sum of glycine (G), alanine (A), serine (S), threonine (T), glutamate (E) and proline (P) residues in the sequence segment(s) constitutes at least about 90%, or at least about 91%, or at least about 92%, or at least about 93%, or at least about 94%, or at least about 95%, or at least about 96%, or at least about 97%, or at least about 98%, or at least about 99% of the total amino acid sequence of the sequence segment and wherein the subsequence score of the segment is less than 12, more preferably less than 10, more preferably less than 9, more preferably less than 8, more preferably less than 7, more preferably less than 6, and most preferably less than 5, and at least about 90%, or at least about 91%, or at least about 92%, or at least about 93%, or at least about 94%, or at least about 95%, or at least about 96%, or at least about 97%, or at least about 98% of the remainder of the XTEN sequence(s) consist of hydrophilic amino acids and less than about 2% of the remainder of the XTEN sequence(s) consists of hydrophobic, aromatic or cysteine amino acids.

5. N-Terminal XTEN Expression-Enhancing Sequences In some embodiments, the invention provides a short-length XTEN sequence incorporated as the N-terminal portion of the hGH-XTEN fusion protein. The expression of the fusion protein is enhanced in a host cell transformed with a suitable expression vector comprising an optimized N-terminal leader polynucleotide sequence (that encodes the N-terminal XTEN) incorporated into the polynucleotide encoding the binding fusion protein. It has been discovered, as described in Examples 14-17 of Schellenberger et al. WO10/144502A2 (which is incorporated herein by reference in its entirety), that a host cell transformed with such an expression vector comprising an optimized N-terminal leader sequence (NTS) in the binding fusion protein gene results in greatly-enhanced expression of the fusion protein compared to the expression of a corresponding fusion protein from a polynucleotide not comprising the NTS, and obviates the need for incorporation of a non-XTEN leader sequence used to enhance expression.

In one embodiment, the invention provides hGH-XTEN fusion proteins comprising an NTS wherein the expression of the binding fusion protein from the encoding gene in a host cell is enhanced about 50%, or about 75%, or about 100%, or about 150%, or about 200%, or about 400% compared to expression of a hGH-XTEN fusion protein not comprising the N-terminal XTEN sequence (where the encoding gene lacks the NTS).

In one embodiment, the N-terminal XTEN polypeptide of the hGH-XTEN comprises a sequence that exhibits at least about 80%, more preferably at least about 90%, more preferably at least about 91%, more preferably at least about 92%, more preferably at least about 93%, more preferably at least about 94%, more preferably at least about 95%, more preferably at least about 96%, more preferably at least about 97%, more preferably at least about 98%, more preferably at least 99%, or exhibits 100% sequence identity to the amino acid sequence of AE48, AE48.1, AM48, or AM48.1, the respective amino acid sequences of which are as follows:

AE48:
(SEQ ID NO: 54)
MAEPAGSPTSTEEGTPGSGTASSSPGSSTPSGATGSPGASPGTSSTGS
AE48.1:
(SEQ ID NO: 81)
AEPAGSPTSTEEGTPGSGTASSSPGSSTPSGATGSPGASPGTSSTGS
AM48:
(SEQ ID NO: 55)
MAEPAGSPTSTEEGASPGTSSTGSPGSSTPSGATGSPGSSTPSGATGS
AM48.1:
(SEQ ID NO: 82)
AEPAGSPTSTEEGASPGTSSTGSPGSSTPSGATGSPGSSTPSGATGS.

In another embodiment, the N-terminal XTEN polypeptide of the hGH-XTEN comprises a sequence exhibiting a % identity to AE48, AM48 or AE912, as described herein, wherein the N-terminal M residue is absent (e.g., AE48.1—SEQ ID NO: 81; AM48.1—SEQ ID NO: 82; and AE912.1—SEQ ID NO: 83). In an additional embodiment, the C-terminal XTEN poly peptide of the hGH-XTEN comprises a sequence exhibiting a % identity to AE146, as described herein, (e.g., AE146—SEQ ID NO: 53; or AE146.1—SEQ ID NO: 85).

In another embodiment, the short-length N-terminal XTEN is linked to an XTEN of longer length to form the N-terminal region of the hGH-XTEN fusion protein, wherein the polynucleotide sequence encoding the short-length N-terminal XTEN confers the property of enhanced expression in the host cell, and wherein the long length of the expressed XTEN contributes to the enhanced properties of the XTEN carrier in the fusion protein, as described above. In the foregoing, the short-length XTEN is linked to any of the XTEN disclosed herein (e.g., an XTEN of Table 3) and the resulting XTEN, in turn, is linked to the N-terminal of any of the GH disclosed herein (e.g., a GH comprising the sequence of SEQ ID NO:2) as a component of the fusion protein. Alternatively, polynucleotides encoding the short-length XTEN (or its complement) is linked to polynucleotides encoding any of the XTEN (or its complement) disclosed herein and the resulting gene encoding the N-terminal XTEN, in turn, is linked to the 5′ end of polynucleotides encoding any of the GH (or to the 3′ end of its complement) disclosed herein. In some embodiments, the N-terminal XTEN polypeptide with long length exhibits at least about 80%, or at least about 90%, or at least about 91%, or at least about 92%, or at least about 93%, or at least about 94%, or at least about 95%, or at least about 96%, or at least about 97%, or at least about 98%, or at least 99%, or exhibits 100% sequence identity to an amino acid sequence selected from the group consisting of the sequences AE624, AE911, AE912, and AM923.

In any of the foregoing N-terminal XTEN embodiments described above, the N-terminal XTEN can have from about one to about six additional amino acid residues, preferably selected from GESTPA, to accommodate the restriction endonuclease restriction sites that would be employed to join the nucleotides encoding the N-terminal XTEN to the gene encoding the targeting moiety of the fusion protein. The methods for the generation of the N-terminal sequences and incorporation into the fusion proteins of the invention are described more fully in the Examples.

6. Net Charge

In other embodiments, the XTEN polypeptides have an unstructured characteristic imparted by incorporation of amino acid residues with a net charge and/or reducing the proportion of hydrophobic amino acids in the XTEN sequence. The overall net charge and net charge density is controlled by modifying the content of charged amino acids in the XTEN sequences. In some embodiments, the net charge density of the XTEN of the compositions may be above +0.1 or below −0.1 charges/residue. In other embodiments, the net charge of a XTEN can be about 0%, about 1%, about 2%, about 3%, about 4%, about 5%, about 6%, about 7%, about 8%, about 9%, about 10% about 11%, about 12%, about 13%, about 14%, about 15%, about 16%, about 17%, about 18%, about 19%, or about 20% or more.

Since most tissues and surfaces in a human or animal have a net negative charge, in some embodiments, the XTEN sequences are designed to have a net negative charge to minimize non-specific interactions between the XTEN containing compositions and various surfaces such as blood vessels, healthy tissues, or various receptors. Not to be bound by a particular theory, the XTEN can adopt open conformations due to electrostatic repulsion between individual amino acids of the XTEN polypeptide that individually carry a net negative charge and that are distributed across the sequence of the XTEN polypeptide. Such a distribution of net negative charge in the extended sequence lengths of XTEN can lead to an unstructured conformation that, in turn, can result in an effective increase in hydrodynamic radius. In preferred embodiments, the negative charge is conferred by incorporation of glutamic acid residues. Accordingly, in one embodiment the invention provides XTEN in which the XTEN sequences contain about 8, 10, 15, 20, 25, or even about 30% glutamic acid. Generally, the glutamic residues would be spaced uniformly across the XTEN sequence. In some cases, the XTEN can contain about 10-80, or about 15-60, or about 20-50 glutamic residues per 20 kD of XTEN that can result in an XTEN with charged residues that would have very similar pKa, which can increase the charge homogeneity of the product and sharpen its isoelectric point, enhancing the physicochemical properties of the resulting hGH-XTEN fusion protein for, example, simplifying purification procedures.

The XTEN of the compositions of the present invention generally have no or a low content of positively charged amino acids. In some embodiments the XTEN may have less than about 10% amino acid residues with a positive charge, or less than about 7%, or less than about 5%, or less than about 2%, or less than about 1% amino acid residues with a positive charge. However, the invention contemplates constructs where a limited number of amino acids with a positive charge, such as lysine, are incorporated into XTEN to permit conjugation between the epsilon amine of the lysine and a reactive group on a peptide, a linker bridge, or a reactive group on a drug or small molecule to be conjugated to the XTEN backbone. In one embodiment of the foregoing, the XTEN has between about 1 to about 100 lysine residues, or about 1 to about 70 lysine residues, or about 1 to about 50 lysine residues, or about 1 to about 30 lysine residues, or about 1 to about 20 lysine residues, or about 1 to about 10 lysine residues, or about 1 to about 5 lysine residues, or alternatively only a single lysine residue. Using the foregoing lysine-containing XTEN, fusion proteins are constructed that comprises XTEN, a growth hormone, plus a chemotherapeutic agent useful in the treatment of growth-related diseases or disorders, wherein the maximum number of molecules of the agent incorporated into the XTEN component is determined by the numbers of lysines or other amino acids with reactive side chains (e.g., cysteine) incorporated into the XTEN.

In some embodiments, the XTEN sequence comprises charged residues separated by other residues such as serine or glycine, which leads to better expression or purification behavior. Based on the net charge, some XTENs have an isoelectric point (pI) of 1.0, 1.5, 2.0, 2.5, 3.0, 3.5, 4.0, 4.5, 5.0, 5.5, 6.0, or even 6.5. In preferred embodiments, the XTEN will have an isoelectric point between 1.5 and 4.5. In these embodiments, the XTEN incorporated into the hGH-XTEN fusion protein compositions of the present invention carry a net negative charge under physiologic conditions that contribute to the unstructured conformation and reduced binding of the XTEN component to mammalian proteins and tissues.

As hydrophobic amino acids impart structure to a polypeptide, the invention provides that the content of hydrophobic amino acids in the XTEN will typically be less than 5%, or less than 2%, or less than 1% hydrophobic amino acid content. In one embodiment, the amino acid content of methionine and tryptophan in the XTEN component of a hGH-XTEN fusion protein is typically less than 5%, or less than 2%, and most preferably less than 1%. In another embodiment, the XTEN will have a sequence that has less than 10% amino acid residues with a positive charge, or less than about 7%, or less that about 5%, or less than about 2% amino acid residues with a positive charge, the sum of methionine and tryptophan residues will be less than 2%, and the sum of asparagine and glutamine residues will be less than 10% of the total XTEN sequence.

7. Low Immunogenicity

In another aspect, the invention provides compositions in which the XTEN sequences have a low degree of immunogenicity or are substantially non-immunogenic. Several factors can contribute to the low immunogenicity of XTEN, e.g., the non-repetitive sequence, the unstructured conformation, the high degree of solubility, the low degree or lack of self-aggregation, the low degree or lack of proteolytic sites within the sequence, and the low degree or lack of epitopes in the XTEN sequence.

Conformational epitopes are formed by regions of the protein surface that are composed of multiple discontinuous amino acid sequences of the protein antigen. The precise folding of the protein brings these sequences into a well-defined, stable spatial configurations, or epitopes, that can be recognized as “foreign” by the host humoral immune system, resulting in the production of antibodies to the protein or the activation of a cell-mediated immune response. In the latter case, the immune response to a protein in an individual is heavily influenced by T-cell epitope recognition that is a function of the peptide binding specificity of that individual's HLA-DR allotype. Engagement of a MHC Class II peptide complex by a cognate T-cell receptor on the surface of the T-cell, together with the cross-binding of certain other co-receptors such as the CD4 molecule, can induce an activated state within the T-cell. Activation leads to the release of cytokines further activating other lymphocytes such as B cells to produce antibodies or activating T killer cells as a full cellular immune response.

The ability of a peptide to bind a given MHC Class II molecule for presentation on the surface of an APC (antigen presenting cell) is dependent on a number of factors; most notably its primary sequence. In one embodiment, a lower degree of immunogenicity is achieved by designing XTEN sequences that resist antigen processing in antigen presenting cells, and/or choosing sequences that do not bind MHC receptors well. The invention provides hGH-XTEN fusion proteins with substantially non-repetitive XTEN polypeptides designed to reduce binding with MHC II receptors, as well as avoiding formation of epitopes for T-cell receptor or antibody binding, resulting in a low degree of immunogenicity. Avoidance of immunogenicity is, in part, a direct result of the conformational flexibility of XTEN sequences; i.e., the lack of secondary structure due to the selection and order of amino acid residues. For example, of particular interest are sequences having a low tendency to adapt compactly folded conformations in aqueous solution or under physiologic conditions that could result in conformational epitopes. The administration of fusion proteins comprising XTEN, using conventional therapeutic practices and dosing, would generally not result in the formation of neutralizing antibodies to the XTEN sequence, and also reduce the immunogenicity of the GH fusion partner in the hGH-XTEN compositions.

In one embodiment, the XTEN sequences utilized in the subject fusion proteins can be substantially free of epitopes recognized by human T cells. The elimination of such epitopes for the purpose of generating less immunogenic proteins has been disclosed previously; see for example WO 98/52976, WO 02/079232, and WO 00/3317 which are incorporated by reference herein. Assays for human T cell epitopes have been described (Stickler, M., et al. (2003) J Immunol Methods, 281: 95-108). Of particular interest are peptide sequences that can be oligomerized without generating T cell epitopes or non-human sequences. This is achieved by testing direct repeats of these sequences for the presence of T-cell epitopes and for the occurrence of 6 to 15-mer and, in particular, 9-mer sequences that are not human, and then altering the design of the XTEN sequence to eliminate or disrupt the epitope sequence. In some embodiments, the XTEN sequences are substantially non-immunogenic by the restriction of the numbers of epitopes of the XTEN predicted to bind MHC receptors. With a reduction in the numbers of epitopes capable of binding to MHC receptors, there is a concomitant reduction in the potential for T cell activation as well as T cell helper function, reduced B cell activation or upregulation and reduced antibody production. The low degree of predicted T-cell epitopes can be determined by epitope prediction algorithms such as, e.g., TEPITOPE (Sturniolo, T., et al. (1999) Nat Biotechnol, 17: 555-61), as shown in Example 45. The TEPITOPE score of a given peptide frame within a protein is the log of the Kd (dissociation constant, affinity, off-rate) of the binding of that peptide frame to multiple of the most common human MHC alleles, as disclosed in Sturniolo, T. et al. (1999) Nature Biotechnology 17:555). The score ranges over at least 20 logs, from about 10 to about −10 (corresponding to binding constraints of 10e10 Kd to 10e-10 Kd), and can be reduced by avoiding hydrophobic amino acids that serve as anchor residues during peptide display on MHC, such as M, I, L, V, F. In some embodiments, an XTEN component incorporated into a hGH-XTEN does not have a predicted T-cell epitope at a TEPITOPE score of about −5 or greater, or −6 or greater, or −7 or greater, or −8 or greater, or at a TEPITOPE score of −9 or greater. As used herein, a score of “−9 or greater” would encompass TEPITOPE scores of 10 to −9, inclusive, but would not encompass a score of −10, as −10 is less than −9.

In another embodiment, the inventive XTEN sequences, including those incorporated into the subject hGH-XTEN fusion proteins, are rendered substantially non-immunogenic by the restriction of known proteolytic sites from the sequence of the XTEN, reducing the processing of XTEN into small peptides that can bind to MHC II receptors. In another embodiment, the XTEN sequence is rendered substantially non-immunogenic by the use a sequence that is substantially devoid of secondary structure, conferring resistance to many proteases due to the high entropy of the structure. Accordingly, the reduced TEPITOPE score and elimination of known proteolytic sites from the XTEN render the XTEN compositions, including the XTEN of the hGH-XTEN fusion protein compositions, substantially unable to be bound by mammalian receptors, including those of the immune system. In one embodiment, an XTEN of a hGH-XTEN fusion protein can have >100 nM Kd binding to a mammalian receptor, or greater than 500 nM Kd, or greater than 1 μM Kd towards a mammalian cell surface or circulating polypeptide receptor.

Additionally, the non-repetitive sequence and corresponding lack of epitopes of XTEN limit the ability of B cells to bind to or be activated by XTEN. A repetitive sequence is recognized and can form multivalent contacts with even a few B cells and, as a consequence of the cross-linking of multiple T-cell independent receptors, can stimulate B cell proliferation and antibody production. In contrast, while a XTEN can make contacts with many different B cells over its extended sequence, each individual B cell may only make one or a small number of contacts with an individual XTEN due to the lack of repetitiveness of the sequence. Not being to be bound by any theory, XTENs typically have a much lower tendency to stimulate proliferation of B cells and thus an immune response. In one embodiment, the hGH-XTEN have reduced immunogenicity as compared to the corresponding GH that is not fused. In one embodiment, the administration of up to three parenteral doses of a hGH-XTEN to a mammal result in detectable anti-hGH-XTEN IgG at a serum dilution of 1:100 but not at a dilution of 1:1000. In another embodiment, the administration of up to three parenteral doses of a hGH-XTEN to a mammal result in detectable anti-GH IgG at a serum dilution of 1:100 but not at a dilution of 1:1000. In another embodiment, the administration of up to three parenteral doses of a hGH-XTEN to a mammal result in detectable anti-XTEN IgG at a serum dilution of 1:100 but not at a dilution of 1:1000. In the foregoing embodiments, the mammal can be a mouse, a rat, a rabbit, or a cynomolgus monkey.

An additional feature of XTENs with non-repetitive sequences relative to sequences with a high degree of repetitiveness is non-repetitive XTENs form weaker contacts with antibodies. Antibodies are multivalent molecules. For instance, IgGs have two identical binding sites and IgMs contain 10 identical binding sites. Thus antibodies against repetitive sequences can form multivalent contacts with such repetitive sequences with high avidity, which can affect the potency and/or elimination of such repetitive sequences. In contrast, antibodies against non-repetitive XTENs may yield monovalent interactions, resulting in less likelihood of immune clearance such that the hGH-XTEN compositions can remain in circulation for an increased period of time.

8. Increased Hydrodynamic Radius

In another aspect, the present invention provides XTEN in which the XTEN polypeptides have a high hydrodynamic radius that confers a corresponding increased Apparent Molecular Weight to the hGH-XTEN fusion protein incorporating the XTEN. As detailed in Example 37, the linking of XTEN to GH sequences results in hGH-XTEN compositions that can have increased hydrodynamic radii, increased Apparent Molecular Weight, and increased Apparent Molecular Weight Factor compared to a GH not linked to an XTEN. For example, in therapeutic applications in which prolonged half-life is desired, compositions in which a XTEN with a high hydrodynamic radius is incorporated into a fusion protein comprising one or more GH can effectively enlarge the hydrodynamic radius of the composition beyond the glomerular pore size of approximately 3-5 nm (corresponding to an apparent molecular weight of about 70 kDA) (Caliceti. 2003. Pharmacokinetic and biodistribution properties of poly(ethylene glycol)-protein conjugates. Adv Drug Deliv Rev 55:1261-1277), resulting in reduced renal clearance of circulating proteins. The hydrodynamic radius of a protein is determined by its molecular weight as well as by its structure, including shape or compactness. Not to be bound by a particular theory, the XTEN can adopt open conformations due to electrostatic repulsion between individual charges of the peptide or the inherent flexibility imparted by the particular amino acids in the sequence that lack potential to confer secondary structure. The open, extended and unstructured conformation of the XTEN polypeptide can have a greater proportional hydrodynamic radius compared to polypeptides of a comparable sequence length and/or molecular weight that have secondary and/or tertiary structure, such as typical globular proteins. Methods for determining the hydrodynamic radius are well known in the art, such as by the use of size exclusion chromatography (SEC), as described in U.S. Pat. Nos. 6,406,632 and 7,294,513. As the results of Example 37 of Schellenberger et al. WO10/144502A2 (which is incorporated herein by reference in its entirety) demonstrate, the addition of increasing lengths of XTEN results in proportional increases in the parameters of hydrodynamic radius, Apparent Molecular Weight, and Apparent Molecular Weight Factor, permitting the tailoring of hGH-XTEN to desired characteristic cut-off Apparent Molecular Weights or hydrodynamic radii. Accordingly, in certain embodiments, the hGH-XTEN fusion protein can be configured with an XTEN such that the fusion protein can have a hydrodynamic radius of at least about 5 nm, or at least about 8 nm, or at least about 10 nm, or 12 nm, or at least about 15 nm. In the foregoing embodiments, the large hydrodynamic radius conferred by the XTEN in an hGH-XTEN fusion protein can lead to reduced renal clearance of the resulting fusion protein, leading to a corresponding increase in terminal half-life, an increase in mean residence time, and/or a decrease in renal clearance rate.

In another embodiment, an XTEN of a chosen length and sequence can be selectively incorporated into a hGH-XTEN to create a fusion protein that have, under physiologic conditions, an Apparent Molecular Weight of at least about 150 kDa, or at least about 300 kDa, or at least about 400 kDa, or at least about 500 kDA, or at least about 600 kDa, or at least about 700 kDA, or at least about 800 kDa, or at least about 900 kDa, or at least about 1000 kDa, or at least about 1200 kDa, or at least about 1500 kDa, or at least about 1800 kDa, or at least about 2000 kDa, or at least about 2300 kDa or more. In another embodiment, an XTEN of a chosen length and sequence can be selectively linked to a GH to result in a hGH-XTEN fusion protein that has, under physiologic conditions, an Apparent Molecular Weight Factor of at least three, alternatively of at least four, alternatively of at least five, alternatively of at least six, alternatively of at least eight, alternatively of at least 10, alternatively of at least 15, or an Apparent Molecular Weight Factor of at least 20 or greater. In another embodiment, the hGH-XTEN fusion protein has, under physiologic conditions, an Apparent Molecular Weight Factor that is about 4 to about 20, or is about 6 to about 15, or is about 8 to about 12, or is about 9 to about 10 relative to the actual molecular weight of the fusion protein.

V). hGH-XTEN Structural Configurations and Properties

The human growth hormone (GH) of the subject compositions are not limited to native, full-length polypeptides, but also include recombinant versions as well as biologically and/or pharmacologically active variants or fragments thereof. For example, it will be appreciated that various amino acid deletions, insertions and substitutions can be made in the GH to create variants without departing from the spirit of the invention with respect to the biological activity or pharmacologic properties of the GH. Examples of conservative substitutions for amino acids in polypeptide sequences are shown in Table 4. However, in embodiments of the hGH-XTEN in which the sequence identity of the GH is less than 100% compared to a specific sequence disclosed herein, the invention contemplates substitution of any of the other 19 natural L-amino acids for a given amino acid residue of the given GH, which may be at any position within the sequence of the GH, including adjacent amino acid residues. If any one substitution results in an undesirable change in biological activity, then one of the alternative amino acids can be employed and the construct evaluated by the methods described herein, or using any of the techniques and guidelines for conservative and non-conservative mutations set forth, for instance, in U.S. Pat. No. 5,364,934, the contents of which is incorporated by reference in its entirety, or using methods generally known in the art. In addition, variants can include, for instance, polypeptides wherein one or more amino acid residues are added or deleted at the N— or C-terminus of the full-length native amino acid sequence of a GH that retains some if not all of the biological activity of the native peptide.

TABLE 4
Exemplary conservative amino acid substitutions

| Original Residue | Exemplary Substitutions |
|---|---|
| Ala (A) | val; leu; ile |
| Arg (R) | lys; gin; asn |
| Asn (N) | gin; his; Iys; arg |
| Asp (D) | glu |
| Cys (C) | ser |
| Gln (Q) | asn |
| Glu (E) | asp |
| Gly (G) | pro |
| His (H) | asn: gin: Iys: arg |
| xIle (I) | leu; val; met; ala; phe: norleucine |
| Leu (L) | norleucine: ile: val; met; ala: phe |
| Lys (K) | arg: gin: asn |
| Met (M) | leu; phe; ile |
| Phe (F) | leu: val: ile; ala |
| Pro (P) | gly |
| Ser (S) | thr |
| Thr (T) | ser |
| Trp (W) | tyr |
| Tyr(Y) | trp: phe: thr: ser |
| Val (V) | ile; leu; met; phe; ala; norleucine |

(a) Fusion Protein Configurations

The invention provides fusion protein compositions with the GH and XTEN components linked in specific N— to C-terminus configurations. In some embodiments, one or more GHs are linked to one or more XTENs, either at the N-terminus or at the C-terminus, with or without a spacer, to form a block copolymer, and the sequential arrangement of the GHs and the XTENs in the fusion protein are the same as the configuration known in the block copolymer chemistry. When there is more than one GH, XTEN, or spacer, each of the GH, the XTEN, or the spacer have the same or different sequences, and the GHs and/or XTENs are linked either continuously or alternately (regular or irregular). Thus, in all of the formulae provided herein, when there is more than one GH, XTEN, or spacer, each of the GH, XTEN, and spacer are the same or different. In some embodiments, the fusion protein is a monomeric fusion protein with a GH linked to one XTEN polypeptide. In other embodiments, the fusion protein is a monomeric fusion protein with a GH linked to two or more XTEN polypeptides. In still other embodiments, the fusion protein is a monomeric fusion protein with two or more GH linked to one XTEN polypeptide. In still other embodiments, the fusion protein is a monomeric fusion protein with two or more GH linked to two or more XTEN polypeptide. Table 5 provides non-limiting examples of configurations that are encompassed by the invention; numerous other variations will be apparent to the ordinarily skilled artisan, including the incorporation the spacer and cleavage sequences disclosed herein or known in the art.

TABLE 5
hGH-XTEN configurations

| Components* | Configuration** |
|---|---|
| Single GH; Single XTEN | GH-XTEN |
| | XTEN-GH |
| Single GH; Multiple XTEN | XTEN-GH-XTEN |
| | GH-XTEN-XTEN |
| | XTEN-XTEN-GH |
| | XTEN-GH-XTEN-XTEN |
| | XTEN-XTEN-GH-XTEN |
| | XTEN-XTEN-GH-XTEN |
| Multiple GH, Single XTEN | GH-XTEN-GH |
| | XTEN-GH-GH |
| | GH-GH-XTEN |
| | GH-XTEN-GH-GH |
| Multiple GH; Multiple XTEN | GH-XTEN-GH-XTEN |
| | XTEN-GH-XTEN-GH |
| | XTEN-XTEN-GH-XTEN-GH |
| | XTEN-XTEN-GH-GH |
| | GH-XTEN-XTEN-GH |
| | GH-GH-XTEN-XTEN |
| | GH-GH-XTEN-XTEN-GH |
| | GH-XTEN-GH-XTEN-GH |

*Characterized as single for 1 component or multiple for 2 or more of that component
**Reflects N- to C-terminus configuration of the growth factor and XTEN components

The invention contemplates fusion proteins compositions that are in a configuration shown in Table 5 and that retain at least a portion of the biological activity of the corresponding GH not linked to the XTEN. In other embodiments, the GH component either becomes biologically active or has an increase in activity upon its release from the XTEN by cleavage of an optional cleavage sequence incorporated within spacer sequences into the hGH-XTEN, described more fully below.

In one embodiment of the hGH-XTEN composition, the invention provides a fusion protein of formula I:

(XTEN)ₓ-GH-(XTEN)_{y} I

wherein independently for each occurrence, GH is a human growth hormone; x is either 0 or 1 and y is either 0 or 1 wherein x+y≧1; and XTEN is an extended recombinant polypeptide.

In another embodiment of the hGH-XTEN composition, the invention provides a fusion protein of formula II:

(XTEN)ₓ-(GH)-(S)_{y}-(XTEN)_{y} II

wherein independently for each occurrence, GH is a human growth hormone; S is a spacer sequence having between 1 to about 50 amino acid residues that can optionally include a cleavage sequence; x is either 0 or 1 and y is either 0 or 1 wherein x+y≧1; and XTEN is an extended recombinant polypeptide.

In another embodiment, the invention provides an isolated fusion protein, wherein the fusion protein is of formula III:

(GH)—(S)ₓ-(XTEN)-(S)_{y}-(GH)-(S)_{z}—(XTEN)_{z} III

wherein independently for each occurrence, GH is a human growth hormone; S is a spacer sequence having between 1 to about 50 amino acid residues that can optionally include a cleavage sequence; x is either 0 or 1; y is either 0 or 1; z is either 0 or 1; and XTEN is an extended recombinant polypeptide.

In another embodiment, the invention provides an isolated fusion protein, wherein the fusion protein is of formula IV:

(XTEN)ₓ-(S)_{y}-(GH)-(S)_{z}-(XTEN)-(GH) IV

wherein independently for each occurrence, GH is a human growth hormone; S is a spacer sequence having between 1 to about 50 amino acid residues that can optionally include a cleavage sequence; x is either 0 or 1; y is either 0 or 1; z is either 0 or 1; and XTEN is an extended recombinant polypeptide.

In another embodiment, the invention provides an isolated fusion protein, wherein the fusion protein is of formula V:

(GH)ₓ-(S)ₓ-(GH)-(S)_{y}-(XTEN) V

wherein independently for each occurrence, GH is a growth hormone; S is a spacer sequence having between 1 to about 50 amino acid residues that can optionally include a cleavage sequence; x is either 0 or 1; y is either 0 or 1; and XTEN is an extended recombinant polypeptide.

In another embodiment, the invention provides an isolated fusion protein, wherein the fusion protein is of formula VI:

(XTEN)-(S)ₓ-(GH)-(S)-(GH) VI

wherein independently for each occurrence, GH is a growth hormone; S is a spacer sequence having between 1 to about 50 amino acid residues that can optionally include a cleavage sequence; x is either 0 or 1; y is either 0 or 1; and XTEN is an extended recombinant polypeptide.

In another embodiment, the invention provides an isolated fusion protein, wherein the fusion protein is of formula VII:

(XTEN)-(S)ₓ-(GH)-(S)_{y}-(GH)—(XTEN) VII

wherein independently for each occurrence, GH is a growth hormone; S is a spacer sequence having between 1 to about 50 amino acid residues that can optionally include a cleavage sequence; x is either 0 or 1; y is either 0 or 1; and XTEN is an extended recombinant polypeptide.

In another embodiment, the invention provides an isolated fusion protein, wherein the fusion protein is of formula VIII:

((S)ₘ-(GH)ₓ-(S)ₓ-(XTEN)_{y}-(S)ₒ)ₜ VIII

wherein t is an integer that is greater than 0 (1, 2, 3, etc.); independently each of m, n, o, x, and y is an integer (0, 1, 2, 3, etc.), GH is a growth hormone; S is an spacer, optionally comprising a cleavage site; and XTEN is an extended recombinant polypeptide, with the proviso that: (1) x+y>1, (2) when t=1, x>0 and y>0, (3) when there is more than one GH, S, or XTEN, each GH, XTEN, or S are the same or are independently different; and (4) when t>1, each m, n, o, x, or y within each subunit are the same or are independently different.

In another embodiment, the invention provides an isolated fusion protein, wherein the fusion protein is of formula IX:

(XTEN)ₓ-(S)ₓ-(GH)-(S)_{y}-(XTEN)_{y} IX

wherein independently for each occurrence, GH is a human growth hormone; S is a spacer sequence having between 1 to about 50 amino acid residues that can optionally include a cleavage sequence; x is either 0 or 1 and y is either 0 or 1 wherein x+y≧1; and XTEN is an extended recombinant polypeptide.

In some embodiments, administration of a therapeutically effective amount of a fusion protein of an embodiment of formulas I-VIII to a subject in need thereof results in a gain in time of at least two-fold, or at least three-fold, or at least four-fold, or at least five-fold, or at least 10-fold, or at least 20-fold, or at least 40-fold, or at least 100-fold or more spent within a therapeutic window for the fusion protein compared to the corresponding GH not linked to the XTEN and administered at a comparable amount administered to a subject. In other embodiments, administration of a therapeutically effective dose of a fusion protein of an embodiment of formulas I-VIII to a subject in need thereof can result in a gain in time between consecutive doses necessary to maintain a therapeutically effective dose regimen of at least 48 h, or at least 72 h, or at least about 96 h, or at least about 120 h, or at least about 7 days, or at least about 14 days, or at least about 21 days, or at least about 28 days, or at least about monthly between consecutive doses compared to a dose schedule for GH not linked to required to maintain a therapeutically effective dose regimen.

Any spacer sequence group is optional in the fusion proteins encompassed by the invention. The spacer is provided to enhance expression of the fusion protein from a host cell or to decrease steric hindrance such that the GH component may assume its desired tertiary structure and/or interact appropriately with its target receptor. For spacers and methods of identifying desirable spacers, see, for example, George, et al. (2003) Protein Engineering 15:871-879, specifically incorporated by reference herein. In one embodiment, the spacer comprises one or more peptide sequences that are between 1-50 amino acid residues in length, or about 1-25 residues, or about 1-10 residues in length. Spacer sequences, exclusive of cleavage sites, can comprise any of the 20 natural L amino acids, and will preferably comprise hydrophilic amino acids that are sterically unhindered that can include, but not be limited to, glycine (G), alanine (A), serine (S), threonine (T), glutamate (E) and proline (P). In some cases, the spacer can be polyglycines or polyalanines, or is predominately a mixture of combinations of glycine and alanine residues. The spacer polypeptide exclusive of a cleavage sequence is largely to substantially devoid of secondary structure; e.g., less than about 10%, or less than about 5% as determined by the Chou-Fasman and/or GOR algorithms. In one embodiment, one or both spacer sequences in a hGH-XTEN fusion protein composition each further contains a cleavage sequence, which are identical or different, wherein the cleavage sequence may be acted on by a protease to release the GH from the fusion protein.

In some embodiments, the incorporation of the cleavage sequence into the hGH-XTEN is designed to permit release of a GH that becomes active or more active upon its release from the XTEN. The cleavage sequences are located sufficiently close to the GH sequences, generally within 18, or within 12, or within 6, or within 2 amino acids of the GH sequence terminus, such that any remaining residues attached to the GH after cleavage do not appreciably interfere with the activity (e.g., such as binding to a receptor) of the GH, yet provide sufficient access to the protease to be able to effect cleavage of the cleavage sequence. In some embodiments, the cleavage site is a sequence that can be cleaved by a protease endogenous to the mammalian subject such that the hGH-XTEN can be cleaved after administration to a subject. In such cases, the hGH-XTEN can serve as a prodrug or a circulating depot for the GH. Examples of cleavage sites contemplated by the invention include, but are not limited to, a polypeptide sequence cleavable by a mammalian endogenous protease selected from FXIa, FXIIa, kallikrein, FVIIa, FIXa, FXa, FIIa (thrombin), Elastase-2, granzyme B, MMP-12, MMP-13, MMP-17 or MMP-20, or by non-mammalian proteases such as TEV, enterokinase, PreScission™ protease (rhinovirus 3C protease), and sortase A. Sequences known to be cleaved by the foregoing proteases and others are known in the art. Exemplary cleavage sequences and cut sites within the sequences are presented in Table 6, as well as sequence variants thereof. For example, thrombin (activated clotting factor II) acts on the sequence LTPRSLLV (SEQ ID NO: 56) [Rawlings N. D., et al. (2008) Nucleic Acids Res., 36: D320], which would be cut after the arginine at position 4 in the sequence. Active FIIa is produced by cleavage of FII by FXa in the presence of phospholipids and calcium and is down stream from factor IX in the coagulation pathway. Once activated its natural role in coagulation is to cleave fibrinogen, which then in turn, begins clot formation. FIIa activity is tightly controlled and only occurs when coagulation is necessary for proper hemostasis. However, as coagulation is an on-going process in mammals, by incorporation of the LTPRSLLV sequence (SEQ ID NO: 57) into the hGH-XTEN between the GH and the XTEN, the XTEN domain would be removed from the adjoining GH concurrent with activation of either the extrinsic or intrinsic coagulation pathways when coagulation is required physiologically, thereby releasing GH over time. Similarly, incorporation of other sequences into hGH-XTEN that are acted upon by endogenous proteases would provide for sustained release of GH that, in certain embodiments, provide a higher degree of activity for the GH from the “prodrug” form of the hGH-XTEN.

In some embodiments, only the two or three amino acids flanking both sides of the cut site (four to six amino acids total) are incorporated into the cleavage sequence. In other embodiments, the known cleavage sequence have one or more deletions or insertions or one or two or three amino acid substitutions for any one or two or three amino acids in the known sequence, wherein the deletions, insertions or substitutions result in reduced or enhanced susceptibility but not an absence of susceptibility to the protease, resulting in an ability to tailor the rate of release of the GH from the XTEN. Exemplary substitutions are shown in Table 6.

TABLE 6
Protease Cleavage Sequences

| | Exemplary | SEQ | | |
|---|---|---|---|---|
| Protease Acting | Cleavage | ID | | SEQ |
| Upon Sequence | Sequence | NO: | Minimal Cut Site* | ID NO: |
| FXIa | KLTR↓VVGG | 58 | KD/FL/T/R↓VA/VE/GT/GV | |
| FXIIa | TMTR↓IVGG | 59 | NA | |
| Kallikrein | SPFR↓STGG | 60 | -/-/FL/RY↓SR/RT/-/- | |
| FVIIa | LQVR↓IVGG | 61 | NA | |
| FIXa | PLGR↓IVGG | 62 | -/-/G/R↓-/-/-/- | |
| FXa | IEGR↓TVGG | 63 | IA/E/GFP/R↓STI/VFS/-/G | |
| FIIa (thrombin) | LTPR↓SLLV | 64 | -/-/PLA/R↓SAG/-/-/- | |
| Elastase-2 | LGPV↓SGVP | 65 | -/-/-/VIAT↓-/-/-/- | |
| Granzyme-B | VAGD↓SLEE | 66 | V/-/-/D↓-/-/-/- | |
| MMP-12 | GPAG↓LGGA | 67 | G/PA/-/G↓L/-/G/- | 68 |
| MMP-13 | GPAG↓LRGA | 69 | G/P/-/G↓L/-/GA/- | 70 |
| MMP-17 | APLG↓LRLR | 71 | -/PS/-/-↓LQ/-/LT/- | |
| MMP-20 | PALP↓LVAQ | 72 | NA | |
| TEV | ENLYFQ↓G | 73 | ENLYFQ↓G/S | 74 |
| Enterokinase | DDDK↓IVGG | 75 | DDDK↓IVGG | 76 |
| Protease 3C | LEVLFQ↓GP | 77 | LEVLFQ↓GP | 78 |
| (PreScission ™) | | | | |
| Sortase A | LPKT↓GSES | 79 | L/P/KEAD/T↓G/-/EKS/S | 80 |

↓indicates cleavage site
NA: not applicable
*the listing of multiple amino acids before, between, or after a slash indicate alternative amino acids that can be substituted at the position;
“-” indicates that any amino acid may be substituted for the corresponding amino acid indicated in the middle column

In one embodiment, a GH incorporated into a hGH-XTEN fusion protein has a sequence that exhibits at least about 80% sequence identity to a sequence shown as SEQ ID NO: 2, alternatively at least about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%, or about 100% sequence identity as compared with the sequence of SEQ ID NO: 2. The GH of the foregoing embodiment can be evaluated for activity using assays or measured or determined parameters as described herein, and those sequences that retain at least about 40%, or about 50%, or about 55%, or about 60%, or about 70%, or about 80%, or about 90%, or about 95% or more activity compared to the corresponding native GH sequence would be considered suitable for inclusion in the subject hGH-XTEN. The GH found to retain a suitable level of activity can be linked to one or more XTEN polypeptides described hereinabove. In one embodiment, a GH found to retain a suitable level of activity can be linked to one or more XTEN polypeptides having at least about 80% sequence identity to a sequence from Table 3, alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared with a sequence of Table 3, resulting in a chimeric fusion protein.

Non-limiting examples of sequences of fusion proteins containing a single GH linked to a single XTEN are presented in Table 35 of Schellenberger et al. WO10/144,502A2, which is incorporated herein by reference in its entirety. In one embodiment, a hGH-XTEN composition would comprise a fusion protein having at least about 80% sequence identity to a hGH-XTEN from Table 35 of Schellenberger et al. WO10/144,502A2 (which is incorporated herein by reference in its entirety), alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared with a hGH-XTEN from Table 35 of Schellenberger et al. WO10/144502A2, which is incorporated herein by reference in its entirety. Non-limiting examples of sequences of fusion proteins containing two molecules of XTEN linked to one or more GH are presented in Table 36 of Schellenberger et al. WO10/144,502A2 (which is incorporated herein by reference in its entirety), but the invention also contemplates substitution of other GH with sequences exhibiting at least about 90% sequence identity to the sequence of SEQ ID NO: 2 linked to one or two XTEN, which may be the same or different, exhibiting at least about 90% sequence identity selected from Table 3. In the foregoing fusion proteins hereinabove described in this paragraph, the hGH-XTEN fusion protein can further comprise a cleavage sequence from Table 6; the cleavage sequence being located between the GH and the XTEN or between adjacent GH (if more than one GH is included in the hGH-XTEN). In some cases, the hGH-XTEN comprising the cleavage sequences will also have one or more spacer sequence amino acids between the GH and the cleavage sequence or the XTEN and the cleavage sequence to facilitate access of the protease; the spacer amino acids comprising any natural amino acid, including glycine and alanine as preferred amino acids. Non-limiting examples of hGH-XTEN comprising GH, XTEN, cleavage sequence(s) and spacer amino acids are presented in Table 37 of Schellenberger et al. WO10/144,502A2, which is incorporated herein by reference in its entirety. However, the invention also contemplates substitution of any GH sequence exhibiting at least about 90% sequence identity to the sequence of SEQ ID NO: 2 for a GH sequence of Table 37, substitution of any XTEN sequence of Table 3 for an XTEN sequence of Table 37, and substitution of any cleavage sequence of Table 6 for a cleavage sequence of Table 37 of Schellenberger et al. WO10/144502A2, which is incorporated herein by reference in its entirety.

### VI). Uses of the Compositions of the Present Invention

In one aspect, the invention provides a method for achieving a beneficial effect in a disease, disorder or condition mediated by GH including, but not limited to growth hormone deficiency in a human patient. In another aspect, the invention provides a method for achieving a beneficial effect in a disease, disorder or condition mediated by GH including, but not limited to growth hormone deficiency in adults (including adults who experienced a growth hormone-related disorder as children). The beneficial effect includes, without limitation, treating, mediating, or ameliorating a GH-related disease, deficiency, disorder or condition. The present invention addresses disadvantages and/or limitations of GH that have a relatively short terminal half-life and/or a narrow therapeutic window.

“Growth hormone deficiency” or “GHD” as used herein refers to a disease, deficiency, disorder or condition in a human patient that would benefit from treatment with growth hormone. GHD includes disorders that are classified based on the source of the GH deficiency (e.g., pituitary GHD, hypothalamic GHD, functional GHD, and idiopathic GHD). Pituitary or “classic” GHD is the incapacity of the pituitary to produce growth hormone. “Hypothalamic GHD” is the failure of the hypothalamus to produce and/or transmit the neuroendocrine messaging hormone, growth hormone releasing hormone (GHRH), which directs a properly functioning pituitary to produce GH; “functional GHD” is the failure of other hormone and of metabolic functions related to the failure of the pituitary to produce, uptake, and/or utilize GH.

In one embodiment, the human patient having a GHD is an adult. GHD includes “adult growth hormone deficiency” or “AGHD”, which may be classified based on the stage of life the GH deficiency became manifest. For example, an adult may have AGHD that is a continuation of childhood onset GHD (including child-onset GHD and child-onset idiopathic GHD), which began in infancy or childhood. The causes of childhood-onset AGHD include, without limitation, developmental defects in or near the pituitary gland; genetic problems with the production of GH; Prader-Willi syndrome; Turner's syndrome; midline facial defects; and damage to the pituitary gland or the surrounding area due to tumors, infection, radiation treatment, or severe head injury. Adults who survived brain tumors as children may be at risk of developing GHD from the effects of surgery, cranial radiation or chemotherapy.

AGHD can develop in an adult, i.e., adult-onset GHD, (including adult-onset GHD and idiopathic adult onset-GHD) who was not diagnosed as being GH-deficient as a child. Adult-onset AGHD may be caused by damage or trauma to the pituitary gland. The damage is typically caused by a tumor (e.g., a tumor in and/or around the pituitary gland; or a tumor in the hypothalamus). Pituitary tumors can compress the gland or damage can occur when the tumor is removed via neurosurgery. The pituitary can also be damaged by infection, blood vessel disease, severe head injury, or cranial radiation or chemotherapy for treating tumors of the head and neck. AGHD may be caused by: trauma that occurred in an adult at their birth or soon after their birth; central nervous system infection; tumors of the hypothalamus or pituitary glands; infiltrative or granulomatous disease; cranial irradiation; surgery; or idiopathic causes. GHD in the elderly becomes manifest in decreased quality of life, fatigue, and alteration of body composition. Abnormalities in body composition, bone metabolism, and lipid profile in GH-deficient and hypopituitary adults are distinct from those that occur as the result of normal aging. AGHD includes congenital or acquired GH deficiency in adults, as well as any other adult indication for which GH can be utilized (including where endogenous growth hormone levels in a subject are not necessarily deficient).

Most processes involved in growth of the body are regulated by multiple peptides and hormones, and such peptides and hormones, as well as analogues thereof, have found utility in the treatment of growth hormone-related diseases, disorders and conditions. However, the use of commercially-available growth hormones, has met with less than optimal success in the management of subjects afflicted with such diseases, disorders and conditions. In particular, dose optimization and frequency of dosing is important for peptide and hormone biologics used in the treatment of growth hormone-related diseases and disorders. The fact that growth hormone has a short half-life (e.g., usually less than 4 hours when administered subcutaneously), necessitates frequent (e.g., daily) dosing in order to achieve clinical benefit, which results in difficulties in the management of such patients. Non-compliance with daily growth hormone (GH) injections can lead to loss of treatment effects.

The present invention relates to the enhancement of the safety and tolerability, and the ability to achieve IGF-I levels within a target range in adults with GH deficiency (GHD) after administration of a single dose of the long-acting rhGH analogue, VRS-317, the sequence of which is shown in FIG. 1 (SEQ ID NO: 1). As detailed in the Examples, in a randomized, double-blind, placebo-controlled, single ascending dose study, 50 GHD adults (mean age 45 yr.) were studied in 5 treatment groups of 10 subjects each (8 active, 2 placebo per group). The main outcome measures included adverse events, safety laboratories, VRS-317 pharmacokinetics and pharmacodynamics (including, but not limited to determination of IGF-I and IGFBP-3 concentrations). The results indicate that using a single-dose administration of 0.80 mg/kg of VRS-317, a mean terminal elimination half-life of 131 hours is achieved in subjects. Single VRS-317 doses of 0.05, 0.10, 0.20, 0.40 and 0.80 mg/kg (approximately equivalent to daily rhGH doses of 0.3 to 5.0 μg/kg over 30 days) safely increased the amplitude and duration of IGF-I responses in a dose-dependent manner. After a single 0.80 mg/kg dose, serum IGF-I was maintained in the normal range between −1.5 to 1.5 standard deviations (SD) for a mean of three weeks. No unexpected or serious adverse events were observed in subjects receiving VRS-317. The elimination half-life for VRS-317 is 30-60-fold longer and stimulates more durable IGF-I responses compared to previously studied rhGH products. Prolonged IGF-I responses do not come at the expense of over-exposure to high IGF-I levels. The pharmacokinetic and pharmacodynamics combined with the observed safety profile indicate the potential for safe and effective monthly dosing using VRS-317. The protocols, results, and analysis of this study are discussed further in Examples 1 and 2.

In one aspect, the present invention provides a method of treating growth hormone deficiency (GHD) in a human patient by administering a human growth hormone-XTEN (hGH-XTEN) fusion protein to the patient. In one embodiment, the method comprises administering the hGH-XTEN fusion protein to the patient as a bolus dose. In another embodiment, the bolus dose is a therapeutically effective bodyweight adjusted bolus dose. In one other embodiment, the bolus dose is between about 0.05 mg/kg and about 3.0 mg/kg. In one embodiment, the fusion protein comprises an amino acid sequence having at least about 90% sequence identity to SEQ ID NO:1. In one additional embodiment, the human patient is an adult.

In one aspect, the bolus dose may be administered over a range of doses. It should be noted that where reference is made to the administration of a bolus dose between about a first mg/kg and about a second mg/kg, the “first mg/kg” term may include the first mg/kg value and the “second mg/kg” term may include the second mg/kg value.

In one embodiment, the hGH-XTEN fusion protein comprises (i) an amino acid sequence having at least about 90% sequence identity to SEQ ID NO.1; (ii) the amino acid sequence of SEQ ID NO:1; (iii) an amino acid sequence having at least about 90% sequence identity to SEQ ID NO: 7; (iv) the amino acid sequence of SEQ ID NO: 7; (v) an amino acid sequence having at least about 90% sequence identity to SEQ ID NO:83; or (vi) the amino acid sequence of SEQ ID NO: 83.

In one embodiment, the method of treating GHD in the human patient comprises administering a single dose of an hGH-XTEN fusion protein comprising an amino acid sequence having at least about 90% sequence identity to SEQ ID NO: 1. In another embodiment, the single dose comprises a therapeutically effective bodyweight adjusted bolus dose of the hGH-XTEN fusion protein. In one other embodiment, the bolus dose is between about 0.05 mg/kg and about 3.0 mg/kg, or between about 0.05 mg/kg and about 0.8 mg/kg. In another embodiment, the bolus dose is about 0.05 mg/kg, about 0.1 mg/kg, about 0.2 mg/kg, about 0.4 mg/kg, or about 0.8 mg/kg. In one additional embodiment, the human patient is an adult.

In one other aspect, the bolus dose of the hGH-XTEN fusion protein is administered to a human patient on a regular basis over a suitable time period, which can be finite or indefinite. In one embodiment, the bolus dose is administered every week, every two weeks, every three weeks, or monthly. In other embodiments, the bolus dose is administered once a month, twice a month, three times a month, or four times a month. In another embodiment, the bolus dose is administered about every 7 days, about every 10 days, about every 14 days, about every 21 days, about every 28 days, or about every 30 days. In one embodiment, the bolus dose is administered on a non-daily basis, or is a non-daily bolus dose. In one additional embodiment, the human patient is an adult.

In another aspect, additional bolus doses and ranges of bolus doses of the hGH-XTEN fusion protein for a human patient are suitable. In one embodiment, the bolus dose is between about 0.05 mg/kg and about 0.8 mg/kg, between about 0.8 mg/kg and about 1.2 mg/kg, or between about 0.05 mg/kg and about 3.0 mg/kg. In another embodiment, the bolus dose is selected from the group consisting of about 0.05 mg/kg, about 0.1 mg/kg, about 0.2 mg/kg, about 0.4 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, and about 3.0 mg/kg. In one additional embodiment, the human patient is an adult.

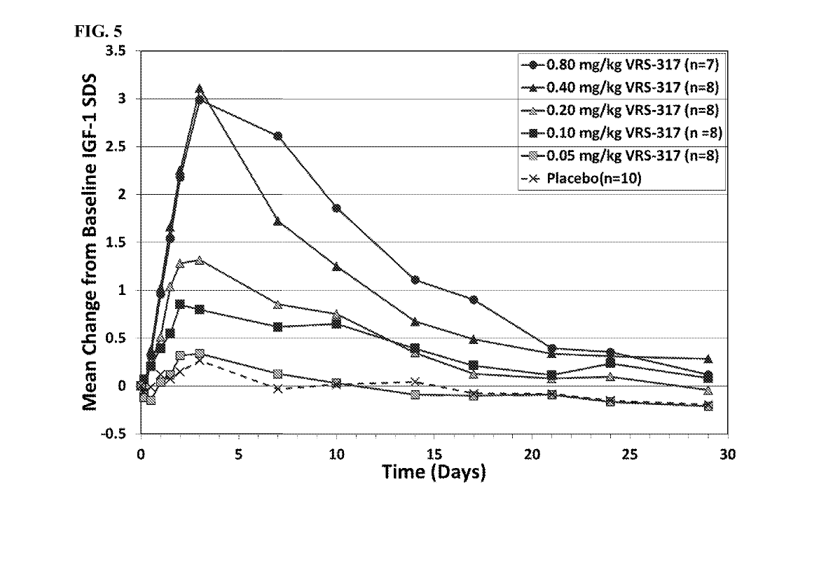

The methods of the present invention are advantageous with respect to IGF-I levels in the human patient following treatment with hGH-XTEN fusion protein. A high level of blood IGF-I is undesirable since high IGF-I is believed to be a risk factor for cancer (Svensson et al. J Clin Endocrin Metab. epub Sep. 26, 2012 as doi:10.1210/jc.2012-2329). IGF-I generation in humans is largely the result of GH signaling and IGF-I is an important mediator for anabolic actions observed during GH therapy (Le Roith et al. (2001). Endocr Rev 22, 53-74). Accordingly, IGF-I is an important pharmacodynamic marker for hGH-XTEN fusion protein bioactivity. In practice, IGF-I responses to GH (e.g., daily rhGH therapy) are interpreted in terms of age- and gender-specific normative data (Vance et al. (1999). N Engl J Med 341, 1206-16; Molitch et al. (2011). J Clin Endocrinol Metab 96, 1587-609). The interpretation is most readily done with the use of IGF-I standard deviation scores (IGF-I SDS). Further, adults with GH deficiency, as with healthy adults, have a range of baseline IGF-I values. Accordingly, IGF-I SDS, corrected for baseline at time 0, can be used to examine potential hGH-XTEN fusion protein dose effects on IGF-I responses. For example, the time course of change in baseline corrected IGF-I SDS by dose group for VRS-317 is shown in FIG. 5.

In one aspect, the present invention provides methods of treatment of GHD in which the human patient maintains an IGF-I response (e.g., as measured by mean IGF-I SDS) in a normal range after administration of the hGH-XTEN fusion protein. For an IGF-I SDS, a normal range is generally between about −1.5 and about 1.5 but can also be between about −2.0 and about 2.0. In one additional embodiment, the human patient is an adult.

It should be noted that where reference is made to an IGF-I SDS between about a first value (e.g., −2.0) and about a second value (e.g., 2.0), the “first value” may include the first value and the “second value” may include the second value.

In one embodiment, the present invention provides a method of treating growth hormone deficiency (GHD) in a human patient by administering an hGH-XTEN fusion protein to the patient, wherein the human patient has a serum IGF-I standard deviation score (SDS) between about −2.0 and about 2.0 following administration. In one embodiment, the method comprises administering the hGH-XTEN fusion protein to the patient as a bolus dose. In another embodiment, the bolus dose is a therapeutically effective bodyweight adjusted bolus dose. In another embodiment, the bolus dose is effective to maintain the patient's serum IGF-I standard deviation score (SDS) between about −2.0 and about 2.0 for (i) at least 7 days; (ii) at least about 10 days; or (iii) at least about 20 days after administration of the bolus dose. In one additional embodiment, the human patient is an adult.

In one embodiment, the invention provides a method of treating human growth hormone deficiency (GHD) in a human subject, comprising administering to the subject with GHD a pharmaceutical composition comprising an effective amount of hGH-XTEN fusion protein having the amino acid sequence set forth in FIG. 1 (SEQ ID NO:1) wherein said amount is at least about 0.05 mg/kg in a single bolus dose, and further wherein said amount is effective to maintain the subject's serum IGF-I SDS between about −1.5 and about 1.5 for (i) at least 7 days; (ii) at least about 10 days; or (iii) at least about 20 days after administration of the single bolus dose of the fusion protein. In one additional embodiment, the human patient is an adult.

In a further embodiment of the method, the effective amount of the hGH-XTEN fusion protein administered to a human patient is at least about 0.1 mg/kg, at least about 0.2 mg/kg, at least about 0.4 mg/kg, at least about 0.8 mg/kg, at least about 1.0 mg/kg, at least about 1.2 mg/kg, at least about 1.4 mg/kg, at least about 1.6 mg/kg, at least about 1.8 mg/kg, at least about 2.0 mg/kg, at least about 2.2 mg/kg, at least about 2.4 mg/kg, at least about 2.6 mg/kg, at least about 2.7 mg/kg, at least about 2.8 mg/kg, or at least 3.0 mg/kg. In one additional embodiment, the human patient is an adult.

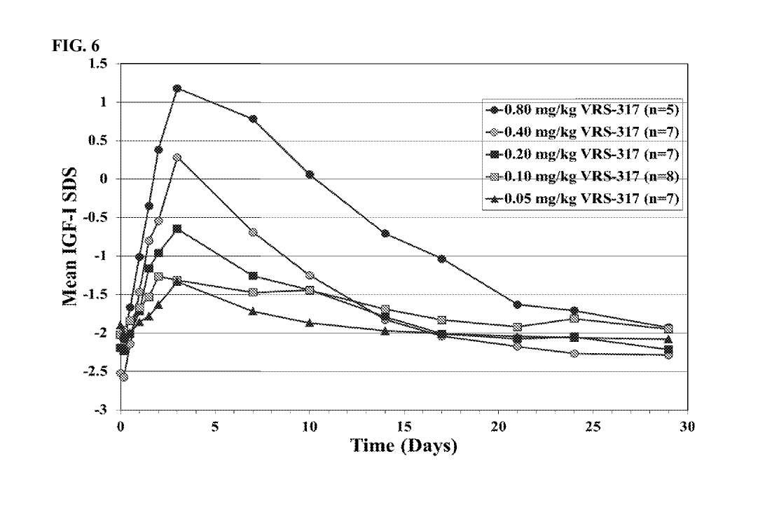

In another embodiment of the method, the effective amount administered to the human patient is between about 0.05 mg/kg and about 3.0 mg/kg. In another embodiment of the method, the effective amount administered is between about 0.2 mg/kg and about 0.8 mg/kg. In another embodiment of the method, the amount of hGH-XTEN fusion protein administered is effective to maintain the subject's serum IGF-I SDS at between about −1.5 and about 1.5 for at least about 15 or at least about 20 days after administration of a single dose of the fusion protein. For example, the mean IGF-I SDS by dose group after administration of VRS-317 is shown in FIG. 6. In one additional embodiment, the human patient is an adult.

The methods of the present invention provides a particular advantage in that that the administration of hGH-XTEN fusion protein provides an observable and prolonged IGF-I response in the human patient (e.g., as measured by IGF-I SDS) that is not accompanied by, or at the expense of, over-exposure to high levels of IGF-I, which is undesirable. In other words, the IGF-I response is maintained at an elevated level that is still considered acceptable by current standards, e.g., as indicated by an IGF-I SDS of 1.5 or less, or an IGF-I SDS of 2.0 or less.

In one embodiment, the invention provides a method for achieving a beneficial effect in a human patient with growth hormone deficiency, comprising the step of administering to the subject a therapeutically-effective amount of a hGH-XTEN fusion protein wherein said administration results in the improvement of one or more biochemical or physiological parameters or clinical endpoints associated with a growth hormone-related disease, disorder or condition. The effective amount produces a beneficial effect in helping to treat (e.g., cure or reduce the severity) the deleterious effects of a growth hormone-related disease, disorder or condition. In some cases, the method for achieving a beneficial effect includes administering a therapeutically effective amount of a hGH-XTEN fusion protein composition to treat a subject with a growth hormone-related disease, disorder, or condition, including, but not limited to, congenital or acquired GH deficiency in adults (including adults who experienced a growth hormone-related disorder as children, such as Turner's Syndrome, Prader-Willi Syndrome, idiopathic short stature, or intrauterine growth retardation); and adults experiencing chronic renal failure, AIDS wasting, obesity, multiple sclerosis, aging, fibromyalgia, Crohn's disease, ulcerative colitis, muscular dystrophy, low muscle mass (e.g. bodybuilding), low bone density, or any other indication for which GH can be utilized (but for which endogenous growth hormone levels in a subject are not necessarily deficient). In one additional embodiment, the human patient is an adult.

The methods of the invention include the administration to a human patient of successive or consecutive doses of a therapeutically effective amount of the hGH-XTEN for a period of time sufficient to achieve and/or maintain the desired parameter or clinical effect, and such consecutive doses of a therapeutically effective amount establishes the therapeutically effective dose regimen for the hGH-XTEN; i.e., the schedule for consecutively administered doses of the fusion protein composition, wherein the doses are given in therapeutically effective amounts to result in a sustained beneficial effect on any clinical sign or symptom, aspect, measured parameter or characteristic of a metabolic disease state or condition, including, but not limited to, those described herein. In one embodiment of the method, the parameters include but are not limited to IGF-I concentration, ratio of IGF-I/IGFBP-3, IGFBP3 concentration, change in weight, lean body mass, change in body mass index, total body fat (adipose fat/tissue), trunk fat, response to insulin challenge, rate of division of chondrocytes, chondrocyte numbers, bone density, bone age, bone growth, bone turnover, increase in epiphyseal plate width, reduction in cholesterol, reduction in triglycerides, and reduction in LDL. In one additional embodiment, the human patient is an adult.

In one embodiment, the pharmaceutical composition is administered at a therapeutically effective dose. In another embodiment, the pharmaceutical composition is administered using multiple consecutive doses using a therapeutically effective dose regimen (as defined herein) for the length of the dosing period.

A therapeutically effective amount of the hGH-XTEN varies according to factors such as the disease state, age, sex, and weight of the individual, and the ability of the antibody or antibody portion to elicit a desired response in the individual. A therapeutically effective amount is also one in which any toxic or detrimental effects of the hGH-XTEN are outweighed by the therapeutically beneficial effects.

In one embodiment, the method comprises administering to a human patient with GHD at least two therapeutically effective bodyweight adjusted bolus doses of a human growth hormone hGH-XTEN fusion protein having at least about 90%, or at least about 95%, or at least about 96%, or at least about 97%, or at least about 98%, or at least about 99% sequence identity to the sequence as set forth in FIG. 1 (SEQ ID NO:1), wherein said administration of said bolus doses is separated by at least about 7 days, at least about 10 days, at least about 14 days, at least about 21 days, at least about 28 days, or at least about monthly and wherein the therapeutically effective bodyweight adjusted bolus dose of hGH-XTEN fusion protein is selected from the group consisting of: about 0.05 mg/kg, about 0.1 mg/kg, about 0.2 mg/kg, about 0.4 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, and 3.0 mg/kg. In one additional embodiment, the human patient is an adult.

In another embodiment, the therapeutically effective bodyweight adjusted bolus doses of hGH fusion protein are administered subcutaneously to the human patient. In some embodiments, the human patient has a serum IGF-I standard deviation (SD) score of greater than about −2.0, greater than about −1.5, greater than about −1.0, greater than about −0.5, or greater than about 0, greater than about 0.5, greater than about 1.0, greater than about 1.5, greater than about 1.6, greater than about 1.7, greater than about 1.8, or greater than about 1.9 following administration of the hGH-XTEN. In one embodiment, the hGH-XTEN fusion protein has the amino acid sequence shown as set forth in FIG. 1 (SEQ ID NO:1). In one additional embodiment, the human patient is an adult.

In one embodiment, the invention provides a method of treating human growth hormone deficiency (GHD) in a human subject, comprising administering to the subject with GHD a pharmaceutical composition comprising an effective amount of hGH-XTEN fusion protein having the amino acid sequence set forth in FIG. 1 (SEQ ID NO:1) wherein said amount is at least about 0.05 mg/kg in a single bolus dose, and further wherein said amount is effective to maintain the subject's serum IGF-I SD score between about −1.5 and about 1.5 for at least 10 days after administration of the single bolus dose of the fusion protein. In a further embodiment of the method, the amount administered is at least 0.2 mg/kg. In another embodiment of the method, the amount administered is between about 0.05 mg/kg and about 3.0 mg/kg. In another embodiment of the method, the amount administered is between about 0.2 mg/kg and about 0.8 mg/kg. In another embodiment of the method, the amount administered is effective to maintain the subject's serum IGF-I SD score between about −1.5 and about 1.5 for at least 20 days after administration of a single dose of the fusion protein. In one additional embodiment, the human patient is an adult.

In another embodiment, the invention provides a method of treating human growth hormone deficiency (GHD) in a human subject, comprising administering to the subject with GHD a pharmaceutical composition comprising an effective amount of hGH-XTEN fusion protein having the amino acid sequence set forth in FIG. 1 (SEQ ID NO:1) wherein said amount is at least 0.05 mg/kg in a single bolus dose and is effective to maintain a plasma concentration of said fusion protein at more than about 10 ng/mL for a period of at least 10 days after administration of the single bolus dose of the fusion protein. In a further embodiment of the method, the amount administered is at least 0.2 mg/kg. In another embodiment of the method, the amount administered is between about 0.05 mg/kg and about 3.0 mg/kg. In another embodiment of the method, the amount administered is between about 0.2 mg/kg and about 0.8 mg/kg. In another embodiment of the method, the amount administered is effective to maintain a plasma concentration of said fusion protein at more than about 10 ng/mL for a period of at least 20 days after administration of the single bolus dose of the fusion protein. In another embodiment of the method, the amount administered is effective to maintain a plasma concentration of said fusion protein at more than about 10 ng/mL for a period of at least 30 days after administration of the single bolus dose of the fusion protein. In another embodiment of the method, the amount administered is effective to maintain a plasma concentration of said fusion protein at more than about 100 ng/mL for a period of at least 10 days after administration of the single bolus dose of the fusion protein. In one additional embodiment, the human patient is an adult.

In one embodiment, the invention provides a method of treating human growth hormone deficiency (GHD) in a human subject comprising administering to the subject with GHD a pharmaceutical composition comprising an effective amount of hGH-XTEN fusion protein having the amino acid sequence set forth in FIG. 1 (SEQ ID NO:1) wherein said amount is at least 0.05 mg/kg in a single bolus dose and is effective in increasing the subject's plasma IGF-I SD score by at least 0.5 above the subject's baseline IGF-I SD score without causing a clinically significant level of side-effects selected from the group consisting of headache, arthralgia, myalgia, edema, nausea, and muscle fatigue after administration of the single bolus dose of the fusion protein. As used herein, “clinically significant level of side-effects” means that the side-effects are not unexpected or are not serious adverse events. Side-effects that are mild and transient, even if one of headache, arthralgia, myalgia, edema, nausea, and muscle fatigue or those otherwise known to be associated with the administration of growth hormone, would not be considered a clinically significant level. In another embodiment of the method of treating GHD, the amount administered is at least about 0.2 mg/kg. In another embodiment of the method of treating GHD, the amount administered is between about 0.05 mg/kg and about 3.0 mg/kg. In another embodiment of the method of treating GHD, the amount administered is between about 0.2 mg/kg and about 0.8 mg/kg. In another embodiment of the method of treating GHD, the amount administered is between about 0.2 mg/kg and about 3.0 mg/kg. In another embodiment of the method of treating GHD, the single bolus dose is administered subcutaneously. In another embodiment of the method of treating GHD, the pharmaceutical composition comprising the hGH-XTEN fusion protein is administered using two or more consecutive doses. In one additional embodiment, the human patient is an adult.

In one other aspect, the methods of the present invention related to improved therapeutic regimens for GHD therapy comprise improving lipid metabolism parameters in a subject in need, e.g., a human patient with GHD. In one embodiment, the method of improving lipid parameters in a subject in need comprises administering an at least two therapeutically effective bodyweight adjusted bolus doses of a hGH-XTEN fusion protein, wherein the administration of said bolus doses is separated by at least about 7 days, or at least about 10 days, at least about 14 days, at least about 21 days, at least about 28 days, or at least about monthly and wherein the bolus doses provide an improvement in lipid parameters in said subject. In one embodiment, the improvement in lipid parameters is an improvement selected from the group consisting of lower triglyceride levels, lower cholesterol, and lower LDL levels. In one additional embodiment, the human patient is an adult.

The invention provides methods to establish a dose regimen for the hGH-XTEN pharmaceutical compositions of the invention for human patients. The methods include administration of consecutive doses of a therapeutically effective amount of the hGH-XTEN composition using variable periods of time between doses to determine that interval of dosing sufficient to achieve and/or maintain the desired parameter, blood level or clinical effect; such consecutive doses of a therapeutically effective amount at the effective interval establishes the therapeutically effective dose regimen for the hGH-XTEN for a GHD condition. Thus, in one aspect, the invention provides an hGH-XTEN composition for use in a treatment regimen that is therapeutically effective for human growth hormone deficiency (GHD). In one additional embodiment, the human patient is an adult.

In another aspect, the invention provides an hGH-XTEN fusion protein for use in a treatment regimen for human growth hormone deficiency (GHD), which regimen comprises administering a hGH-XTEN fusion protein to a human patient.

In one embodiment, the treatment regimen comprises administering a bolus dose of the hGH-XTEN fusion protein to the human patient. In another embodiment, the bolus dose is (i) a therapeutically effective bodyweight adjusted bolus dose; and/or (i) between about 0.05 mg/kg and about 3.0 mg/kg. In one other embodiment, the treatment regimen comprises administering the bolus dose every week, every two weeks, every three weeks, or monthly. In one additional embodiment, the treatment regimen comprises subcutaneous administration of the bolus dose. In one additional embodiment, the human patient is an adult.

In one embodiment, the regimen comprises administering at least two bolus doses of the hGH-XTEN fusion protein to a human patient wherein the dosage is about 0.05 mg/kg, about 0.1 mg/kg, about 0.2 mg/kg, about 0.4 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, and 3.0 mg/kg. In one additional embodiment, the human patient is an adult. In another embodiment, the dosage is administered as at least two bolus doses wherein the administration of said bolus doses is separated by at least about 7 days, or at least about 10 days, at least about 14 days, at least about 21 days, at least about 28 days, or at least about monthly.

In one embodiment of the treatment regimen, the administration of said bolus doses is separated by at least about one month, at least about 31 days, at least about 30 days, at least about 29 days, at least about 28 days, at least about 27 days, at least about 26 days, at least about 25 days, at least about 24 days, at least about 23 days, at least about 22 days, at least about 21 days, at least about 20 days, at least about 19 days, at least about 18 days, at least about 17 days, at least about 16 days, at least about 15 days, at least about 14 days, at least about 13 days, at least about 12 days, at least about 11 days, at least about 10 days, at least about 9 days, at least about 8 days, at least about 7 days, at least about 6 days, at least about 5 days, at least about 4 days, at least about 3 days, or at least about 2 days. In another embodiment, the present invention provides a consecutive dose regimen wherein each bolus dose is administered every week (or weekly), every two weeks, every three weeks, every four weeks, or monthly.

In one embodiment of the hGH-XTEN composition for use in a treatment regimen, the hGH-XTEN fusion protein comprises the amino acid sequence shown as set forth in FIG. 1 (SEQ ID NO:1). In one embodiment, the therapeutically effective dose treatment regimen comprises the administration of at least two therapeutically effective bodyweight adjusted bolus doses to a subject, wherein the doses are administered subcutaneously.

In general, a “bolus dose” is a dose administered within a short period of time. In another embodiment, the bolus dose is administered within about 1 to about 30 minutes, about 1 to about 20 minutes, about 1 to about 15 minutes, about 1 to about 10 minutes, or about 1 to about 5 minutes. In one embodiment, the bolus dose is administered within about 1 to about 5 minutes. In one other embodiment, the bolus does is a subcutaneous bolus dose.

In another aspect, the treatment regimen results in the human patient exhibiting an improvement in the serum IGF-I standard deviation score (SDS) following administration of a bolus dose. In one embodiment, the IGF-I SDS is between about −2.0 and about 2.0 in the patient following administration of the bolus dose. In another embodiment, the IGF-I SDS is selected from the group consisting of greater than about −2.0, greater than about −1.5, greater than about −1.0, greater than about −0.5, greater than about 0, greater than about 0.5, greater than about 1.0, and greater than about 1.5. In one additional embodiment, the human patient exhibits said IGF-I SDS following administration of the bolus dose, wherein the administration is selected from the group consisting of weekly, every two weeks, every three weeks, and monthly.

In another aspect, the treatment regimen results in normalization of IGF-I concentration in the human patient following administration. In one embodiment, the regimen results in an IGF-I concentration that is normalized for at least about 7 days, or at least about 10 days, or at least about 14 days, at least about 16 days, at least about 17 days, or at least about 21 days following the administration of the first or second dose.

In one embodiment, the regimen results in a serum IGF-I concentration that is normalized for at least about 7 days, or at least about 10 days, or at least about 14 days, at least about 17 days, or at least about 21 days following the administration of the first or second dose. As would be appreciated by one of ordinary skill in the art, “normalized” would vary according to factors such as the disease state, age, sex, and weight of the individual. In another embodiment, the regimen results in a serum IGF-I standard deviation (SD) score of greater than about −2.0, greater than about −1.5, greater than about −1.0, greater than about −0.5, or greater than about 0 following administration of the first or second dose.

In one other aspect, the treatment regimen results in a clinically significant reduction in the patient in at least one parameter related to the GHD evaluation after administration of a bolus dose. In one embodiment, the treatment regimen results in a reduction in the patient of at least one parameter selected from serum cholesterol, serum triglycerides, and serum low density lipoprotein (LDL) after administration of the bolus dose. In another embodiment, the treatment regimen comprises administration of a bolus dose weekly, every two weeks, every three weeks, or monthly.

In another embodiment, the regimen results in a clinically significant reduction in the patient in at least one parameter selected from serum cholesterol, serum triglycerides, and serum LDL after administration of the first or second bolus dose. In another embodiment, the regimen results in an AUC of at least about 11,861 ng-hr/mL, or at least about 33,375 ng-hr/mL, or at least about 91,006 ng-hr/mL, or at least about 241,288 ng-hr/mL, or at least about 402,543 ng-hr/mL after administration of the first or second bolus dose. In another embodiment of the regimen, the human patient achieves an improvement after two or more bolus doses in at least one parameter selected from bone density, bone growth, and increase in epiphyseal plate width. In one other embodiment, the foregoing improvement(s) is at least about 10%, or at least about 20%, or at least about 30%, or at least about 40%, or at least about 50%, or at least about 60%, or at least about 70%, or at least about 80%, or at least about 90%. In another embodiment, the foregoing % improvement(s) is similar to, or not inferior to, an improvement achieved by an hGH not linked to XTEN and administered daily using daily dosage equivalent amounts of hGH.

In another aspect, the present invention provides methods of treating human growth hormone deficiency (GHD) with a therapeutically effective amount of an hGH-XTEN fusion protein at a dosage that is equivalent to, or equivalent to less than, an effective amount of a corresponding hGH (not linked to XTEN) administered daily. In one embodiment, the present invention provides methods of treating human growth hormone deficiency (GHD), comprising administering to a human patient a therapeutically effective amount of a human growth hormone

(hGH)—XTEN fusion protein, wherein the dosage of the hGH fusion protein is equivalent to an amount that is less than about 2 μg hGH/kg/day to about 12 μg hGH/kg/day. In one embodiment, the human patient is an adult.

In one additional aspect, the present invention provides methods of treating human growth hormone deficiency (GHD), comprising administering to a human patient with GHD an hGH-XTEN fusion protein at a dosage that is below or less than an equivalent daily dose of recombinant hGH (e.g., a recommended daily dose of rhGH).

In one embodiment, the method comprises administering an hGH-XTEN fusion protein as a bolus dose that is equivalent to less than an hGH/kg/day dosage that is (i) between about 2 μg hGH/kg/day and about 20 μg hGH/kg/day; or (ii) between about 2 μg hGH/kg/day and about 12 μg hGH/kg/day.

In one aspect, the bolus dose may be administered over a range of doses that are equivalent to less than an hGH/kg/day dosage. It should be noted that where reference is made to a bolus dose that is equivalent to less than an hGH/kg/day dosage that is between about a first μg hGH/kg/day and about a second μg hGH/kg/day, the “first μg hGH/kg/day” term may include the first μg hGH/kg/day value and the “second μg hGH/kg/day” term may include the second μg hGH/kg/day value.

In another embodiment, the bolus dose is a therapeutically effective bodyweight adjusted bolus dose of the hGH-XTEN fusion protein. In one other embodiment, the bolus dose is equivalent to less than an hGH/kg/day dosage administered over about 7 days, about 14 days, about 21 days, about 28 days, or about 30 days. In one embodiment, the present invention provides methods of treating human growth hormone deficiency (GHD), comprising administering to a human patient with GHD at least two therapeutically effective bodyweight adjusted bolus doses of a human growth hormone hGH-XTEN fusion protein, wherein the administration of said bolus doses is separated by at least about one week, and wherein the dosage of the hGH-XTEN fusion protein is equivalent to (i) less than about 0.3 μg hGH/kg/day to about 18.0 μg hGH/kg/day; or (ii) less than about 0.3 μg hGH/kg/day to about 18.6 μg hGH/kg/day. In another embodiment, the dosage of the hGH-XTEN fusion protein is equivalent to (i) less than about 2 μg hGH/kg/day to about 12 μg hGH/kg/day; or (ii) less than about 2 μg hGH/kg/day to about 20 μg hGH/kg/day. In another embodiment, the administration of said bolus doses is separated by at least about 7 days, at least about 10 days, at least about 14 days, at least about 21 days, at least about 28 days, or at least about monthly. In one embodiment, the dosage equivalent amount of hGH is less than about 4.8 μg/kg/day. In one additional embodiment, the human patient is an adult.

In another embodiment, the bolus dose is equivalent to an hGH/kg/day dosage that is less than about 2 μg hGH/kg/day. In another embodiment, the dosage is equivalent to less than about 0.3 μg hGH/kg/day, about 0.6 μg hGH/kg/day, about 1.2 μg hGH/kg/day, about 2.0 μg hGH/kg/day, about 2.4 μg hGH/kg/day, about 4.0 μg hGH/kg/day, about 4.8 μg hGH/kg/day, about 6.0 μg hGH/kg/day, about 6.2 μg hGH/kg/day, about 7.4 μg hGH/kg/day, about 8.0 μg hGH/kg/day, about 8.6 μg hGH/kg/day, about 9.8 μg hGH/kg/day, about 10 μg hGH/kg/day, about 11.1 μg hGH/kg/day, about 12 μg hGH/kg/day, about 12.4 μg hGH/kg/day, about 13.6 μg hGH/kg/day, about 14 μg hGH/kg/day, about 14.8 μg hGH/kg/day, about 16.0 μg hGH/kg/day, about 16.8 μg hGH/kg/day, about 17.4 μg hGH/kg/day, about 18 μg hGH/kg/day, about 18.6 μg hGH/kg/day, or about 20 μg hGH/kg/day. In one additional embodiment, the human patient is an adult.

In one other embodiment, the bolus dose is equivalent to less than an hGH/kg/day dosage administered over about 7 days, about 14 days, about 21 days, about 28 days, or about 30 days.

In one other embodiment, method comprises administering to the patient a therapeutically effective bodyweight adjusted bolus dose of a human growth hormone-XTEN (hGH-XTEN) fusion protein comprising an amino acid sequence having at least about 90% sequence identity to the sequence set forth in FIG. 1 (SEQ ID NO.1), wherein the mass of human growth hormone administered to the patient is equivalent to less than 0.006 mg/kg/day. In another embodiment, the mass of human growth hormone administered to the patient is equivalent to between about 0.0003 mg/kg/day and about 0.005 mg/kg/day. In one other embodiment, the method comprises monthly dosing of the patient with the hGH-XTEN. In one additional embodiment, the human patient is an adult.

In yet another embodiment, the hGH-XTEN fusion protein comprises an amino acid sequence shown as set forth in FIG. 1 (SEQ ID NO:1). In other embodiments, the administration is subcutaneous administration.

In another aspect, the present invention provides methods of normalizing serum IGF-I levels in a subject in need thereof. In one embodiment, the method comprises administering the hGH-XTEN fusion protein to a human patient as a bolus dose that is effective in increasing the patient's IGF-I SDS by at least 0.5 or at least 1.0 above the subject's baseline IGF-I SDS. In another embodiment, the increase in IGF-I SDS is achieved in the absence of a clinically significant level of side-effects selected from the group consisting of headache, arthralgia, myalgia, edema, nausea, and muscle fatigue after administration of the bolus dose. In one additional embodiment, the bolus dose is (i) a therapeutically effective bodyweight adjusted bolus dose; and/or (ii) is administered subcutaneously.

In one other embodiment, the method comprises administering to the subject with GHD at least two therapeutically effective bodyweight adjusted bolus doses of a human growth hormone hGH-XTEN fusion protein, wherein the bolus dose provides a normal serum IGF-I level in said subject. In another embodiment, the administration of said bolus doses is separated by at least about 7 days, or at least about 10 days, at least about 14 days, at least about 21 days, at least about 28 days, or at least about monthly. In one other embodiment of the method, the administration of said bolus doses results in a normalization of serum IGF-I levels in the subject for at least about 5 days, or at least about 10 days, or at least about 14 days, or at least about 17 days, or at least about 21 days. FIG. 6 provides an illustration of normalization of IGF-I in various patients. In one other embodiment, a normal serum IGF-I level is characterized by a serum IGF-I standard deviation (SD) that is above about −2.0; above about −1.5; above about −1.0; above about 0; above about 0.5; above about 1.0; or above about 1.5. In another embodiment, a normal serum IGF-I level is characterized by a serum IGF-I standard deviation (SD) that is between about −1.5 and about 1.5; between about −1.5 and about 1.0; between about −1.5 and about 0.5; between about −1.5 and about 0; between about −1.5 and about −0.5; and between about −1.5 and about −1.0. In one additional embodiment, the human patient is an adult.

In another embodiment, the subject is a human subject having GHD. In an additional embodiment, the administration is subcutaneous administration. In one other embodiment, the therapeutically effective bodyweight adjusted bolus dose of hGH-XTEN fusion protein is selected from the group consisting of: about 0.05 mg/kg, about 0.1 mg/kg, about 0.2 mg/kg, about 0.4 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, and 3.0 mg/kg. In one additional embodiment, the human patient is an adult. In an additional embodiment, the extent of normalization of IGF-I serum levels is dependent on the dose of the therapeutically effective bodyweight adjusted bolus dose of hGH fusion protein. In one other embodiment, the duration of the IGF-I normalization increases with the therapeutically effective bodyweight adjusted bolus dose of hGH fusion protein.

In another embodiment, the present invention provides an hGH-XTEN fusion protein for use as a medicament, or for the treatment of GHD. In another embodiment, the present invention provides the use of an hGH-XTEN fusion protein for the manufacture of a medicament for treating GHD in a human patient with GHD. In one other embodiment, the present invention provides the use of the fusion protein having the sequence set forth in FIG. 1

(SEQ ID NO:1) in the manufacture of a medicament for the treatment of GHD. In other embodiments, the hGH-XTEN fusion protein is provided in a therapeutically effective bodyweight adjusted dose suitable for bolus administration. In some embodiments, the therapeutically effective bodyweight adjusted bolus dose of hGH-XTEN fusion protein is selected from the group consisting of: about 0.05 mg/kg, about 0.1 mg/kg, about 0.2 mg/kg, about 0.4 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, and 3.0 mg/kg. In one additional embodiment, the human patient is an adult. In another embodiment, the therapeutically effective bodyweight adjusted bolus of hGH-XTEN fusion protein is administered subcutaneously. In some embodiments, the human patient has a serum IGF-I standard deviation (SD) score of greater than about −2.0, greater than about −1.5, greater than about −1.0, greater than about −0.5, greater than about 0, greater than about 0.5, greater than about 1.0, greater than about 1.5, greater than about 1.6, greater than about 1.7, greater than about 1.8, or greater than about 1.9 following administration of the hGH-XTEN fusion protein. In one embodiment, the hGH-XTEN fusion protein comprises an amino acid sequence shown as set forth in FIG. 1 (SEQ ID NO:1).

In another aspect, the present invention provides hGH-XTEN fusion protein-based therapeutic agents for treating diseases or conditions related to growth hormone deficiency (GHD). For the prevention, treatment or reduction in the severity of a given disease or condition, the appropriate dosage of a therapeutic agent of the invention will depend on the type of disease or condition to be treated, as defined above, the severity and course of the disease or condition, whether the agent is administered for therapeutic purposes, previous therapy, the patient's clinical history and response to the agent, and the discretion of the attending physician.

In another aspect, the present invention provides a method for the delaying or slowing down of the progression of a disease or condition related to GHD. In one embodiment, the method comprises administering to subject diagnosed with the disease, condition, or disorder, an effective amount of an hGH-XTEN fusion protein. In another aspect, the invention provides a method for treating or ameliorating indicia of a disease or condition related to GHD. In one embodiment, the method comprises administering an effective amount of an hGH-XTEN fusion protein to a subject at risk of the disease or condition, wherein the hGH-XTEN fusion protein is effective against the development of indicia of the disease or condition.

In one additional aspect, the hGH-XTEN fusion proteins provide an ameliorative effect against the development of, or the progression of, clinical and/or histological and/or biochemical and/or pathological indicia (including both symptoms and signs) of diseases or conditions related to GHD in a human subject. In one embodiment, the disease or condition is GHD. In one embodiment, the indicia include an increased level of body fat (especially central or trunk adiposity, i.e, the waist), anxiety and depression, lethargy, changes in mood, feelings of isolation from others, a lack of motivation, elevated levels of cholesterol in the blood (e.g., abnormally high levels of low-density lipoproteins when compared to high density lipoproteins), elevated levels of triglycerides in the blood, decreased sexual function and interest, fatigue, decreased lean muscle mass, decreased extracellular fluid volume, decreased muscle strength, decreased physical energy and stamina, and reduced bone density. In another embodiment, the subject is at risk for a disease of condition related to GHD. In general, a subject at risk will previously have incurred some damage to the pituitary gland and/or the hypothalamus. In one embodiment, the subject at risk was previously diagnosed as having a tumor associated with the pituitary gland, and/or underwent surgery, chemotherapy, or radiation therapy to treat the tumor. In another embodiment, the subject at risk previously had or presently has a reduced blood supply to the pituitary gland. In one other embodiment, the subject at risk previously suffered cranial ablation or has a history of head trauma. In some embodiments, the subject at risk previously or presently suffers from a hypothalamic-pituitary disease or disorder.

The efficacy of the treatment of diseases and conditions described herein (including GHD) can be measured by various assessments commonly used in evaluating GHD. For example, the health of hormone-secreting glands can be evaluated by, but not limited to, e.g., IGF-I standard deviation score (SDS), growth hormone stimulation test (GHST), growth hormone releasing hormone (GHRH), stimulation tests, monitoring or measurement of endogenous hGH pulses, IGF-I levels, IGF-I binding protein levels, other blood or biochemical tests (e.g., total cholesterol, low-density lipoprotein (LDL) cholesterol, high-density lipoprotein (HDL) cholesterol, triglyceride, and lipids).

In one additional aspect, the present invention provides methods of increasing the efficacy of human growth hormone (hGH) therapy in a human patient. In another aspect, the present invention provides methods of determining a subsequent dose of an hGH-XTEN fusion protein administered over a subsequent dosage period when treating a human patient with GHD with the hGH-XTEN fusion protein. The “dosage period” means the time between the administration of a bolus dose (e.g., initial dose) and the next successive administration of a bolus dose (e.g., subsequent dose). The dosage period may change with one or more further successive dose or doses, or may remain constant.

In one embodiment, the foregoing methods of increasing efficacy comprise the step of monitoring the IGF-I standard deviation score (SDS) in a plasma or serum sample obtained from the patient during an initial dosage period of administration of an initial dose of human growth hormone-XTEN (hGH-XTEN) fusion protein. In one embodiment, the hGH-XTEN fusion protein comprising an amino acid sequence having at least about 90% sequence identity to SEQ ID NO:1. In another embodiment, the method further comprises the step of determining a subsequent dose of hGH-XTEN fusion protein administered over a subsequent dosage period based on the IGF-I SDS observed during the initial dosage period. In one additional embodiment, the method further comprises administering the subsequent dose over a subsequent dosage period. In one other embodiment, the subsequent dose improves the efficacy of the treatment during the subsequent dosage period. In another embodiment, the subsequent dose is higher, lower, or equivalent to the initial dose. The initial dose or subsequent dose may be any of the bolus doses described herein. In one additional embodiment, the subsequent dosage period is longer, shorter, or equivalent to the initial dosage period. The initial dosage period or subsequent dosage period may be any of the periods of time described herein (e.g., weekly, every two weeks, etc. or every 7 days, every 10 days, every 14 days, etc.).

### VII). Dosage Forms and Pharmaceutical Compositions

In another aspect, the present invention provides bolus doses or dosage forms comprising an hGH-XTEN fusion protein described herein.

In one embodiment, the bolus dose or dosage of an hGH-XTEN fusion protein comprises a therapeutically effective bodyweight adjusted bolus dose for a human patient. In one other embodiment, the bolus dose or dosage comprises between about 0.05 mg/kg and about 3.0 mg/kg of hGH-XTEN fusion protein. In one additional embodiment, the human patient is an adult.

In one other embodiment, the bolus dose or dosage of hGH-XTEN fusion protein is selected from the group consisting of about 0.05 mg/kg, about 0.1 mg/kg, about 0.2 mg/kg, about 0.4 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, and 3.0 mg/kg. In one additional embodiment, the human patient is an adult.

In other embodiments, the bolus dose or dosage is (i) for use in treating human GHD in a subject in need, e.g., a human patient; and/or (ii) formulated for subcutaneous administration. In one other embodiment, the hGH-XTEN fusion protein comprises the amino acid sequence shown as set forth in FIG. 1 (SEQ ID NO:1). In one embodiment, the bolus dose or dosage form is a pharmaceutical composition comprising the fusion protein having the sequence as set forth in FIG. 1 (SEQ ID NO:1) and a pharmaceutically acceptable carrier.

In another embodiment, the invention provides kits, comprising packaging material and at least a first container comprising the pharmaceutical composition of the foregoing embodiment and a label identifying the pharmaceutical composition and storage and handling conditions, and a sheet of instructions for the preparation and/or administration of the pharmaceutical compositions to a subject.

In one additional aspect, the present invention provides compositions, pharmaceutical compositions, and dose amounts of an hGH-XTEN fusion protein. In one other embodiment, the pharmaceutical composition or dose amount comprises a fusion protein having the sequence as set forth in FIG. 1 (SEQ ID NO:1), or a sequence having at least about 90% sequence identity to the sequence of SEQ ID NO.1. In another embodiment, the dose amount is for a human patient based upon the weight of the patient. In one other embodiment, the human patient is an adult. The weight of the adult human patient can range from about 45 kg to about 120 kg. In one additional embodiment, the hGH-XTEN fusion protein is provided in the pharmaceutical composition, composition, or dose amount as a certain quantity. In another embodiment, the hGH-XTEN fusion protein is provided in an amount (i) between about 2.25 mg to about 6 mg; (ii) between about 4.5 mg and about 12 mg; (iii) between about 9 mg and about 24 mg; (iv) between about 18 mg and about 48 mg; (v) between about 36 mg and about 96 mg; (vi) between about 45 mg and about 120 mg; (vii) between about 54 mg and about 144 mg; (viii) between about 63 mg and about 168 mg; (ix) between about 72 mg and about 192 mg; (x) between about 81 mg and about 216 mg; (xi) between about 90 mg and about 240 mg; (xii) between about 99 mg and about 264 mg; (xiii) between about 108 mg and about 288 mg; (xiv) between about 117 mg and about 312 mg; (xv) between about 121.5 mg and about 324 mg; (xvi) between about 126 mg and about 336 mg; or (xvii) between about 135 mg and about 360 mg. In one other embodiment, the pharmaceutical composition or dose amount further comprises a pharmaceutically acceptable carrier.

It should be noted that where reference is made to a composition, pharmaceutical composition or dose amount comprising an amount of hGH-XTEN fusion protein between about a first mg and about a second mg, the “first mg” term may include the first mg value and the “second mg” term may include the second mg value.

### VIII). Articles of Manufacture

In one aspect, the present invention also provides kits and articles of manufacture containing materials useful for the treatment, prevention and/or diagnosis of disease (e.g., GHD). In another embodiment, the invention provides kits, comprising packaging material and at least a first container comprising a dosage form or pharmaceutical composition of the foregoing embodiment and a label identifying the dosage form or pharmaceutical composition and storage and handling conditions, and a sheet of instructions for the reconstitution and/or administration of the dosage form or pharmaceutical compositions to a subject. In one other embodiment, the kit includes a container and a label, which can be located on the container or associated with the container. The container may be a bottle, vial, syringe, cartridge (including autoinjector cartridges), or any other suitable container, and may be formed from various materials, such as glass or plastic. The container holds a composition having an hGH-XTEN fusion protein as described herein, and may have a sterile access port. Examples of containers include a vial with a stopper that can be pierced by a hypodermic injection needle. The kits may have additional containers that hold various reagents, e.g., diluents, preservatives, and buffers. The label may provide a description of the composition as well as instructions for the intended use.

In one other aspect, the container is a pre-filled syringe. In one embodiment, the syringe is pre-filled with a composition having an hGH-XTEN fusion protein as described herein. In one additional aspect, the present invention provides containers of the composition having a hGH-XTEN fusion protein as described herein, wherein the container is suitable for autoinjection of the composition. In one embodiment, the container is a cartridge. In another embodiment, the container is a cartridge in an autoinjection pen. Those of ordinary skill in the art will appreciate that other suitable autoinjection devices may be used for the present invention. In some embodiments, the autoinjection device comprises a spring-loaded syringe within a cylindrical housing that shields the needle tip prior to injection. In one embodiment, the patient depresses a button on the device and the syringe needle is automatically inserted to deliver the contents.

In another embodiment, the device is a gas jet autoinjection device. In other embodiments, the gas jet device comprises a cylinder of pressurized gas but the needle is absent. Upon activation, the device propels a fine jet of liquid through the skin without the use of a needle. In one other embodiment, the device is an iontophoresis device or electromotive drug administration (EMDA) device (e.g., use of a small electric charge to deliver an agent through the skin without the use of a needle).

The kit has at least one container that includes a molecule comprising an hGH-XTEN fusion protein described herein as the active agent. The container may comprise an hGH-XTEN fusion protein dosage form or pharmaceutical composition. A label may be provided indicating that the dosage form or composition may be used to treat a disease. The label may also provide instructions for administration to a subject in need of treatment. The kit may further contain an additional container having a pharmaceutically-acceptable buffer, such as bacteriostatic water for injection (BWFI), phosphate-buffered saline, Ringer's solution and dextrose solution. Finally, the kit may also contain any other suitable materials, including other buffers, diluents, filters, needles, and syringes.

In one aspect, the present invention provides a kit comprising a container which holds a pharmaceutical composition for administration to a human patient comprising a human growth hormone-XTEN (hGH-XTEN) fusion protein. In one embodiment, the hGH-XTEN fusion protein comprises an amino acid sequence having at least about 90% sequence identity to the sequence set forth in FIG. 1 (SEQ ID NO.1). In another embodiment, the kit further comprises a package insert associated with said container. In one other embodiment, the package insert indicates that said composition is for the treatment of growth hormone deficiency by administration of more than one dose of the composition. In one embodiment, the administration is an administration of an initial dose of between about 0.05 mg/kg and about 3.0 mg/kg of the hGH-XTEN and a plurality of subsequent doses of the hGH-XTEN in an amount of between about 0.05 mg/kg and about 3.0 mg/kg. In another embodiment, the doses are separated in time from each other by at least about 7 days. The package insert may further indicate different doses, dose ranges, and times between doses as described herein. In one additional embodiment, the human patient is an adult.

The following are examples of methods, treatment regimens, and compositions of the invention. It is understood that various other embodiments may be practiced, given the general description provided above.

### EXAMPLES

### Example 1

### Phase I Preliminary Results

A Phase 1 trial of safety, pharmacokinetics (PK) and pharmacodynamics (PD) of a single dose of a human growth hormone analogue (VRS-317) for subcutaneous administration in human adults with growth hormone deficiency has been completed and is detailed herein. VRS-317, a long acting rhGH fusion protein, the sequence of which is presented in FIG. 1, was evaluated in 50 adults with GHD in a 60-day, double-blind, randomized, placebo (PBO)-controlled, single ascending dose study of 0.05, 0.10, 0.20, 0.40 and 0.80 mg VRS-317/kg (ClinicalTrials.gov NCT01359488). VRS-317 is ˜5 times the mass of rhGH due to the addition of N- and C-terminal XTEN amino acids to extend the rhGH half-life. In monkeys, VRS-317 has complete bioavailability, rapid absorption, a half-life of ˜110 hr, and produces a sustained IGF-I response for one month after a single dose.

Initially, subjects were administered daily rhGH (min. of 28 days; dose range of 0.2-1.2 mg/day) until their serum IGF-I standard deviation (SD) score was stable in the range of −1.5 and +1.5. Subjects were then withdrawn from rhGH until the IGF-I SDS was <−1 and had fallen by ≧0.75 before treatment with VRS-317. The subjects were observed for 48 hrs after receiving VRS-317 or PBO. PK, PD (IGF-I) and paired fasting/post-prandial glucose were measured pre-dose and at various times over 30 days after a single SC dose of VRS-317 or PBO. Preliminary results from the trial were evaluated, including safety data for 28 subjects and PK/PD for 24 VRS-317 or PBO-treated subjects. PK/PD subjects (15M, 9F) had a mean (SD) age of 46 (12) yrs and BMI of 32 (7) kg/m2.

VRS-317 achieves a Tmax 2-3 days after a SC dose and has a long circulating half-life, potentially sufficient for monthly dosing. The mean maximal increases in IGF-I SDS were 0.33, 0.32, 0.96* and 1.32** in the PBO, 0.05, 0.10 and 0.20 mg/kg/month dosing groups, respectively (*p=0.012, **p=0.0005 (vs. PBO)). The percentages of subjects with IGF-I SDS above pre-VRS-317 levels for the initial two weeks were 16, 66 and 100% for the 0.05, 0.10 and 0.20 mg/kg/month groups, respectively. These single VRS-317 doses are equivalent to 0.31, 0.62, and 1.24 μg hGH/kg/day (typical AGHD dosing range for daily rhGH is 2-12 μg/kg/d).

There were no drug-related serious adverse events, withdrawals after dosing or unexpected, related adverse events or injection site lipoatrophy in the subjects. Mean fasting glucose, post-prandial glucose and change from fasting to postprandial showed no significant post-dosing changes in the subjects. No safety laboratory signals were observed in the subjects. In summary, in this trial of a single SC dose of VRS-317 in adults with GHD, graded responses of IGF-I generation were safely achieved at doses lower than those typically used with daily administration of rhGH over the course of one month.

### Example 2

### Completion of the Phase I Trial

Example 1 describes preliminary results of a Phase 1 Trial of Safety, Pharmacokinetics (PK) and Pharmacodynamics (PD) of a Single Dose of a New Human Growth Hormone Analogue (VRS-317) for Monthly Subcutaneous Administration in Adults with Growth Hormone Deficiency. The trial has concluded and the final results are reported herein.

VRS-317 was studied in 50 adults (10 placebo/40 active treated) with GHD in a 60-day, double-blinded randomized, placebo(PBO)-controlled, single ascending dose study of 0.05, 0.10, 0.20, 0.40 and 0.80 mg VRS-317/kg (ClinicalTrials.gov NCT01359488). The trial design is summarized as shown below.

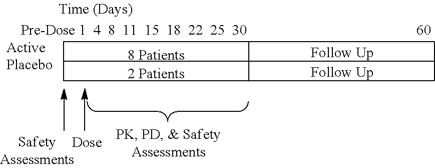

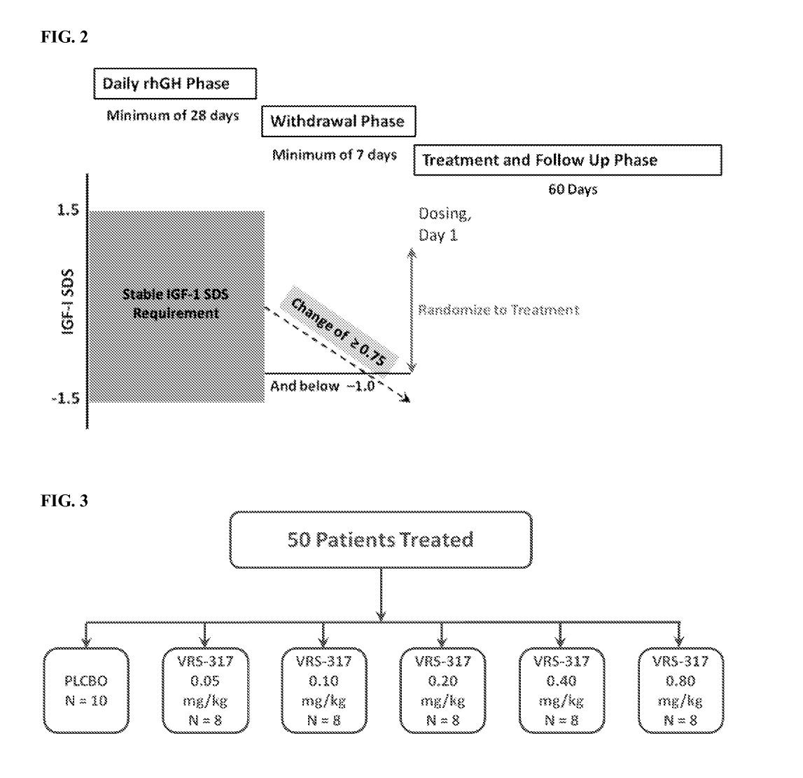

Patients were kept in the clinical unit for the first 48 hours after dosing. Immunogenicity (antibody samples) was evaluated at the following time points: pre-dose, 30 days, and 60 days following dosing. FIG. 2 depicts the study phases for the Phase 1 trial.

Objectives:

The objectives of the study included the following: to evaluate the safety and tolerability of a single subcutaneous (SC) dose in GHD patients; to determine single dose pharmacokinetics of VRS-317 administered SC; to evaluate evidence of VRS-317 bioactivity by changes from baseline in insulin-like growth factor-1 (IGF-I) and binding protein (IGFBP-3), and bone turnover (bone alkaline phosphatase); and to determine the dose to maintain a normal range (for appropriate age/gender) for IGF-I levels in adult patients for one month after administration of a single dose.

### Dosing:

Because of a demonstrated enhancement of the in vivo potency of GH in monkeys receiving VRS-317 (Cleland et al. 2012 supra), the VRS-317 dose range for the first dose in humans was selected to approximate the daily rhGH doses in the lower half of the typical dosing range for each 30 day interval (i.e., 0.03 to 0.5 mg rhGH/day or approximately 0.3 to 5.0 μg/kg/day). The selected VRS-317 doses were 0.05, 0.10, 0.20, 0.40 and 0.80 mg/kg administered as a single subcutaneous injection.

As shown in Table 2.1 below, VRS-317 single SC dose levels were at or below the equivalent mean adult GHD daily rhGH dose of 5 μg/kg/day.

TABLE 2.1

| | VRS-317 Dose | rhGH equivalent |
|---|---|---|
| Dose Level | (mg/kg - one dose) | (μg/kg/day × 30 days) |
| 1 | 0.05 | 0.31 |
| 2 | 0.10 | 0.62 |
| 3 | 0.20 | 1.24 |
| 4 | 0.40 | 2.48 |
| 5 | 0.80 | 4.97 |

Patient Disposition:

Enrolled subjects had growth hormone deficiency (GHD), as confirmed by a negative response to insulin (peak GH <5.0 ng/mL), arginine-GHRH (peak GH based on BMI) (Molitch M E, et al. 2011. J Clin Endocrinol Metab 96(6):1587-1609; Cook D M, et al. 2009. Endocrine practice: official journal of the American College of Endocrinology and the American Association of Clinical Endocrinologists 15 Suppl 2:1-29), glucagon (GH peak <3.0 ng/mL) (Yuen K C, et al. 2009. J Clin Endocrinol Metab 94(8):2702-2707), or at least 3 other pituitary hormone deficiencies and a low IGF-I for age and gender (Molitch M E, et al. 2011 supra). When GHD was due to a sellar region lesion, scans showed at least 6 months of stability. Treatments for other pituitary hormone deficiencies were stable for 2 months prior to study drug administration. Free T4 was in the normal range for all subjects when VRS-317 was administered. Each subject not taking daily glucocorticoid treatment had normal responses to a standard dose (250 μg) ACTH test to rule out secondary adrenal insufficiency. For female patients receiving estrogen, transdermal treatment was used and maintained throughout the study. IGF-I responses to daily rhGH were characterized in all subjects prior to study drug administration. Key exclusion criteria included the presence of significant concurrent disease (e.g. diabetes), active malignancy, anti-hGH antibodies at screening, pregnancy, lactation or the use of oral estrogens.

FIG. 3 summarizes the patient disposition in the study. No patients dropped out of the study after treatment with VRS-317 (or placebo).

Study Procedures and Method of Study:

Initially, all subjects were maintained on daily rhGH for a minimum of 28 days and until two successive IGF-I standard deviation scores (SDS), drawn at least one week apart, were within the range of −1.5 to 1.5 (+2.0 for males). Subjects were then withdrawn from daily rhGH until their IGF-I SDS decreased by at least 0.75 and had dropped to ≦−1.0. Subjects were then randomized to the treatment cohort enrolling at that time. On Day 1, all subjects received a single subcutaneous (SC) dose of VRS-317 or placebo administered with an insulin syringe with a 29 gauge needle. Pharmacokinetic and pharmacodynamic (PK/PD) samples were collected pre-dose and at 0.5, 1.0, 2, 4, 8, 12, 24, 36 and 48 hours after dosing. Additional PK/PD sampling was conducted on Days 4, 8, 11, 15, 18, 22, 25 and 30 after dosing. Glucose and lipid metabolism was assessed pre-dose and on Day 8, 15, 22, 30, 44 and 60 after dosing. Testing for anti-VRS-317 antibodies was conducted pre-dose and on Days 30 and 60 after dosing. Before proceeding to the next dosing level, safety data was reviewed. Laboratory safety assessments were performed prior to and at selected times after dosing. Tests included standard blood counts, biochemistries, postprandial glucose, hemoglobin A1c (HbA1c) and fasting levels of blood glucose, cholesterol, LDL, HDL and triglycerides.

Definition of Patient Populations:

The safety population consisted of all 50 randomized subjects. The PK/PD population consisted of 48 subjects receiving either VRS-317 or placebo and excluded two subjects who received inappropriate doses for their weight (one subject in the 0.80 mg/kg dose group and one subject in placebo group).

Assays:

VRS-317 concentrations in collected plasma were measured using an ELISA. The assay uses capture and detection antibodies to the XTEN and rhGH domains, respectively, to ensure detection of the intact molecule. Anti-VRS-317 antibodies were measured in samples taken pre-dose and at Day 30 and Day 60. Due to the potential for interference from high VRS-317 concentrations, samples were taken at the end of the dosing interval and assays were performed using solid-phase extraction with acid dissociation followed by a direct electrochemiluminescense assay. Anti-rhGH antibodies were measured in a direct ELISA. IGF-I was measured to bioanalytical standards using the acid extraction, IGF-II blocking radioimmunoassay (RIA), performed by Esoterix, Inc. (Calabasas Hills, Calif.). The lower limit of quantitation for the IGF-I assay is 15 ng/ml. IGFBP-3 was also measured by RIA at Esoterix. The lower limit of quantitation for the IGFBP-3 assay is 0.3 mg/L. Assay-specific standard deviation scores (SDS) for IGF-I and IGFBP-3 were developed using power transformed normative data (Esoterix, Calabasas Hills, Calif.) for the assays in use.

PK/PD Analysis:

VRS-317 PK parameters were estimated with non-compartmental techniques using WinNonLin™ professional v5.3 (Pharsight Corporation, Mountain View, Calif.). The IGF-I area under the curve after a single SC dose of VRS-317 was calculated using the linear trapezoid rule and average IGF-I was calculated by dividing IGF-I AUC by the time of the dosing interval.

Statistical Analysis:

Descriptive statistics and multivariate analyses were conducted according to a statistical analysis plan finalized prior to database lock. Laboratory parameters were analyzed for change from pre-dose baseline by ANCOVA with change as the dependent variable, treatment as cofactor and baseline value as covariate. P-values<0.05 defined statistical significance.

### Results

Patient Disposition and Characteristics:

Sixty-nine subjects were screened for enrollment; there were 19 screen failures and 50 subjects were randomized to five groups each consisting of 8 active- and 2 placebo-treated subjects. There were no withdrawals by subjects after randomization; all 50 randomized subjects completed the 60 day dose-evaluation period. There were 21 females and 29 males with a mean age of 44.7 years (Table 2.2). Age distributions were similar in each of the five dosing cohorts; however, some gender imbalance occurred between dosing arms (placebo and 0.10 mg/kg cohorts included 6 males, 2 females; 0.80 mg/kg cohort had 3 males, 5 females). Daily rhGH doses in the stability phase were in the range of 0.4 to 0.6 mg/day (4.1-5.8 μg/kg/day) on average across all the dose groups. For subjects randomized to VRS-317, the mean change in IGF-I SDS after rhGH withdrawal ranged from −1.7 to −2.4.

Table 2.2 below provides the characteristics of randomized subjects. Values are means (minimum, maximum) except as noted. Baseline is defined as the last measurement before study drug administration.

TABLE 2.2

| Treatment | 0.05 mg/kg | 0.10 mg/kg | 0.20 mg/kg | 0.40 mg/kg | 0.80 mg/kg | Placebo |
|---|---|---|---|---|---|---|
| Group | n = 8 | n = 8 | n = 8 | n = 8 | n = 8 | n = 10 |
| Age in years | 41.41 | 55.5 | 37.1 | 44.6 | 43.1 | 46.3 |
| (range) | (29, 57) | (48, 64) | (27, 59) | (29, 58) | (26, 59) | (26, 66) |
| Male, n (%) | 4 (50) | 6 (75) | 5 (62.5) | 5 (62.5) | 3 (37.5) | 6 (60) |
| BMI, kg/m² | 34.2 | 29.5 | 33.7 | 30.8 | 27.5 | 30.0 |
| (range) | (23, 45) | (20, 43) | (27, 44) | (23, 38) | (19, 34) | (23, 39) |
| Height, cm | 173.1 | 175.8 | 173.1 | 169.1 | 170.0 | 172.9 |
| (range) | (160, 180) | (160, 185) | (151, 183) | (153, 178) | (159, 188) | (159, 191) |
| Weight, kg | 103.2 | 90.9 | 101.3 | 88.1 | 80.7 | 90.9 |
| (range) | (58, 138) | (61, 124) | (70, 130) | (66, 115) | (49, 119) | (61, 144) |
| rhGH dose, | 5.1 | 4.1 | 5.0 | 5.2 | 5.8 | 5.2 |
| μg/kg/day | (1.5, 9.6) | (2.5, 7.3) | (1.9, 7.9) | (2.5, 10.5) | (2.5, 10.2) | (2.3, 10.3) |
| (range) | | | | | | |
| IGF-I SDS in | 0.11 | 0.17 | −0.21 | −0.63 | 0.02 | −0.12 |
| Daily rhGH | (−0.54, 1.4) | (−1.1, 1.5) | (−1.5, 0.9) | (−1.4, −0.1) | (−1.3, 1.6) | (−1.2, 0.9) |
| Phase (range) | | | | | | |
| IGF-I SDS in | −1.65 | −2.00 | −1.92 | −2.19 | −1.64 | −1.63 |
| Withdrawal | (−2.2, −1.3) | (−3.0, −1.4) | (−2.7, −1.1) | (−2.8, −1.0) | (−2.4, −1.0) | (−2.8, −1.2) |
| Phase (range) | | | | | | |
| Change in IGF-I | 1.76 | 2.17 | 1.71 | 1.56 | 1.62 | 1.51 |
| SDS (Daily to | | | | | | |
| Withdrawal | | | | | | |
| IGF-I SDS at | −1.74 | −2.27 | −2.09 | −2.30 | −1.75 | −1.52 |
| baseline (range) | (−2.3, −0.8) | (−2.9, −1.8) | (−3.0, −1.4) | (−2.9, −0.7) | (−2.9, −1.1) | (−3.1, −0.67) |

Pharmacokinetics:

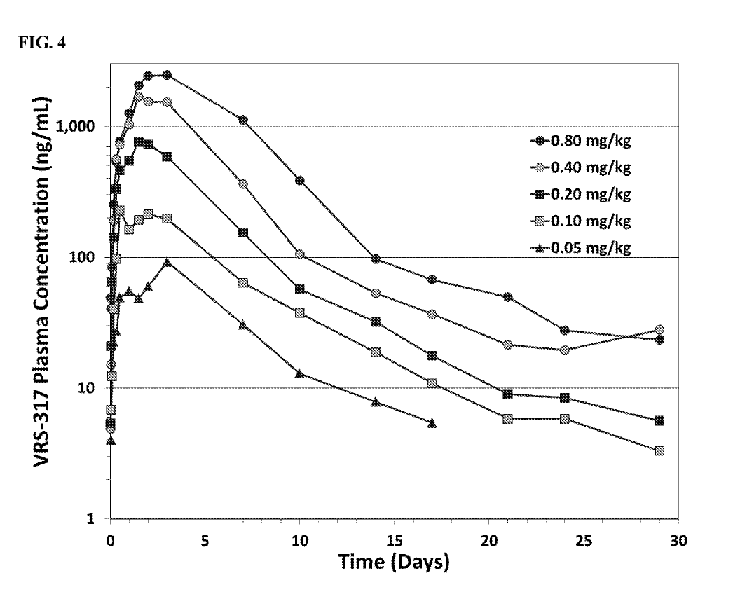

FIG. 4 shows the human pharmacokinetic (PK) profile for various single doses of VRS-317. FIG. 4 shows the time course of mean VRS-317 concentrations in adult GHD subjects receiving a single subcutaneous dose on Day 1. The variance bars are omitted for clarity; the mean coefficient of variation (SD/Mean) at Cmaxfor VRS-317 was 57% (all doses). Table 2.3 below provides the pharmacokinetic parameters (Mean±Standard Deviation) of VRS-317 in growth hormone-deficient adults following a single subcutaneous injection. A single SC dose resulted in rapid absorption and prolonged serum exposure to VRS-317 (FIG. 4). Mean maximal VRS-317 plasma concentrations (Cmax) were reached at 44 to 82 hours (Table 2.3). VRS-317 exposure was directly proportional to dose. There was a general trend for VRS-317 elimination half-life (t½) to increase with increasing dose. The mean t½was 131 hours at the highest dose tested (0.80 mg/kg) (Table 2.3). In multivariate analyses, the AUC₀₋ₜ for VRS-317 was highly correlated to dose (p<0.0001) but no significant age or gender effect was observed in this population.

TABLE 2.3
Pharmacokinetic Parameters Resulting from Administration of VRS-317

| Dose | Cₘₐₓ | Tₘₐₓ | AUC₀₋ₜ | AUC_{0-∞} | t_{1/2} |
|---|---|---|---|---|---|
| (mg/kg) | (ng/mL*) | (hr*) | (ng · hr/mL*) | (ng · hr/mL*) | (hr*) |
| 0.05 | 92 ± 29 | 46 ± 27 | 11,161 ± 3,395 | 11,706 ± 3,499 | 68 ± 18 |
| 0.10 | 354 ± 368 | 44 ± 21 | 33,365 ± 16,410 | 33,822 ± 16,343 | 85 ± 34 |
| 0.20 | 889 ± 606 | 50 ± 19 | 86,429 ± 67,201 | 87,291 ± 67,068 | 90 ± 50 |
| 0.40 | 1,968 ± 676 | 48 ± 17 | 241,280 ± 121,549 | 244,601 ± 125,167 | 109 ± 57 |
| 0.80 | 2,887 ± 1,345 | 82 ± 39 | 402,541 ± 124,653 | 407,421 ± 124,915 | 131 ± 62 |

*units ± S.D.
Cₘₐₓ = maximum concentration;
Tₘₐₓ = time to maximum concentration;
AUC₀₋ₜ = area under the curve from time zero to the last measurable time point;
AUC_{0-∞} = area under the curve from time zero to infinity;
t_{1/2} = terminal half-life.
The dose proportionality correlation coefficients (log:log) were 0.87 for Cmax and 0.93 for AUC₀₋ₜ.

No gender-based PK effect was observed. A significant (p=0.016) linear increase in t ½ was observed with increased dose. A dose proportional increase in Cmax and AUC was observed.

Pharmacodynamics:

IGF-I concentration was the primary pharmacodynamic marker employed for this study. The amplitude and duration of IGF-I exposure was directly proportional to VRS-317 dose (FIG. 5, Table 2.4). FIG. 5 illustrates a dose-response change in mean IGF-I SDS for 0.05, 0.10, 0.20, 0.40 and 0.80 mg VRS-317/kg. FIG. 5 shows the mean change in IGF-I SDS for placebo and 5 active dosing groups (note: one subject in the 0.80 mg/kg dose group was omitted from this figure because of an error in dose administration). The variance bars are omitted for clarity; the standard deviation at C maxfor IGF-I SDS for the five active dose groups ranged from 0.7 to 1.3.

TABLE 2.4

| | | IGF-I at | IGF-I at | IGF-I | IGF-I | IGF-I | IGF-I | Average |
|---|---|---|---|---|---|---|---|---|
| Dose | | Stability | Baseline | Cmax | Cmax | Tmax | AUC₀₋ₜ | IGF-I |
| (mg/kg) | N | (ng/mL*) | (ng/mL*) | (ng/mL*) | (SDS*) | (days*) | (ng · hr/mL*) | (ng/mL*) |
| Placebo | 9 | 188 ± 49 | 106 ± 47 | ND | ND | ND | ND | 102 ± 49 |
| 0.05 | 8 | 212 ± 41 | 97 ± 47 | 137 ± 58 | −1.1 ± 0.7 | 6.4 ± 6.5 | 2837 ± 1330 | 95 ± 44 |
| 0.10 | 8 | 170 ± 30 | 57 ± 18 | 105 ± 43 | −1.2 ± 0.9 | 5.0 ± 2.9 | 2214 ± 855 | 74 ± 29 |
| 0.20 | 8 | 214 ± 68 | 86 ± 30 | 196 ± 58 | −0.5 ± 0.9 | 4.1 ± 1.8 | 3541 ± 1260 | 118 ± 42 |
| 0.40 | 8 | 165 ± 44 | 70 ± 40 | 248 ± 87 | 0.9 ± 1.4 | 4.5 ± 1.4 | 3771 ± 1524 | 126 ± 51 |
| 0.80 | 7 | 197 ± 76 | 89 ± 31 | 280 ± 103 | 1.4 ± 1.3 | 5.7 ± 2.1 | 4884 ± 915 | 163 ± 31 |

*units ± S.D.
Stability refers to the time during daily rhGH treatment was given. Baseline refers to Day 1, prior to the dose of VRS_317 or placebo. Cₘₐₓ = maximum concentration; Tₘₐₓ = time to maximum concentration; AUC₀₋ₜ = area under the curve from time zero to the last measurable time point. The IGF-I AUC was calculated using the linear trapezoid rule. Average IGF-I was calculated by dividing AUC by the observation interval of 29 days. ND = not determined. The dose proportionality correlation coefficients (log:log) were 0.76 for baseline corrected Cmax and 0.76 for baseline corrected AUC₀₋ₜ.

FIG. 6 illustrates a sustained IGF-I response to a single dose of VRS-317 (Patients with baseline IGF-I SDS below −1.5). FIG. 6 shows the extent of normalization of IGF-I SDS after single SC dose administration of VRS-317 (note: data for 5 of the 39 subjects in FIG. 4 were excluded from FIGS. 5-6 because their baseline IGF-I SDS was ≧−1.5 and their inclusion would have exaggerated duration of normalization of IGF-I SDS).

An important observation was that the maxima for mean changes in IGF-I concentrations and IGF-I SDS appeared similar for the 0.40 mg/kg and 0.80 mg/kg groups. The similarity may have been caused by uneven distribution of subject characteristics affecting IGF-I responses to VRS-317. Therefore, an ANCOVA was used to examine the set of all post-dose values of IGF-I concentration for dependencies upon age, gender, treatment day, VRS-317 dose, treatment by day interaction (as factors) and baseline (pre-dose) IGF-I concentration (as covariate). Dose, day and dose and treatment by day interaction were all significant (p<0.0001) as were age (p=0.0034) and gender (p=0.0224). Higher doses, male gender and younger age were all associated with greater IGF-I responses.

The extent and duration to which IGF-I SDS were normalized were also VRS-317 dose-dependent. An analysis of subjects having an IGF-I SDS below −1.5 at the time of dosing indicated that VRS-317 increased the IGF-I SDS into the normal range of −1.5 to 1.5 in a dose-dependent manner (FIG. 6). IGF-I SDS was normalized for a mean of approximately 3 weeks for the 0.80 mg/kg group. This prolonged duration of normalization did not come at the expense of overexposure to IGF-I. The forty VRS-317 treated patients had a total of 513 post-dose IGF-I SDS determinations and only 8 values (1.6%) in 6 patients were above the normal range (SDS>+2). The individual IGF-I SDS values above +2 ranged from 2.01 to 3.59, occurred only in the 0.40 and 0.80 mg/kg groups, were usually were observed within 72 hours after dosing and had normalized by the subsequent sampling time.

IGFBP-3 SDS were low at baseline (Mean −1.28, SD 1.82) but increased with VRS-317 dosing. The time course of change in IGFBP-3 was similar to that of IGF-I. Maximal IGFBP-3 responses were generally observed at Day 4 or Day 8. The changes in IGFBP-3 were dose-dependent. At Day 8, the least square mean changes in IGFBP-3 were 0.05, 0.17, 0.55, 0.80, and 1.41 mg/L (IGFBP-3 SDS Cmax of −0.6 to 2.6) for the 0.05, 0.10, 0.20, 0.40 and 0.80 mg/kg dosing groups, respectively. In ANCOVA, IGFBP-3 responses were dependent on VRS-317 dose, day and baseline value (all p<0.0001) but no effects of age or gender were observed. At baseline the IGF-I/IGFBP-3 molar ratio was 0.22±0.05 and not statistically different between dose groups (p=0.49). Mean maximal molar ratio values were observed on Day 4 and increased with increasing VRS-317 dose (p<0.0001). The maximal mean molar ratio for the 0.80 mg/kg group was 0.47±0.11. The maximal molar ratio value for any subject was 0.65.

Safety Results:

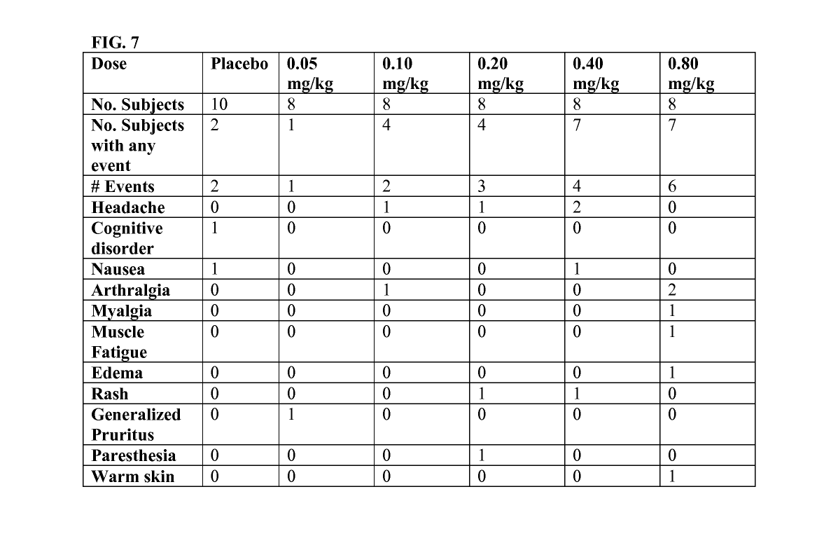

After review of safety data from a minimum of 8 patients exposed for a minimum of 7 days, patients were enrolled in all five planned dosing levels and there were no unexpected adverse events (AEs) related to the study drug. Non-laboratory AEs considered related to study drug by investigators were transient and mild (CTCAE Grade 1 except 2 cases of Grade 2) and occurred in a minority of subjects (FIG. 7).

FIG. 7 provides treatment-emergent adverse events possibly, probably or definitely related to study drug administration in the safety population (n=50) of GHD Adults. Injection site reactions and laboratory events are discussed herein. Many related events (headache (4), arthralgia (3), myalgia (1) and edema (1)) were of the type typically observed when rhGH is started in adult GHD patients. The 0.40 and 0.80 mg/kg dosing groups had the greatest number of any related AEs (7 in each group) but no specific event had a clear dose-relationship.

Injection site reactions were the most commonly reported drug-related adverse event.

Injection site erythema was noted in 30% of VRS-317 treated and 10% of placebo treated subjects. Injection site edema was noted in 10% of VRS-317 treated subjects and 10% of placebo treated subjects. Injection site pain or tenderness was observed in 15% of VRS-317 treated subjects. In general, for placebo and study drug-treated patients, injection site reactions appeared within 24 hours and were mild (Draize I, barely perceptible) and transient. There were no instances of injection site lipoatrophy or hypersensitivity reported through 60 days of post-treatment observation.

Glucose & Lipid Metabolism:

The safety of rhGH has been extensively characterized in animals and humans, and glucose intolerance has been observed and reported at certain doses of rhGH. Following administration of VRS-317, glucose and lipid metabolism was regularly assessed, including during the follow-up period. No significant changes were observed by day or dose (fasting glucose, post-prandial glucose, fasting insulin, and HbA1c). A clinically-significant reduction in cholesterol, triglycerides, and LDL was observed at the 0.8 mg/kg VRS-317 dose (data not shown). There were no reported safety events or clinically meaningful changes related to any glucose metabolism parameter. No patient had a glucose result in the diabetic range (fasting ≧126 mg/dL, post-prandial ≧200 mg/dL). All mean and individual values for HbA1c remained within the normal range. No clinically meaningful changes (≧0.2%) were noted in change from baseline HbA1c versus placebo in any treatment group. One patient each from the 0.10 and 0.20 mg/kg dosing group had worsening of previously elevated levels of serum cholesterol, LDL and triglycerides as possibly related AEs. However, at the highest VRS-317 dose (0.80 mg/kg), there was a temporal pattern of reduction in cholesterol, LDL and triglycerides, maximal at Day 8 and persisting through Day 22. The maximal percent decreases from baseline were 11.3 (p=0.0026), 14.6 (p=0.014) and 14.5% (p=0.19) for cholesterol, LDL and triglycerides, respectively. In summary, no observed data related to glucose and lipid metabolism resulted in safety concerns.

Antibody Assessments:

Non-specific binding was noted in the anti-hGH antibody assay. No subject had a significant titer (≧1:10) of specific anti-rhGH antibodies at screening and no subject tested positive at 7 days post-daily rhGH withdrawal. A single subcutaneous administration of VRS-317 to adult GHD patients previously treated with daily rhGH resulted in a minority of subjects (4 of 40) generating an anti-VRS-317 antibody response at low titer (3 of 4 subjects at 1:5, one subject at 1:25). Three of these 4 had non-specific binding in the anti-hGH antibody assay. Analysis of potential antibody effects on clinical or pharmacological endpoints was precluded by the low number of subjects testing positive for anti-VRS-317; there were no notable differences in IGF-I responses of these four subjects.

In summary, this study in adult GHD patients provides certain safety, pharmacokinetic, and pharmacodynamic (PD) information about VRS-317. Single doses of VRS-317 were found to be safe and well tolerated (see FIG. 7). Regarding the PK profile, AUC, Cmax, and half-life of VRS-317 was found to be proportional to dose. In addition, the duration of exposure to VRS-317 was found to increase with increased dose. Regarding the PD profile, the serum IGF-I normalized in a dose dependent manner and the duration of IGF-I normalization increases with increased dose. In addition, the dosing up to the midpoint of the daily rhGH dose range resulted in normalization of IG-1 for up to 3 weeks.

VRS-317 contains XTEN domains that increase the hydrodynamic radius and reduce binding affinity to the GH receptor (GHR), in vitro. Despite reduced binding affinity, durable pharmacodynamic responses are seen, in vivo, possibly relating to reduced rates of receptor mediated clearance of VRS-317 (Cleland et al. 2012 supra). The reduced rate of clearance prolongs serum residence times of VRS-317, resulting in enhanced ligand time on target. The terminal elimination half-life of VRS-317 at the highest dose was 131 hours; this represents a 30- to 60-fold increase over those reported in package inserts for daily rhGH products.

The current study was the first in humans for VRS-317 and extends prior knowledge about long-acting rhGH because it represents the most prolonged duration of action of any rhGH analogue in the treatment of adults with GHD. All subjects were adults with GHD diagnosed in accordance with current consensus guidelines of The Endocrine Society, the American Association of Clinical Endocrinologists and the Growth Hormone Research Society. There was a slight preponderance of male subjects (29M, 21F) but the numbers of each gender were adequate to test for gender effects on drug distribution and pharmacodynamic effects. Each subject was initially stabilized on daily rhGH injections and, to achieve stable IGF-I SD scores within the normal range, had been taking 0.2 to 1.0 mg hGH/day (mean 0.6 mg/day) or 1.5 to 10.5 μg/kg/day. Following discontinuation of daily rhGH, IGF-I SDS decreased in all subjects with group mean decrements of 1.7 to 2.5 SD. Subjects requiring daily medication that could alter sensitivity to rhGH (e.g., insulin, oral estrogens, anti-inflammatory doses of glucocorticoids) were excluded from this first dosing study of VRS-317.

Over the VRS-317 dosing range, drug exposure parameters (Cmax and AUC) were directly and highly proportional to dose. In general, both the amplitude and duration of exposure increases with increased VRS-317 dose. No gender or age effects were detected in the VRS-317 dose-exposure relationship. VRS-317 was safe and well-tolerated at all dose levels suggesting that greater dose exposures can be explored in future human studies. The pharmacodynamic (IGF-I and IGFBP-3) responses to VRS-317 were also directly proportional to dose, with amplitude and duration increasing with increased dose. At the highest dose, the mean IGF-I SDS was maintained above −1.5 for approximately 3 weeks. Given the demonstrated proportionality between dose and duration, the duration of IGF-I normalization could be extended by increased VRS-317 doses. Over the dose range assessed in this study, the results support that the duration of IGF-I normalization does not come at the expense of over-exposure to IGF-I: only 1.6% of observed IGF-I SDS were ≧2 and these elevations were transient. There were age and gender effects on IGF-I responses to VRS-317 such that females and older subjects had lower responsiveness than males. Based on these analyses, females and older subjects are anticipated to have lower IGF-I responses to VRS-317. Gender differences for IGF-I induction are well known for daily rhGH and are likely due to estrogen effects on IGF-I producing cells. Similar to the effects of daily rhGH, IGF-I induction by VRS-317 in adults may be lower in females than in males.

VRS-317 was administered at doses ranging from 0.05 to 0.80 mg/kg; approximating daily rhGH doses of 0.3 to 5 μg/kg/d over 30 days. Over this range, a single dose of VRS-317 was safe and well-tolerated. There were no treatment emergent serious adverse events or suspected unexpected serious adverse reactions. No subject withdrew from the study after dosing; all subjects completed the protocol-specified 60 day safety observation period. Minimal, transient erythema at the injection site(s) was the most commonly reported adverse event. Other events considered as possibly, probably or definitely related to study drug were typical of those seen when adult GHD patients receive replacement therapy. These events were transient and were categorized as mild-moderate. No injection site lipoatrophy was observed. Surveillance for VRS-317 alterations in carbohydrate metabolism included serial measurements of fasting glucose and insulin, post-prandial glucose and HbA1c. No clinically-meaningful temporal or dose-related changes were observed in any of these parameters, indicating that the prolonged action and delayed clearance of VRS-317 did not confer any additional risk to overall glycemic safety in these patients. These findings are in accordance with previous studies with low dose daily rhGH (Yuen K C et al. 2009, supra; Spina LDC, et al. 2004. Growth Hormone & IGF Research 14(1):45-51; Hana V, et al. 2004, Clinical Endocrinology 60(4):442-450; Bulow B et al. 2004. Clinical Endocrinology 61(6):683-691; Yuen K C et al. 2007, Diabetes, Obesity & Metabolism 9(1):11-22) but in contrast to other studies showing elevated glucose and insulin with decreased insulin sensitivity indices during long-term daily rhGH treatment (Boguszewski C L et al. 2005 European Journal of Endocrinology 152(1):67-75; Moller N et al. 2009. Endocrine Reviews 30(2):152-177; Christopher M et al. 1998. J Clin Endocrinol Metab 83(5):1668-1681). Although two subjects in a lower dose group had increases in previously elevated levels of LDL, total cholesterol and triglycerides, there was a temporal pattern of decrease in these parameters at the highest VRS-317 dose level (0.80 mg/kg). It is considered as likely that rhGH dose and duration effects as well as individual susceptibility will influence glucose, lipid and insulin responses. Continued surveillance for alterations in lipid and glucose parameters is warranted during subsequent chronic dosing trials.

Four of the 40 VRS-317 treated subjects had detectable anti-VRS-317 antibodies appearing at Day 30 and/or 60 after VRS-317 dosing. These subjects had received VRS-317 doses of 0.2 mg/kg (1 subject), 0.40 mg/kg (2 subjects) or 0.80 mg/kg (1 subject). Three of these four had had non-specific binding in the anti-rhGH antibody screening assay.

In conclusion, single dose administration of VRS-317 is safe and well tolerated over the range of doses studied and provides prolonged normalization of IGF-I responses in adults with GHD. The safety and PK/PD profiles suggest VRS-317 doses may be further increased to prolong IGF-I responses in this population. Given its delayed clearance, VRS-317 has the potential for monthly dosing in adults with GHD.

What is claimed is:

1. A method of treating human growth hormone deficiency (GHD), comprising administering to a human patient with GHD a human growth hormone-XTEN (hGH-XTEN) fusion protein comprising an amino acid sequence having at least about 90% sequence identity to SEQ ID NO:1, as a therapeutically effective bodyweight adjusted bolus dose between about 0.05 mg/kg and about 3.0 mg/kg.

2. The method of claim 1, wherein the bolus dose is administered every week, every two weeks, every three weeks, or monthly.

3. The method of claim 2, wherein the administration of the bolus dose is monthly.

4. The method of any one of claims 1 to 3, wherein the bolus dose of hGH-XTEN fusion protein is between about 0.05 mg/kg and about 0.8 mg/kg or between about 0.8 mg/kg and about 1.2 mg/kg.

5. The method of any one of claims 1 to 3, wherein the bolus dose is administered subcutaneously.

6. The method of any one of claims 1 to 3, wherein the human patient has a serum IGF-I standard deviation score (SDS) between about −2.0 and about 2.0 following administration.

7. The method of claim 6, wherein the IGF-I SDS is selected from the group consisting of greater than about −2.0, greater than about −1.5, greater than about −1.0, greater than about −0.5, greater than about 0, greater than about 0.5, greater than about 1.0, and greater than about 1.5.

8. The method of claim 6, wherein the human patient exhibits said serum IGF-I SDS following administration of the bolus dose, wherein the administration is weekly, every two weeks, every three weeks, or monthly.

9. The method of any one of claims 1 to 3, wherein administration of the bolus dose results in a normalization of IGF-I SDS in the human patient for at least about 7 days, at least about 10 days, at least about 14 days, at least about 16 days, or at least about 21 days.

10. The method of any one of claims 1 to 3, wherein the bolus dose is selected from the group consisting of about 0.05 mg/kg, about 0.1 mg/kg, about 0.2 mg/kg, about 0.4 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, and about 3.0 mg/kg.

11. The method of any one of claims 1 to 3, wherein the hGH-XTEN fusion protein comprises the amino acid sequence of SEQ ID NO:1.

12. A method of treating human growth hormone deficiency (GHD), comprising administering to a human patient with GHD a human growth hormone-XTEN (hGH-XTEN) fusion protein comprising an amino acid sequence having at least about 90% sequence identity to SEQ ID NO:1, as a therapeutically effective bodyweight adjusted bolus dose of the hGH-XTEN fusion protein equivalent to less than an hGH/kg/day dosage between about 2 μg hGH/kg/day and about 20 μg hGH/kg/day.

13. The method of claim 12, wherein the bolus dose is administered every week, every two weeks, every three weeks, or monthly.

14. The method of claim 12, wherein the administration of the bolus dose is monthly.

15. The method of any one of claims 12 to 14, wherein the hGH/kg/day dosage is over about 30 days.

16. The method of any one of claims 12 to 14, wherein the bolus dose is administered subcutaneously.

17. The method of any one of claims 12 to 14, wherein the human patient has a serum IGF-I standard deviation score (SDS) between about −2.0 and about 2.0 following administration.

18. The method of claim 17, wherein the IGF-I SDS is selected from the group consisting of greater than about −2.0, greater than about −1.5, greater than about −1.0, greater than about −0.5, greater than about 0, greater than about 0.5, greater than about 1.0, and greater than about 1.5.

19. The method of claim 17, wherein the human patient exhibits said serum IGF-I SDS following administration of the bolus dose, wherein the administration is weekly, every two weeks, every three weeks, or monthly.

20. The method of any one of claims 12 to 14, wherein the bolus dose is equivalent to less than an hGH/kg/day dosage selected from the group consisting of about 2 μg hGH/kg/day, about 4 μg hGH/kg/day, about 6 μg hGH/kg/day, about 8 μg hGH/kg/day, about 10 μg hGH/kg/day, about 12 μg hGH/kg/day, about 14 μg hGH/kg/day, about 16 μg hGH/kg/day, about 18 μg hGH/kg/day, about 18.6 μg hGH/kg/day, and about 20 μg hGH/kg/day.

21. The method of any one of claims 12 to 14, wherein the hGH-XTEN fusion protein comprises the amino acid sequence of SEQ ID NO:1.

22. A method of treating human growth hormone deficiency (GHD) in a human patient, comprising administering to the patient with GHD a human growth hormone-XTEN (hGH-XTEN) fusion protein comprising an amino acid sequence having at least about 90% sequence identity to SEQ ID NO:1, as a therapeutically effective bodyweight adjusted bolus dose that is effective to maintain the patient's serum IGF-I standard deviation score (SDS) between about −2.0 and about 2.0 for at least 7 days after administration of the bolus dose.

23. The method of claim 22, wherein the bolus dose is between about 0.05 mg/kg and about 0.8 mg/kg, between about 0.8 mg/kg and about 1.2 mg/kg, or between about 0.05 mg/kg and about 3.0 mg/kg.

24. The method of claim 22 or 23, wherein said bolus dose is effective to maintain the patient's serum IGF-I SDS between about −2.0 and about 2.0 for at least 20 days after administration of the bolus dose.

25. A method of treating human growth hormone deficiency (GHD) in a human patient, comprising administering to the patient with GHD a human growth hormone-XTEN (hGH-XTEN) fusion protein comprising an amino acid sequence having at least about 90% sequence identity to SEQ ID NO:1, as a therapeutically effective bodyweight adjusted bolus dose that is effective to maintain a plasma concentration of said fusion protein in the patient at more than about 10 ng/mL for a period of at least 10 days after administration of the bolus dose.

26. The method of claim 25, wherein the bolus dose is between about 0.05 mg/kg and about 0.8 mg/kg, between about 0.8 mg/kg and about 1.2 mg/kg, or between about 0.05 mg/kg and about 3.0 mg/kg.

27. The method of claim 25 or 26, wherein said bolus dose is effective to maintain a plasma concentration of said fusion protein in the patient at more than about 10 ng/mL for a period of at least about 14 days, at least 20 days, at least about 28 days, or at least about 30 days after administration of the bolus dose.

28. The method of claim 25 or 26, wherein said bolus dose is effective to maintain a plasma concentration of said fusion protein in the patient at more than about 10 ng/mL for a period of at least 20 days or at least about 30 days after administration of the bolus dose.

29. The method of claim 25 or 26, wherein said bolus dose is effective to maintain a plasma concentration of said fusion protein in the patient at more than about 100 ng/mL for a period of at least 10 days after administration of the bolus dose.

30. A method of treating human growth hormone deficiency (GHD) in a human patient, comprising administering to the patient with GHD a human growth hormone-XTEN (hGH-XTEN) fusion protein comprising an amino acid sequence having at least about 90% sequence identity to SEQ ID NO:1, as a therapeutically effective bodyweight adjusted bolus dose that is effective in increasing the patient's IGF-I SDS by at least 0.5 or at least 1.0 above the subject's baseline IGF-I SDS in the absence of a clinically significant level of side-effects selected from the group consisting of headache, arthralgia, myalgia, edema, nausea, and muscle fatigue after administration of the bolus dose.

31. The method of any one of claims 22, 25, and 30, wherein said bolus dose is administered subcutaneously.

32. The method of any one of claims 22, 25, and 30, wherein the hGH-XTEN fusion protein comprises the amino acid sequence of SEQ ID NO:1.

33. The method of any one of claims 22, 25, and 30, wherein the human patient has a clinically significant reduction in at least one parameter selected from serum cholesterol, serum triglycerides, and serum low-density lipoprotein (LDL) after administration of the bolus dose, wherein the administration is selected from the group consisting of weekly, every two weeks, every three weeks, and monthly.

34. A bolus dose of an hGH-XTEN fusion protein comprising an amino acid sequence having at least about 90% sequence identity to SEQ ID NO:1, wherein the bolus dose is a therapeutically effective bodyweight adjusted bolus dose comprising between about 0.05 mg/kg and about 3.0 mg/kg of hGH-XTEN fusion protein.

35. The bolus dose of claim 34 for use in treating human growth hormone deficiency (GHD) in a subject in need.

36. The bolus dose of claim 34 or 35, wherein the hGH-XTEN fusion protein comprises the amino acid sequence of SEQ ID NO:1.

37. The bolus dose of claim 34 or 35, which is formulated for subcutaneous administration.

38. An hGH-XTEN fusion protein comprising an amino acid sequence having at least about 90% sequence identity to SEQ ID NO:1 for use in a method for the treatment of human growth hormone deficiency (GHD) in a human patient, wherein the method comprises administering a therapeutically effective bodyweight adjusted bolus dose of the hGH-XTEN fusion protein at a dose between about 0.05 mg/kg and about 3.0 mg/kg.

39. Use of an hGH-XTEN fusion protein comprising an amino acid sequence having at least about 90% sequence identity to SEQ ID NO:1 in the manufacture of a medicament for the treatment of GHD, wherein the hGH-XTEN fusion protein is administered to a human patient as a therapeutically effective bodyweight adjusted bolus dose of the hGH-XTEN fusion protein at a dose between about 0.05 mg/kg and about 3.0 mg/kg

40. The hGH-XTEN fusion protein of claim 38 or the use of claim 39, wherein the bolus dose is administered every week, every two weeks, every three weeks, or monthly.

41. The hGH-XTEN fusion protein of claim 38 or the use of claim 39, wherein the hGH-XTEN fusion protein comprises the amino acid sequence of SEQ ID NO:1.

42. The hGH-XTEN fusion protein of claim 38 or the use of claim 39, wherein the bolus dose is administered subcutaneously.

43. The hGH-XTEN fusion protein of claim 38 or the use of claim 39, wherein the human patient has a serum IGF-I standard deviation score (SDS) between about −2.0 and about 2.0 following administration of the bolus dose.

44. The hGH-XTEN fusion protein or use of claim 43, wherein the IGF-I SDS is selected from the group consisting of greater than about −2.0, greater than about −1.5, greater than about −1.0, greater than about −0.5, greater than about 0, greater than about 0.5, greater than about 1.0, and greater than about 1.5.

45. The hGH-XTEN fusion protein or use of claim 43, wherein the administration is weekly, every two weeks, every three weeks, or monthly.

46. The hGH-XTEN fusion protein of claim 38 or the use of claim 39, wherein the human patient has a clinically significant reduction in at least one parameter selected from serum cholesterol, serum triglycerides, and serum LDL after administration of the bolus dose, wherein the administration is weekly, every two weeks, every three weeks, or monthly.

47. A method of increasing the efficacy of human growth hormone (hGH) therapy in a human patient, comprising
(a) monitoring the IGF-I standard deviation score (SDS) in a plasma or serum sample obtained from the patient during an initial dosage period of administration of an initial dose of human growth hormone-XTEN (hGH-XTEN) fusion protein comprising an amino acid sequence having at least about 90% sequence identity to SEQ ID NO:1; and
(b) determining a subsequent dose of hGH-XTEN fusion protein administered over a subsequent dosage period based on the IGF-I SDS observed during the initial dosage period, wherein the subsequent dose improves the efficacy of the treatment during the subsequent dosage period.

48. A kit comprising
(i) a container which holds a pharmaceutical composition comprising a human growth hormone-XTEN (hGH-XTEN) fusion protein comprising an amino acid sequence having at least about 90% sequence identity to SEQ ID NO:1, and
(ii) a package insert associated with said container, wherein the package insert indicates that said composition is for the treatment of growth hormone deficiency by administration of an initial dose of the hGH-XTEN fusion protein between about 0.05 mg/kg and about 3.0 mg/kg and a plurality of subsequent doses of the hGH-XTEN fusion protein between about 0.05 mg/kg and about 3.0 mg/kg, wherein the doses are administered every week, every two weeks, every three weeks, or monthly.
